(19) United States
(12) Patent Application Publication
(10) Pub. No.: US 2016/0192689 A1
(43) Pub. Date: Jul. 7, 2016

(54) ENCAPSULATED FUNCTIONAL FOOD COMPOSITIONS

(71) Applicant: WIKIFOODS, INC., Cambridge, MA (US)

(72) Inventor: Gregory T. Horn, Lighthouse Point, FL (US)

(21) Appl. No.: 14/908,789

(22) PCT Filed: Jul. 31, 2014
(86) PCT No.: PCT/US14/49066

§ 371 Date: Jan. 29, 2016

Related U.S. Application Data
(60) Provisional application No. 61/860,586, filed on Jul. 31, 2013.
(60) Provisional application No. 61/860,609, filed on Jul. 31, 2013.
(60) Provisional application No. 61/860,652, filed on Jul. 31, 2013.
(60) Provisional application No. 61/860,629, filed on Jul. 31, 2013.
(60) Provisional application No. 61/860,625, filed on Jul. 31, 2013.
(60) Provisional application No. 61/860,591, filed on Jul. 31, 2013.
(60) Provisional application No. 61/860,623, filed on Jul. 31, 2013.
(60) Provisional application No. 61/860,595, filed on Jul. 31, 2013.
(60) Provisional application No. 61/860,617, filed on Jul. 31, 2013.
(60) Provisional application No. 61/860,599, filed on Jul. 31, 2013.
(60) Provisional application No. 61/947,803, filed on Mar. 4, 2014.

(51) Int. Cl. A23L 1/29; A61K 35/745; A61K 35/747; A61K 31/702; A61K 9/00; A61K 35/742; A61K 31/733

(52) U.S. Cl. CPC A23L 1/3014; A23L 1/0029; A23L 1/308; A23L 1/30; A61K 35/742; A61K 35/745; A61K 35/747; A61K 31/733; A61K 31/702; A61K 9/0056; A23V 2002/00

(57) ABSTRACT

A functional food composition comprising an edible or potable substance, a cross-linked edible matrix encapsulating the edible or potable substance, a probiotic, and a prebiotic, with the proviso that the edible or potable substance and the matrix do not both contain the probiotic or both contain the prebiotic. The encapsulated composition provides certain advantages in its transport, handling and consumption. The edible membrane matrix can be constructed to include edible particles that impart advantages to the performance of the membrane matrix, provide functional nutrition, health and well-being benefits, therapeutic treatment to the consumer, and/or enhance consumer gustatory experience. In particular aspects, the functional food composition provides nutritive and health benefits for disease treatment and prevention, and health maintenance through the incorporation of certain biologically active compounds in the edible substance and/or as particles in the membrane matrix.

### RELATED APPLICATIONS

This application claims the benefit of U.S. Patent Application No. 61/860,586, filed on Jul. 31, 2013, U.S. Patent Application No. 61/860,629, filed on Jul. 31, 2013, U.S. Patent Application No. 61/860,609, filed on Jul. 31, 2013, U.S. Patent Application No. 61/860,652, filed on Jul. 31, 2013, U.S. Patent Application No. 61/860,625, filed on Jul. 31, 2013, U.S. Patent Application No. 61/860,591, filed on Jul. 31, 2013, U.S. Patent Application No. 61/860,623, filed on Jul. 31, 2013, U.S. Patent Application No. 61/860,595, filed on Jul. 31, 2013, U.S. Patent Application No. 61/860,617, filed on Jul. 31, 2013, U.S. Patent Application No. 61/860,599, filed on Jul. 31, 2013 and U.S. Patent Application No. 61/947,803, filed on Mar. 4, 2014; the entire teachings of these applications are incorporated herein by reference.

### TECHNICAL FIELD

This disclosure relates to functional food compositions, membrane encased edible compositions, and more particularly to fully encased, functional food compositions.

### BACKGROUND

Functional foods provide nutritional components that are important for health maintenance. These food compositions contain compounds that are biologically active or bioavailable, such as probiotics, amino acids, multivitamins, and antioxidants, and often are found to be useful for the treatment of disease and disorders or the maintenance of normal health states. However, delivery systems for bioavailable compounds often can have insufficient viability, resulting in suboptimal bioactive quantities and bioavailability of these compounds. Many functional compounds are beneficial to hosts only when consumed in sufficient quantities. Therefore, consumers continually seek functional food compositions with enhanced effectiveness.

### SUMMARY

This disclosure relates to a variety of functional food compositions. Functional food compositions are designed to deliver sufficient quantities and quality of bioactive and bioavailable compositions using edible or potable substances encapsulated in an edible membrane matrix. The encapsulated composition provides certain advantages in its transport, handling and consumption. The edible membrane matrix can be constructed to include edible particles that impart advantages to the performance of the membrane matrix, provide functional nutrition, health and well-being benefits, therapeutic treatment to the consumer, and/or enhance consumer gustatory experience. In particular aspects, the functional food composition provides nutritive and health benefits for disease treatment and prevention, and health maintenance through the incorporation of certain biologically active compounds in the edible substance and/or as particles in the membrane matrix.

Provided herein is a functional food composition and method for making, comprising an edible or potable substance, a cross-linked matrix encapsulating the edible or potable substance, a probiotic, and a prebiotic, with the proviso that the edible or potable substance and the matrix do not both contain the probiotic or both contain the prebiotic. The matrix can comprise the prebiotic and the edible or potable substance component comprises the probiotic, or the matrix comprises the probiotic and the edible or potable substance comprises the prebiotic. The functional food composition can comprise a probiotic concentration from about 1.0 to about 100 billion colony forming units in an individual edible transport vessel, and the probiotic can be an organism selected from the genera group consisting of Aspergillus, Bacillus, Bacteroides, Bifidobacterium, Brettanomyces, Enterococcum, Kluyveromyces, Lactobacillus, Lactococcus, Leuconostoc, Pediococcus, Propionibacterium, Saccharomyces, Shewanella, Streptococcus, Torulaspora, Vagococcus, and any derivatives, strains, and combinations thereof.

The prebiotic of the functional food composition can be one of the group consisting of galacto-oligosaccharides, inulin, oligofructose, isomalto-oligosaccharides, lactulose, lactosucrose, transgalacto-oligosaccharides, soybean oligosaccharides, tagatose, xylo-oligosaccharides, and combinations thereof. The prebiotic concentration can be from about 1.0 to about 30.0 grams in an individual edible transport vessel.

The functional food composition can comprise additional edible particles in the cross-linked matrix. The prebiotic, the probiotic, and/or additional edible particles can provide enhanced performance to the matrix.

The prebiotic, the probiotic, and/or additional edible particles can comprise a size having a volume mean distribution between about 0.1 microns and about 1.0 microns, between about 0.1 microns and about 10.0 microns, between about 0.1 microns and about 100.0 microns, between about 0.1 microns and about 1.0 millimeters, or between about 0.1 and about 3 millimeters.

The functional food composition can be used for the normalization of gastro-intestinal flora in a subject, for the prevention and treatment of a gastrointestinal disorder, for the prevention and treatment of a systemic disorder, to improve immune function, for improved digestion, and to decrease intestinal gas production.

Provided is a method for maintaining or improving digestive health by administering to an individual in need thereof a functional food composition as described herein. The functional food composition can be administered in an amount sufficient to increase the gastrointestinal microflora concentration of an individual. The functional food composition can be administered in an amount sufficient to increase the fecal microflora concentration of an individual. The functional food composition can be administered in an amount sufficient to maintain normal gastrointestinal microflora concentration of an individual. The functional food composition can be administered in an amount sufficient to provide relief from gastrointestinal bloating of an individual.

Provided is a method for the prevention and treatment of a gastrointestinal disorder by administering to an individual in need thereof a functional food composition as described herein, where the gastrointestinal disorder comprises at least one of the group comprising diarrhea, Traveler's Diarrhea, antibiotic-associated diarrhea, inflammatory bowel disease, excessive stomach acid, dyspepsia, constipation, irritable bowel syndrome, Crohn's Disease, lactose intolerance, and pathogenic infection.

The functional food can be administered in an amount sufficient to prevent or treat the establishment of opportunistic gastrointestinal pathogens.

Provided is a method for the treatment of a systemic disorder by administering to an individual in need thereof a functional food composition as described herein, where the systemic disorder comprises at least one of the group consisting of elevated cholesterol, hypertension, acid reflux disease, colon cancer, and obesity.

Provided is a method for improving immune response by administering to an individual in need thereof a functional food composition as described herein, where improving immune response further comprises lowering the severity of at least one of the group consisting of diarrhea, Traveler's Diarrhea, antibiotic-associated diarrhea, inflammatory bowel disease, excessive stomach acid, dyspepsia, constipation, irritable bowel syndrome, Crohn's Disease, lactose intolerance, and pathogenic infection.

This disclosure also relates to the delivery of bioflavonoids, carotenes and elastic tissue components using edible or potable substances encapsulated in an edible membrane matrix. In particular aspects, the functional food provides functionally nutritive benefits for integument disorder prevention and health, beauty and wellness maintenance through the incorporation of certain biologically active compounds in the edible substance and/or as particles in the membrane matrix.

Provided herein is a functional food composition and method for making, comprising an edible or potable substance, a cross-linked matrix encapsulating the edible or potable substance, the cross-linked matrix comprising an edible polymer, and an integument health compound comprising a bioflavonoid, a carotene, and an elastic tissue component.

The functional food composition can include a bioflavonoid derived from at least one of the group consisting of Camellia sinensis, Theobroma cacao, varieties thereof, strains thereof, cultivars thereof, and combinations thereof.

The functional food composition can include bioflavonoid selected from the group consisting of esveratrol, quercetin, rutin, catechin, epicatechin, proanthocyanidin, cocoaflavanol, and combinations thereof.

The functional food composition can include an essential fatty acid selected from the group consisting of α-linolenic acid, eicosapentanoic acid, docosahexaenoic acid, and combinations thereof.

The functional food composition can include a carotene selected from the group consisting of α-carotene, β-carotene, γ-carotene, δ-carotene, ε-carotene, lycopene, and combinations thereof.

The functional food composition can include a connective tissue component selected from the group consisting of collagen, elastin, and combinations thereof.

The functional food composition can comprise additional edible particles in the cross-linked matrix. The cardiovascular health compound and/or additional edible particles can provide enhanced performance to the matrix.

The functional food composition of the integument health compound and/or additional edible particles can have a size with a volume mean distribution between about 0.1 microns and about 1.0 microns, between about 0.1 microns and about 10.0 microns, between about 0.1 microns and about 100.0 microns, between about 0.1 microns and about 1.0 millimeters, or between about 0.1 and about 3 millimeters.

Provided herein is a method of preparing a functional food composition, comprising the steps of providing an edible or potable substance, an edible unpolymerized matrix, and an integument health compound comprising a bioflavonoid, a carotene, and an elastic tissue component, combining the edible or potable substance and/or the edible unpolymerized matrix with the integument health compound, and encapsulating the edible substance with the edible matrix. The method can further comprise the step of polymerizing the edible matrix.

The functional food composition can be used for maintenance of skin, for the maintenance of nails, for the maintenance of hair, for the prevention and treatment of dry skin, for the prevention and treatment of skin wrinkles, for the prevention and treatment of skin spots, for the prevention and treatment of brittle nails, for the prevention and treatment of hair loss.

Provided is a method for the maintenance of skin health by administering to an individual in need thereof a functional food composition as described herein.

Provided is a method for the maintenance of hair health by administering to an individual in need thereof a functional food composition as described herein.

Provided is a method for the maintenance of nail health by administering to an individual in need thereof a functional food composition as described herein.

Provided is a method for the prevention and treatment of hair loss by administering to an individual in need thereof a functional food composition as described herein.

Provided is a method for the prevention and treatment of brittle nails by administering to an individual in need thereof a functional food composition as described herein.

Provided is a method for the prevention and treatment of skin wrinkles by administering to an individual in need thereof a functional food composition as described herein.

Provided is a method for the prevention and treatment of skin spots by administering to an individual in need thereof a functional food composition as described herein.

This disclosure also relates to the delivery of compounds useful for weight loss using edible or potable substances encapsulated in an edible membrane matrix. In particular aspects, the functional food provides functionally nutritive benefits for disease prevention and health wellness through the incorporation of certain biologically active compounds in the edible substance and/or as particles in the membrane matrix.

Provided herein is a functional food composition and method for making, comprising an edible or potable substance, a cross-linked matrix encapsulating the edible or potable substance, and particles for weight loss. The functional food composition can include a weight loss compound selected from the group consisting of an appetite suppressant, fat uptake inhibitors, gastrointestinal fillers, and thermogenic compounds.

The functional food composition can include a weight loss compound selected from the group consisting of thylakoids, thylakoid extracts, and combinations thereof.

The functional food composition can include at least one of the group consisting of a vitamin, a mineral, an amino acid, an antioxidant, an anti-inflammatory agent, and an essential fatty acid.

The functional food composition can include at least one of the group consisting proteinase inhibitor II, green coffee bean extract, chlorogenic acid, green tea leaf extract, polyphenols, ashwagandha extract, xanthum gum, and pinolenic acid, hoodia, chitosan, chromium picolinate, conjugated linoleic acid, glucomannan, green tea extract, guar gum, guarana, guggal, senna, ephedra, bitter orange, fucoxanthin, white bean extract, vitamin D, human chorionic gonadotropin, resveratrol, capsaicin, chia, hoodia, L-carnitine, raspberry ketones, banana leaf, red clover, ginger, almonds, acai berry, flax seeds, leucine, and lipodrene.

The functional food composition can comprise additional edible particles in the cross-linked matrix. The edible particles weight loss and/or additional edible particles can provide enhanced performance to the matrix.

The functional food composition can comprise edible particles for weight loss and/or additional edible particles can have a size with a volume mean distribution between about 0.1 microns and about 1.0 microns, between about 0.1 microns and about 10.0 microns, between about 0.1 microns and about 100.0 microns, between about 0.1 microns and about 1.0 millimeters, or between about 0.1 and about 3 millimeters.

Provided herein is a method of preparing a functional food composition, comprising the steps of providing an edible or potable substance, an edible unpolymerized matrix, and edible particles for weight loss, combining the edible or potable substance and/or the edible unpolymerized matrix with the edible particles for weight loss, and encapsulating the edible substance with the edible matrix. The method can further comprise the step of polymerizing the edible matrix.

The functional food composition can be used for weight loss management, for the maintenance of a normal healthy body mass index, for the treatment and prevention of being overweight, and for the treatment and prevention of obesity.

Provided is a method for managing weight by administering to an individual in need thereof a functional food composition as described herein. The functional food composition can be administered in an amount sufficient for the maintenance of a normal body mass index. The functional food composition can be administered in an amount sufficient to decrease body mass index.

Provided is a method for the treatment of obese body mass index by administering to an individual in need thereof a functional food composition as described herein.

Provided is a method for the treatment of overweight body mass index by administering to an individual in need thereof a functional food composition as described herein.

This disclosure also relates to the delivery of compounds useful for skeletal health using edible or potable substances encapsulated in an edible membrane matrix. In particular aspects, the functional food provides functionally nutritive benefits for disease prevention and health wellness through the incorporation of certain biologically active compounds in the edible substance and/or as particles in the membrane matrix.

Provided herein is a functional food composition and method for making, comprising an edible or potable substance, a cross-linked matrix encapsulating the edible or potable substance, and a compound that promotes skeletal health. The functional food composition can comprise a compound that promotes (e.g., in the form of edible particles) skeletal health selected from the group consisting of a calcium compound, a magnesium compound, vitamin K₂, a calcium uptake enhancer, and derivatives thereof. The calcium uptake enhancer can be selected from the group consisting of colecalciferol and ergocalciferol. The calcium uptake enhancer can be vitamin D. The vitamin D concentration can be from about 1000 to about 4000 IU's, the vitamin K₂ concentration from about 50 micrograms to about 500 micrograms, the magnesium concentration from about 40 to about 320 milligrams, and the calcium concentration from about 500-2000 milligrams, in an individual functional food composition serving.

The functional food composition can comprise additional edible particles in the cross-linked matrix. The edible particles for skeletal health and/or additional edible particles can provide enhanced performance to the matrix.

The functional food composition edible particles for skeletal health and/or additional edible particles can have a size with a volume mean distribution between about 0.1 microns and about 1.0 microns, between about 0.1 microns and about 10.0 microns, between about 0.1 microns and about 100.0 microns, between about 0.1 microns and about 1.0 millimeters, or between about 0.1 and about 3 millimeters.

Provided herein is a method of preparing a functional food composition, comprising the steps of providing an edible or potable substance, an edible unpolymerized matrix, and a compound that promotes skeletal health, combining the edible or potable substance and/or the edible unpolymerized matrix with the compound that promotes skeletal health, and encapsulating the edible substance with the edible matrix. The method can further comprise the step of polymerizing the edible matrix.

The functional food composition can be used for maintenance of skeletal health, the prevention of rickets, the prevention of osteoporosis, the prevention of osteomalacia, to increase bone density, and for the prevention and treatment of cardiovascular disease.

Provided is a method for maintaining or improving skeletal health by administering to an individual in need thereof a functional food composition as described herein. The functional food composition can be administered in an amount sufficient to increase bone density in an individual. The functional food composition can be administered in an amount sufficient to decrease chronic joint and skeletal pain.

Provided is a method for the prevention and treatment of a skeletal disease by administering to an individual in need thereof a functional food composition as described herein. The skeletal disease can consist of at least one of rickets, osteoporosis, and osteomalacia.

This disclosure also relates to the delivery of bioflavonoids, carotenes and essential fatty acid compounds using edible or potable substances encapsulated in an edible membrane matrix. In particular aspects, the functional food provides functionally nutritive benefits for cardiovascular disease prevention and health wellness through the incorporation of certain biologically active compounds in the edible substance and/or as particles in the membrane matrix.

Provided herein is a functional food composition and method for making, comprising an edible or potable substance, a cross-linked matrix encapsulating the edible or potable substance, and a cardiovascular health compound comprising a bioflavonoid, a carotene, and an essential fatty acid.

The functional food composition can include a bioflavonoid derived from at least one of the group consisting of Camellia sinensis, Theobroma cacao, varieties thereof, strains thereof, cultivars thereof, and combinations thereof.

The functional food composition can include bioflavonoid selected from the group consisting of esveratrol, quercetin, rutin, catechin, epicatechin, proanthocyanidin, cocoaflavanol, and combinations thereof.

The functional food composition can comprise an essential fatty acid selected from the group consisting of α-linolenic acid, eicosapentanoic acid, docosahexaenoic acid, and combinations thereof.

The functional food composition can comprise carotene selected from the group consisting of α-carotene, β-carotene, γ-carotene, δ-carotene, ε-carotene, lycopene, and combinations thereof.

The functional food composition can comprise additional edible particles in the cross-linked matrix. The cardiovascular health compound and/or additional edible particles can provide enhanced performance to the matrix.

The functional food composition of the cardiovascular health compound and/or additional edible particles can have a size with a volume mean distribution between about 0.1 microns and about 1.0 microns, between about 0.1 microns and about 10.0 microns, between about 0.1 microns and about 100.0 microns, between about 0.1 microns and about 1.0 millimeters, or between about 0.1 and about 3 millimeters.

Provided herein is a method of preparing a functional food composition, comprising the steps of providing an edible or potable substance, an edible matrix, and a cardiovascular health compound comprising a bioflavonoid, a carotene, and an essential fatty acid, combining the edible or potable substance and/or the edible matrix with the cardiovascular health compound, and encapsulating the edible substance with the edible matrix. The method can further comprise the step of polymerizing the edible matrix.

The functional food composition can be used for the maintenance of cardiovascular health, for treatment of cardiovascular disease, for treatment of high blood pressure, and for treatment of high blood cholesterol.

Provided is a method for the maintenance of cardiovascular health by administering to an individual in need thereof a functional food composition as described herein.

Provided is a method for the treatment of cardiovascular disease by administering to an individual in need thereof a functional food composition as described herein.

Provided is a method for the treatment of high blood pressure by administering to an individual in need thereof a functional food composition as described herein.

Provided is a method for the treatment of high blood cholesterol by administering to an individual in need thereof a functional food composition as described herein.

This disclosure also relates to the delivery of compounds useful for joint and cartilage health using edible or potable substances encapsulated in an edible membrane matrix. In particular aspects, the functional food provides functionally nutritive benefits for disease prevention and health wellness through the incorporation of certain biologically active compounds in the edible substance and/or as particles in the membrane matrix.

Provided herein is a functional food composition and method for making, comprising an edible or potable substance, a cross-linked matrix encapsulating the edible or potable substance, and particles for joint and cartilage health. The joint and cartilage health compound may be glucosamine, chondroitin, collagen, hyaluronic acid, salts thereof, complexes thereof, bioavailable derivatives thereof, and bioactive derivatives thereof.

The functional food composition can consist of about 500 milligrams to about 2000 milligrams chondroitin, about 500 milligrams to about 2000 milligrams of glucosamine, about 20 to about 200 milligrams hyaluronic acid, or about 500 milligrams to about 3000 milligrams of collagen.

The functional food composition can include at least one ingredient selected from the group consisting of a vitamin, a mineral, an amino acid, an antioxidant, an anti-inflammatory agent, and an essential fatty acid.

The functional food composition can comprise additional edible particles in the cross-linked matrix. The edible particles for joint and cartilage health and/or additional edible particles can provide enhanced performance to the matrix.

The functional food composition can comprise edible particles for joint and cartilage health and/or additional edible particles can have a size with a volume mean distribution between about 0.1 microns and about 1.0 microns, between about 0.1 microns and about 10.0 microns, between about 0.1 microns and about 100.0 microns, between about 0.1 microns and about 1.0 millimeters, or between about 0.1 and about 3 millimeters.

Provided herein is a method of preparing a functional food composition, comprising the steps of providing an edible or potable substance, an edible unpolymerized matrix, and edible particles for joint and cartilage health, combining the edible or potable substance and/or the edible unpolymerized matrix with the edible particles for joint and cartilage health, and encapsulating the edible substance with the edible matrix. The method can further comprise the step of polymerizing the edible matrix.

The functional food composition can be used for maintenance of joint and cartilage health, the treatment and prevention of arthritis, the treatment and prevention of osteoarthritis, the treatment and prevention of rheumatoid arthritis, the treatment and prevention of psoriatic arthritis, the treatment and prevention of joint effusion, the treatment and prevention of joint pain, the treatment and prevention of joint inflammation, the treatment and prevention of synovitis, and for post-operative care.

Provided is a method for maintaining or improving joint and cartilage health by administering to an individual in need thereof a functional food composition as described herein. The functional food composition can be administered in an amount sufficient to decrease chronic joint pain in an individual.

Provided is a method for the prevention and treatment of a degenerative joint disease by administering to an individual in need thereof a functional food composition as described herein. The degenerative joint disease can consist of at least one of the group consisting of arthritis, joint effusion, and deterioration of proper joint function, and the arthritis can consist of at least one of the group selected from osteoarthritis, rheumatoid arthritis, and psoriatic arthritis.

Provided is a method for the prevention and treatment of a joint disorder by administering to an individual in need thereof a functional food composition as described herein. The joint disorder can consist of at least one of the group selected from joint inflammation, chronic joint pain, and synovitis.

This disclosure also relates to the delivery of multivitamin compounds using edible or potable substances encapsulated in an edible membrane matrix. In particular aspects, the functional food provides functionally nutritive benefits for disease prevention and health wellness through the incorporation of certain biologically active compounds in the edible substance and/or as particles in the membrane matrix.

Provided herein is a functional food composition and method for making, comprising an edible or potable substance, a cross-linked matrix encapsulating the edible or potable substance, and a multivitamin compound. The functional food composition can include a multivitamin wherein the multivitamin compound is at least two of the group consisting of vitamin A, vitamin B₁, vitamin B₂, vitamin B₃, vitamin B₅, vitamin B₆, vitamin B₉, vitamin B₁₂, vitamin C, vitamin D₃, vitamin E, vitamin K₁, vitamin K₂, vitamin H (biotin), and combinations thereof.

The functional food composition can be a multivitamin compound further comprising minerals. The functional food composition mineral can be at least one of the group selected from boron, calcium, chloride, chromium, cobalt, copper, fluorine, iodine, iron, magnesium, manganese, molybdenum, nickel, phosphorous, potassium, selenium, silicon, sodium, strontium, sulfur, vanadium, zinc, and combinations thereof.

The functional food composition can further comprise at least one of the group consisting of an amino acid, an antioxidant, a carotenoid, a flavonoid, an isoflavone, a nutraceutical, and combinations thereof.

The functional food composition can comprise additional edible particles in the cross-linked matrix. The multivitamin compound and/or additional edible particles can provide enhanced performance to the matrix.

The functional food composition edible multivitamin compound and/or additional edible particles can have a size with a volume mean distribution between about 0.1 microns and about 1.0 microns, between about 0.1 microns and about 10.0 microns, between about 0.1 microns and about 100.0 microns, between about 0.1 microns and about 1.0 millimeters, or between about 0.1 and about 3 millimeters.

Provided herein is a method of preparing a functional food composition, comprising the steps of providing an edible or potable substance, an edible matrix, and a multivitamin compound, combining the edible or potable substance and/or the edible matrix with the multivitamin compound, and encapsulating the edible substance with the edible matrix. The method can further comprise the step of polymerizing the edible matrix.

The functional food composition can be used for maintenance of recommended daily allowance of vitamins and minerals, for the treatment of vitamin and mineral deficiency related to dietary imbalance, for the treatment of vitamin and mineral deficiency related to chronic disease, for the treatment of vitamin and mineral deficiency related to the treatment of a chronic disease, for the treatment of vitamin and mineral deficiency related to a weight loss diet.

Provided is a method for the treatment of vitamin and mineral deficiency by administering to an individual in need thereof a functional food composition as described herein.

Provided is a method for the maintenance of recommended daily allowances of vitamins and minerals by administering to an individual in need thereof a functional food composition as described herein.

This disclosure also relates to the delivery of essential fatty acid compounds using edible or potable substances encapsulated in an edible membrane matrix. In particular aspects, the functional food provides functionally nutritive benefits for disease prevention and health wellness through the incorporation of certain biologically active compounds in the edible substance and/or as particles in the membrane matrix.

Provided herein is a functional food composition and method for making, comprising an edible or potable substance, a cross-linked matrix encapsulating the edible or potable substance, and an essential fatty acid compound. The functional food composition can include an essential fatty acid compound that is an essential fatty acid. The functional food composition essential fatty acid can be selected from the group consisting of α-linolenic acid, eicosapentanoic acid, docosahexaenoic acid, and combinations thereof.

The functional food composition can further comprise dietary fiber. The dietary fiber can be selected from the group consisting of galacto-oligosaccharides, inulin and oligofructose (fructo-oligosaccharide), isomalto-oligosaccharides, lactulose, lactosucrose, transgalacto-oligosaccharides, soybean oligosaccharides, tagatose, xylo-oligosaccharides, and combinations thereof.

The functional food composition can comprise additional edible particles in the cross-linked matrix. The essential fatty acid compound, dietary fiber, and/or additional edible particles can provide enhanced performance to the matrix.

The functional food composition essential fatty acids, dietary fiber, and/or additional edible particles can have a size with a volume mean distribution between about 0.1 microns and about 1.0 microns, between about 0.1 microns and about 10.0 microns, between about 0.1 microns and about 100.0 microns, between about 0.1 microns and about 1.0 millimeters, or between about 0.1 and about 3 millimeters.

Provided herein is a method of preparing a functional food composition, comprising the steps of providing an edible or potable substance, an edible unpolymerized matrix, and an essential fatty acid compound, combining the edible or potable substance and/or the edible unpolymerized matrix with the essential fatty acid compound, and encapsulating the edible substance with the edible matrix. The method can further comprise the step of polymerizing the edible matrix.

The functional food composition can be used for promoting normal development of the brain, eyes and nerves, for maintenance of normal development of the brain, eyes and nerves, for maintenance of brain, eye and nerve health, for the prevention and treatment of coronary disease, for the prevention and treatment of dementia, for the prevention and treatment of depression, and for the prevention and treatment of arthritis.

Provided is a method for promoting normal development and health maintenance of the brain, eyes and nerves by administering to an individual in need thereof a functional food composition as described herein.

Provided is a method for the treatment of coronary disease by administering to an individual in need thereof a functional food composition as described herein.

Provided is a method for the maintenance of coronary health by administering to an individual in need thereof a functional food composition as described herein.

Provided herein is a method for the prevention and treatment of dementia by administering to an individual in need thereof a functional food composition as described herein.

Provided is a method for the prevention and treatment of depression by administering to an individual in need thereof a functional food composition as described herein.

Provided is a method for the prevention and treatment of arthritis by administering to an individual in need thereof a functional food composition as described herein.

This disclosure also relates to the delivery of branched chain amino acids using edible or potable substances encapsulated in an edible membrane matrix. In particular aspects, the functional food provides functionally nutritive benefits for consumers by contributing to the treatment and prevention of certain diseases and disorders and the maintenance of general health and wellness through the incorporation of certain biologically active compounds in the edible substance and/or as particles in the membrane matrix.

Provided herein is a functional food composition and method for making, comprising an edible or potable substance, a cross-linked matrix encapsulating the edible or potable substance, the cross-linked matrix comprising an edible polymer, and a branched chain amino acid.

The functional food composition can further comprise L-glutamine. The functional food composition branched chain amino acid can be selected from the group consisting of L-leucine, L-isoleucine, L-valine, and combinations thereof.

The functional food composition can further comprise a multivitamin composition.

The functional food composition can comprise additional edible particles in the cross-linked matrix. The branched chain amino acid, L-glutamine, and/or additional edible particles can provide enhanced performance to the matrix.

The functional food composition of the branched chain amino acid, L-glutamine, and/or additional edible particles can have a size with a volume mean distribution between about 0.1 microns and about 1.0 microns, between about 0.1 microns and about 10.0 microns, between about 0.1 microns and about 100.0 microns, between about 0.1 microns and about 1.0 millimeters, or between about 0.1 and about 3 millimeters.

Provided herein is a method of preparing a functional food composition, comprising the steps of providing an edible or potable substance, an edible matrix, and a branched chain amino acid, combining the edible or potable substance and/or the edible matrix with the branched chain amino acid, and encapsulating the edible substance with the edible matrix. The method can further comprise the step of polymerizing the edible matrix.

The functional food composition can be used for the maintenance of muscle mass, to increase muscle mass, for the prevention and treatment of muscle mass loss during post-operative care, for the prevention and treatment of muscle mass loss during trauma care, for the prevention and treatment of muscle mass loss from malignant disease, and for the prevention and treatment of muscle mass loss from burn care.

Provided is method for the maintenance of muscle mass by administering to an individual in need thereof a functional food composition as described herein.

Provided is a method to increase muscle mass by administering to an individual in need thereof a functional food composition as described herein.

Provided is a method for the prevention and treatment of muscle mass loss during post-operative care by administering to an individual in need thereof a functional food composition as described herein.

Provided is a method for the prevention and treatment of muscle mass loss during trauma care by administering to an individual in need thereof a functional food composition as described herein.

Provided is a method for the prevention and treatment of muscle mass loss from malignant disease by administering to an individual in need thereof a functional food composition as described herein.

Provided is a method for the prevention and treatment of muscle mass loss from burn care by administering to an individual in need thereof a functional food composition as described herein.

This disclosure also relates to the delivery of antioxidants using edible or potable substances encapsulated in an edible membrane matrix. In particular aspects, the functional food provides functionally nutritive benefits for consumers by contributing to the treatment and prevention of certain diseases and disorders and the maintenance of general health and wellness through the incorporation of certain biologically active compounds in the edible substance and/or as particles in the membrane matrix.

Provided herein is a functional food composition and method for making, comprising an edible or potable substance, a cross-linked matrix encapsulating the edible or potable substance, the cross-linked matrix comprising an edible polymer and an edible antioxidant containing compound characterized by oxygen radical absorbance capacity above about 1000 per 100 grams.

The functional food composition can include edible particles for at least one of the group consisting of a bioflavonoid, a carotene, a vitamin, and combinations thereof, wherein the bioflavonoid is selected from the group consisting of esveratrol, quercetin, rutin, catechin, epicatechin, proanthocyanidin, and combinations thereof, wherein the carotene is selected from the group consisting of α-carotene, β-carotene, γ-carotene, δ-carotene, ε-carotene, lycopene, and combinations thereof, and wherein the vitamin is selected from the group consisting of vitamin A, vitamin C, vitamin E, and combinations thereof.

The functional food composition can further comprise a dietary fiber.

The functional food composition can comprise additional edible particles in the cross-linked matrix. The edible antioxidant containing compound compound and/or additional edible particles can provide enhanced performance to the matrix.

The functional food composition cross-linked matrix can have a thickness from about 10 microns to about 200 millimeters.

The functional food composition of the edible antioxidant containing compound and/or additional edible particles can have a size with a volume mean distribution between about 0.1 microns and about 1.0 microns, between about 0.1 microns and about 10.0 microns, between about 0.1 microns and about 100.0 microns, between about 0.1 microns and about 1.0 millimeters, or between about 0.1 and about 3 millimeters.

Provided herein is a method of preparing a functional food composition, comprising the steps of providing an edible or potable substance, an edible unpolymerized matrix, and particles of an edible antioxidant compound, wherein the edible antioxidant containing compound is characterized by an oxygen radical absorbance capacity above about 1000 per 100 grams, combining the edible or potable substance and/or the edible unpolymerized matrix with the edible antioxidant containing compound, and encapsulating the edible substance with the edible matrix. The method can further comprise the step of polymerizing the edible matrix.

The functional food composition can be used for the maintenance of skin health, for the maintenance of nails, for the maintenance of hair, for the prevention and treatment of dry skin, for the prevention and treatment of skin wrinkles, for the prevention and treatment of skin spots, for the prevention and treatment of brittle nails, for the prevention and treatment of hair loss, for the maintenance of cardiovascular health, for the treatment of cardiovascular disease, for the maintenance of brain function and mental acuity, for the maintenance of vision health, for the maintenance of healthy blood sugar levels, and for the maintenance of urinary tract health.

Provided is a method for the maintenance of cardiovascular health by administering to an individual in need thereof a functional food composition as described herein.

Provided is a method for the treatment of cardiovascular disease by administering to an individual in need thereof a functional food composition as described herein.

Provided is a method for the maintenance of skin health by administering to an individual in need thereof a functional food composition as described herein.

Provided is a method for the maintenance of hair health by administering to an individual in need thereof a functional food composition as described herein.

Provided is a method for the maintenance of nail health by administering to an individual in need thereof a functional food composition as described herein.

Provided is a method for the prevention and treatment of hair loss by administering to an individual in need thereof a functional food composition as described herein.

Provided is a method for the prevention and treatment of brittle nails by administering to an individual in need thereof a functional food composition as described herein.

Provided is a method for the prevention and treatment of skin wrinkles by administering to an individual in need thereof a functional food composition as described herein.

Provided is a method for the prevention and treatment of skin spots by administering to an individual in need thereof a functional food composition as described herein.

Provided is a method for the prevention and treatment of high blood cholesterol by administering to an individual in need thereof a functional food composition as described herein.

Provided is a method for the maintenance of brain function and mental acuity by administering to an individual in need thereof a functional food composition as described herein.

Provided is a method for the maintenance of vision health by administering to an individual in need thereof a functional food composition as described herein.

Provided is a method for the maintenance of healthy blood sugar levels by administering to an individual in need thereof a functional food composition as described herein.

Provided is a method for the maintenance of urinary tract health by administering to an individual in need thereof a functional food composition as described herein.

This disclosure also relates to functional food compositions. Functional food compositions are designed to deliver sufficient quantities and quality of β-glucans and probiotics using edible or potable substances encapsulated in an edible membrane matrix. In particular aspects, the functional food composition provides nutritive and health benefits for immunological response, disease treatment and prevention, and health maintenance through the incorporation of certain biologically active compounds in the edible substance and/or as particles in the membrane matrix.

Provided herein is a functional food composition and methods for making, comprising an edible or potable substance, a cross-linked matrix encapsulating the edible or potable substance, a probiotic, and β-glucan. The matrix can comprise the β-glucan and the edible or potable substance component comprises the probiotic, or the matrix comprises the probiotic and the edible or potable substance comprises the β-glucan. The matrix can comprise the probiotic and the β-glucan, or the edible or potable substance can comprise the probiotic and the β-glucan.

The functional food composition can comprise a probiotic concentration from about 1.0 to about 100 billion colony forming units in an individual edible transport vessel, and the probiotic can be an organism selected from the genera group consisting of Aspergillus, Bacillus, Bacteroides, Bifidobacterium, Brettanomyces, Enterococcum, Kluyveromyces, Lactobacillus, Lactococcus, Leuconostoc, Pediococcus, Propionibacterium, Saccharomyces, Shewanella, Streptococcus, Torulaspora, Vagococcus, and any derivatives, strains, and combinations thereof.

The functional food composition can comprise a probiotic that is heat killed. The heat killed probiotic can be in the functional food in amounts of about 100 mg to about 500 mg.

The functional food composition can comprise a β-glucan where the β-glucan is (1,3)-(1,6)-β-glucan. The β-glucan can be (1,3)-β-glycosidic linked D-glucose subunits β-(1,6)-linked to (1,3)-β-glycosidic linked D-glucose side chains of varying length and bonded to the backbone subunits at irregular or regular intervals. The β-glucan concentration can be from about 1.0 milligrams to 5.0 grams.

The functional food composition can comprise additional edible particles in the cross-linked matrix. The β-glucan, the probiotic, and/or additional edible particles can provide enhanced performance to the matrix.

The β-glucan, the probiotic, and/or additional edible particles can comprise a size having a volume mean distribution between about 0.1 microns and about 1.0 microns, between about 0.1 microns and about 10.0 microns, between about 0.1 microns and about 100.0 microns, between about 0.1 microns and about 1.0 millimeters, or between about 0.1 and about 3 millimeters.

The functional food composition can be used for increasing beneficial immune responses, for decreasing adverse immune responses, for maintaining healthy immune responses, for enhancement of a vaccination response, to stimulate antibody production, to reduce the occurrence of symptomatic common cold infections, for treating fungal infections, for treating Candida albicans infections, for a free-radical scavenger, for treatment of cancer, and for protection against radiation exposure.

Provided is a method for maintaining or improving immune health by administering to an individual in need thereof a functional food composition as described herein. The functional food composition can be administered in an amount sufficient to stimulate antibody production, for enhancement of a vaccination response, to reduce the occurrence of symptomatic common cold infections, for treating fungal infections, for treating Candida albicans infections, for use as a free-radical scavenger, for treatment of cancer, for protection against radiation.

In all functional food compositions, the matrix can be comprised of an edible polymer charge cross-linked by multivalent ions, including cross-linking interactions between the edible particles and edible polymer or plurality of edible polymers via bridges formed by the multivalent ions. The matrix can comprise a polysaccharide selected from the group consisting of a hydrocolloid, shellac, and fibers.

The cross-linked matrix of the functional food composition can have a thickness from about 10 microns to about 200 millimeters.

The functional food composition can have an edible or potable substance comprising at least one of a powder, a gel, an emulsion, a foam, a solid, and combinations thereof. The edible or potable substance can be selected from the group consisting of fruit, vegetable, meat, a dairy product, a carbohydrate food product, a botanical, a confection, and combinations thereof.

The functional food composition can have an edible or potable substance comprising a liquid, particularly wherein the liquid comprises at least one of water, an alcohol, a juice, an alcohol mixed drink, a coffee product, a tea product, a soft drink, an energy supplement product, a dietary supplement, a confection, fortified substances thereof, and combinations thereof.

The functional food composition can have an edible or potable substance defining a volume of between about 5 and 1,250 cubic centimeters, and the cross-linked matrix has a strength sufficient to contain and transport a liquid water volume equivalent to the interior volume defined by the edible or potable substance.

### DESCRIPTION OF DRAWINGS

FIG. 1 shows the chemical structure of an alginate polymer -(M)ₘ-(G)ₙ- (M: mannuronate; G: guluronate).

FIG. 2 illustrates polymerization of sodium alginates via divalent cations (e.g., Ca²⁺).

FIG. 3 is a schematic illustrating bonding between positive particles (e.g., Ca⁺ or Mg²⁺) and negative particles (e.g., alginate or food particles).

FIG. 4 illustrates multiple transport systems arranged in shells.

FIG. 5 illustrates a transport system having large particles suspended in an outer membrane layer.

FIG. 6 illustrates a transport system having small particles suspended in an outer membrane layer.

FIG. 7 illustrates a transport system having both large and small particles suspended in an outer membrane layer.

FIG. 8 illustrates a transport system having an outer membrane layer that is non-uniformly shaped.

FIGS. 9A-9C illustrate a multimembrane composition with particles embedded in the membrane and a particulate layer between membranes.

### DETAILED DESCRIPTION

### Definitions

B-glucan refers generally to chains of D-glucose polysaccharides linked by beta-type glycosidic bonds.

(β-1,3):(β-1,6) D-glucan and (1,3)-(1,6)-β-glucan refer to glucose polysaccharides consisting of a backbone of (1,3)-β-glycosidic linked D-glucose subunits β-(1,6)-linked to (1,3)-β-glycosidic linked D-glucose side chains of varying length and bonded to the backbone subunits at irregular or regular intervals.

“Bioavailable” and “bioavailability” refer to the quantity, fraction or proportion of the administered or ingested substance capable of being absorbed and available for use or storage, and varies according to physiological state and nutritional status of a consumer.

“Bioflavonoid” refers to flavones, isoflavanoids and neoflavanoids, sub-classes, sub-groups, and chemically related compounds thereof, including flavanols such as catechins and epicatechins, as used and defined according to IUPAC nomenclature and designation.

“Biologically active compound” and “biologically active agent” refer to any compound that elicits a response in or from a biological system and/or organism.

“Body Mass Index” refers to a measure of body fat based on height and weight that applies to adult men and women, wherein the quantitative measure is used to categorize an adult's weight as underweight, normal weight, overweight, and obese.

“Branched chain amino acid” refers to the essential amino acids L-leucine, L-isoleucine, and L-valine, separately or in combination.

“Elastic tissue component” refers to protein compounds derived from or located in skin, bone, connective tissue such as cartilage, tendons and ligaments, and/or other fibrous tissues as found in vertebrates, including, without limitation, collagen and elastin, that are characterized by increased tensile strength and/or elasticity.

“Essential fatty acid” refers to omega-3 fatty acids that cannot be synthesized de novo by the human body, and includes α-linoleic acid (ALA), eicosapentaenoic acid (EPA), and docosahexaenoic acid (DHA). It is known that diets including the shorter-chained omega-3 fatty acid ALA, will allow for the human body to form long-chain omega-3 fatty acid EPA and then from EPA, the most crucial, DHA, but this process has low efficiency.

“Fortified” or “fortify” refers to the adding of ingredients to something or to add further ingredients to food or drink in order to improve its flavor or add nutrients.

“Functional food” refers to an edible composition fortified with known biologically active compounds or agents that provide enhanced benefit (e.g. a health benefit, nutritional benefit) to the consumer. The enhanced benefit can be quantitative, qualitative or subjective and include for example, health promotion and disease prevention. In general, the functional food is fortified with defined (qualitative or quantitative) amounts of the known beneficial ingredients to provide the desired benefit.

“Integument” refers to skin and protein based skin cell products thereof, including hair and finger/toe nails.

“Matrix” refers to the molecular three dimensional architecture of a polymerized or unpolymerized polymer or copolymeric substance.

“Matrices” refers to two or more of a distinct matrix.

“Membrane” as used herein refers to the polymerized polymers and/or co-polymers encapsulating an edible or potable substance.

“Microbiota” refers to the collective term for microflora (i.e., any type of minute organism) that may be found within a given environment.

“Microflora” refers to living microscopic microorganisms that maintain a more or less constant presence in a particular area of living biological system, and includes bacteria, viruses, protozoa, yeasts and fungi.

“Nutraceutical” refers to a product isolated or purified, often from foods, that is generally sold in medicinal forms not usually associated with food, includes dietary supplements and food-related products for promoting health.

“ORAC” refers to a method of determining oxygen radical absorbance capacity. Data from the ORAC method shows antioxidant capacity of biologically active compounds in vitro.

“Probiotic” refers to living microorganisms that, on ingestion by a host organism, and in sufficient concentration, exert health benefits to the host organism beyond basic nutrition.

“Prebiotic” refers to a selectively fermented ingredient that allows specific changes, both in the composition and/or activity in the gastrointestinal microbiota that confers benefits upon host well-being and health.

“Shells” refers to relatively unpliable to hard polymerized polymers or co-polymers.

“Skins” as used herein refers to the polymerized polymers and/or co-polymers encapsulating an edible or potable substance.

I. Overview of the Prebiotic/Probiotic Functional Food Composition

The disclosure relates to functional food compositions that contain a prebiotic and a probiotic when, upon ingestion, promote gastrointestinal health and well-being. The functional food composition contains an edible or potable substance that is encapsulated in an edible membrane, and further contains a probiotic and a prebiotic. The functional food compositions described herein provide a convenient and improved way for individuals to consume probiotics in quantities needed to promote gastrointestinal health and well-being.

Generally, functional food compositions are intended to provide a healthy benefit to a consumer. The benefit can be qualitative, quantitative or subjective and include, for example, health promotion, disease prevention, etc. Some benefits include providing an important nutritive food source, vitamins and supplements, or probiotics, which contributes to the prevention, management and treatment of chronic diseases or disorders. This health improvement benefit can also be provided to a consumer when additional ingredients or more of an existing ingredient are added to a food so that the benefit from the food product is gained. However, delivery systems for some bioavailable compounds including prebiotics and probiotics often reduce their viability, resulting in suboptimal quantities of these compounds being made available to the consumer.

The functional food compositions described herein provide for a significant improvement in the viability and concentration of probiotics available to a consumer. Within these compositions, the prebiotic compounds and probiotic compounds are compartmentalized so that the edible or potable substance and the encapsulating membrane do not both contain the probiotic or both contain the prebiotic. Upon mastication and ingestion, the probiotic and the prebiotic are released from the functional food composition, and combined in the intestinal tract wherein the prebiotic provides an immediately available nutrition source for the probiotic to grow. Prebiotics and probiotics can thus be engineered and matched to each other to provide an optimal condition for quick population in the gut upon consumption by a host.

The functional food compositions described herein are designed to provide prebiotics and probiotics in biologically effective quantities superior to commonly used products. The compositions are particularly well-suited for providing a larger active ingredient load as well as being amenable for controlling the actual portion size for an intended health benefit. An eight ounce serving of commercial yogurt in plastic containers commonly provides a microbial load of less than 1 billion colony forming units (BCFU), far below a bacterial load level sufficient to provide health benefits. The compositions described herein can be designed to provide up to 100 BCFU's.

These functional food compositions also enhance the stability, gustatory experience, and delivery of functional biologically active compounds. Functional food compositions contain and protect ingestible/edible substances, such as ice cream, yogurts, etc. and probiotics or prebiotics, by encapsulating the substance within edible or biodegradable membranes. The edible membranes of these compositions can be formed from various natural polymeric substances allowing different compositions to be easily transported and consumed.

Considerations for the choice of probiotic or probiotic combinations for use in the function food compositions are that the probiotic remain viable until ingestion in a biologically effective concentration, and the probiotic be able to confer a health benefit to and/or use for treatment of a gastro-intestinal, immune or other systemic disorder in the host organism. In all instances, it is desirable that these food compositions (product)) include effectual probiotics to re-establish healthy intestinal bacterial flora or otherwise confer a health benefit to a host organism ingesting the probiotic.

Important variables when specifically considering a prebiotic and a probiotic for use in the functional food composition include: 1) the interaction between the prebiotic and probiotic, 2) the by-product left by the probiotic, 3) the taste and/or texture of the edible composition after the prebiotic and probiotic interact, 4) the rate of replication of the probiotic, and 5) the health benefit(s) the probiotic and/or prebiotic may convey.

In general, the prebiotic and the probiotic are substantially separated from each other so that the matrix component and the edible or potable substance component contain the prebiotic or the probiotic. If the matrix component includes the prebiotic, the edible or potable substance component can include the probiotic. If the matrix component includes the probiotic, the edible or potable substance component can include the prebiotic. In some applications, the location of the prebiotic and the probiotic may be conditionally dependent on each other. The effectiveness of the prebiotic or probiotic may be optimized based on placement within either the matrix or the edible or potable composition or they may be equally as effective in either location.

In general, the prebiotic and probiotic are separated in the composition so there is no interaction between the two compounds, and are designed to be released from the food product after or upon consumption. After consuming the serving of functional food composition, the prebiotic may be broken down by the body's natural microflora which could provide a better suited environment for the additional probiotics delivered by the edible product. The prebiotic and probiotic may interact after the edible composition is broken down and before the edible composition reaches the gastrointestinal tract (e.g., chewing, etc.). Mastication and ingestion release the components from the membrane matrix and the edible or potable substance to allow them mix, interact to allow for growth, and to colonize in the gut in sufficient amounts.

If desired, the functional food compositions can be prepared so that the prebiotic and probiotic are allowed to interact within the food product by diffusion from the membrane to the encapsulated food or vice versa. In these instances, the physical and/or chemical characteristics of the membrane can change as the prebiotic is used by the probiotic. As the prebiotic is used by the probiotic over time, the membrane can be altered in terms of its physical and/or chemical characteristics, for example, permeability, elasticity, color, taste, tensile strength, texture, pH, overall charge, etc. The changes occurring with the edible composition can be an indicator (e.g., the matrix displays a noticeable color change to indicate a sufficient number of probiotics has been reached, etc.). In some embodiments, the consumption of the prebiotic by the probiotic leaves behind by-products that affect the mouth feel or user experience of the edible composition (e.g., carbonation, etc.). In some embodiments, the by-products left in the edible product by the probiotic confer separate health benefits and/or alter the physical and/or chemical characteristics of the edible composition.

In some applications, the matrix pre- or probiotic component may diffuse into the edible or potable product while the edible or potable substance pre- or probiotic component remains in the edible or potable substance. Similarly, the edible or potable substance pre- or probiotic component can diffuse into the matrix while the matrix pre- or probiotic component remains in the matrix. After diffusion, the matrix pre- or probiotic component and the edible or potable substance pre- or probiotic component may interact. The edible matrix also can affect the diffusion rate between the probiotic microorganism and the prebiotic nutrient source for the microorganism based on type of material, charge, subunit composition, and/or embedded particles. If the probiotic diffuses out of the membrane, it may implement its valuable properties on the surrounding environment (e.g., the digestive tract, etc.). The matrix can be selectively permeable for prebiotic and/or probiotic diffusion while the edible product remains encapsulated and substances outside of the embodiment are repelled.

It may be desirable that the prebiotic and probiotic remain in their respective locations within the edible composition, but the probiotic may access the embedded prebiotic at the matrix-edible or potable substance interface and the probiotic may flourish within its respective location. The probiotic may use the prebiotic entirely, partially, or not at all while embodied within the same edible composition. If the prebiotic is in the edible composition in excess for the probiotic, the prebiotic may act as nutritional support for both the probiotic and the consumer.

Health benefit aspects contemplated for use of the pre- and probiotic functional food compositions described herein include relief from intestinal inflammation, pathogen related diarrhea, antibiotic related diarrhea, various irritable bowel syndromes, urogenital infections, allergies, lactose intolerance, colon cancer, high cholesterol, high blood pressure, immunity function, immune based infections, peptic ulcers, inflammation, colitis, necrotizing enterocolitis, Crohn's Disease, eczema, Inflammatory Bowel Disease, vitamin production, etc. Health and wellness preventative treatment with probiotics includes decreased intestinal gas and bloating, increased fecal transit time, relief from constipation, improved digestion, increased flora count in stool, etc. For example, probiotics may beneficially affect the host by augmenting the host intestinal microbial population beyond the amount already existing, thus possibly inhibiting unhealthy or harmful pathogens. Furthermore, improvement of these and other gastrointestinal disorders may raise the overall quality of life and daily functional capacity by increasing the regularity of bowel movements and easing pain related to certain disorders.

In general, prebiotics are included in the membrane and/or the edible or potable substance to further promote probiotic activity. The three criteria used for prebiotic classification are resistance to digestion, fermentation availability by intestinal microflora, and the selective stimulation of the growth and/or activity of intestinal bacteria associated with health and wellbeing. The prebiotics described herein are fortified within the functional food composition, in concentration and/or type, to be in addition to natural levels of a prebiotic in the food source.

As described in more detail herein, the common probiotics include lactic acid bacteria, bifidobacteria, yeasts and bacilli, and are often consumed as part of a fermented food source, (e.g., yogurt, soy yogurt, etc.) with added live microbiological cultures.

Measurable outcomes for general health maintenance can be determined quantitatively and/or qualitatively by monitoring, for example, a patient's or consumer's change in composition of intestinal microflora, possibly feelings of well-being, and bowel regularity after a program or regimen of daily consumption of the functional food composition is established. For pretreatment/prevention of diseases, conditions and/or syndroms, the consumer or patient may show milder symptoms or no symptoms. In acute conditions for which the consumer or patient is using the functional food composition, for example diarrheal conditions (i.e. traveler's, antibiotic or viral), the outcome would be faster resolution and reduction in severity for treatment. Chronic conditions such as irritable bowel syndrome, inflammatory bowel disease, Crohn's Disease, etc., a patient or consumer would show measurable reduction in number of episodes and severity of diarrhea and or constipation. In general, a reduction in abdominal pain and discomfort, improvement in quality of life and ability to perform activities of daily living, measurable differences in frequency of episodic conditions and evaluation of changes in concentration of gastrointestinal microflora are all indicators of the health benefits when consuming the functional food compositions for gastrointestinal health described herein.

Prebiotic and Probiotic Compositions

Common probiotics include lactic acid bacteria, bifidobacteria, yeasts and bacilli, and are often consumed as part of a fermented food source, (e.g., yogurt, soy yogurt, etc.) with added live microbiological cultures. Probiotics in concentration and/or type contained within the functional food compositions included herein may be used in addition to natural levels of probiotics in the food source.

Probiotics for use in the foods described herein include, but are not limited to, Aspergillus niger, Aspergillus oryzae, Bacillus coagulans, Bacillus lentus, Bacillus licheniformis, Bacillus pumilus, Bacillus subtilis, Bacteroides amylophilus, Bacteroides capillosus, Bacteroides ruminocola, Bacteroides suis, Bifidobacterium animalis, Bifidobacterium bifidum, Bifidobacterium breve, Bifidobacterium infantis, Bifidobacterium licheniformis, Bifidobacterium longum, Bifidobacterium pseudo longum, Bifidobacterium subtilus, Bifidobacterium thermophilum, Enterococcus cremoris, Enterococcus diacetylactis, Enterococcus faecium, Enterococcus thermophilus, Lactobacillus acidophilus, Lactobacillus amylovorus, Lactobacillus ansporogenes, Lactobacillus brevis, Lactobacillus buchneri, Lactobacillus bulgaris, Lactobacillus casei, Lactobacillus caucasicus, Lactobacillus cellobiosus, Lactobacillus crispatus, Lactobacillus curvatus, Lactobacillus delbrueckii, Lactobacillus farciminis, Lactobacillus fermentum, Lactobacillus gallinarum, Lactobacillus gasseri, Lactobacillus GG, Lactobacillus helveticus, Lactobacillus johnsonii, Lactobacillus kefir, Lactobacillus lactis, Lactobacillus plantarum, Lactobacillus reuteri, Lactobacillus rhamnosus (casei), Lactobacillus salivarius, Lactococcus lactis, Leuconostoc mesenteroides, Pediococcus acidilactici, Pediococcus acidilacticii, Pediococcus cerevisiae, Pediococcus pentosaceus, Propionbacterium freudenreichii, Propionibacterium shermanii, Saccharomyces boulardii, Saccharomyces cerevisiae, Shewanella colwelliana, Shewanella olleyana, Shewanella putrefaciens, Streptococcus cremoris, Streptococcus faecium, Streptococcus infantis, Streptococcus thermophilus, Vagococcus fluvialis, Bacillus coagulans GBI-30, 6086, Bifidobacterium animalis DN 173 010, Bifidobacterium animalis subsp. lactis BB-12, Bifidobacterium breve Yakult, Bifidobacterium infantis 35624, Bifidobacterium lactis HN019 (DR10), Bifidobacterium longum BB536, Bifidobacterium longum subsp. infantis 35624, Enterococcus LAB SF 68, Escerichia coli Nissle 1917, Lactobacillus acidophilus NCFM, Lactobacillus acidophilus LA-5, Lactobacillus acidophilus NCFM, Lactobacillus casei subspecies casei, Lactobacillus casei DN-114 001, Lactobacillus casei CRL431, Lactobacillus casei F19, Lactobacillus casei Shirota, Lactobacillus delbrueckii subspecies bulgaricus, Lactobacillus delbrueckii subspecies lactis, Lactobacillus paracasei St11 (or NCC2461), Lactobacillus johnsonii La1 (Lj1), Lactobacillus johnsonii NCC533, Lactobacillus plantarum 299v, Lactobacillus rhamnosus ATCC 53013 (LG), Lactobacillus rhamnosus LB21, Lactobacillus reuteri ATCC 55730, Lactobacillus salivarius (UCC118), Lactobacillus reuteri SD2112, Lactococcus lactis L1A, Lactococcus lactis subspecies lactis, Lactococcus lactis subspecies lactis biovariant diacetylactis, Lactococcus lactis subspecies cremoris, Leuconostoc mesenteroides subspecies cremoris, Leuconostoc mesenteroides subspecies dextranicum, Lactobacillus reuteri Protectis (DSM 17938, daughter strain of ATCC 55730), Saccharomyces cerevisiae boulardii, Saccharomyces cerevisiae boulardii (lyo), Lactobacillus bulgaricus, Streptococcus thermophiles, Bifidobacterium spp, and any other strains, derivatives, and combinations thereof, including, but not limited to, Lactobacillus rhamnosus GR-1 & Lactobacillus reuteri RC-14, Lactobacillus acidophilus CL 1285 & Lactobacillus casei Lbc80r, Lactobacillus rhamnosus (ATT SD5675) & Lactobacillus acidophilus (STCC SD5221), VSL #3 (a combination of 1 strain of Streptococcus thermophiles, four strains of Lactobacillus species, & three Bifidobacterium species strains from Sigma-Tau Pharmaceuticals), Lactobacillus acidophilus NCFM & Bifidobacterium bifidum BB-12, Lactobacillus acidophilus CUL60 & Bifidobacterium bifidum CUL20, Lactobacillus acidophilus CL1285 & Lactobacillus casei LBC8OR, Lactobacillus helveticus R0052 & Lactobacillus rhamnosus R0011, Bacillus clausii (strains O/C, NR, SIN and T), Lactobacillus plantarum HEAL 9 & Lactobacillus paracasei 8700:2, other genetic variants thereof, and combinations and other combinations thereof.

When the dosage or intake of the effective dose of the active ingredients of the functional food composition are indicated by the number (concentration) of the probiotic, it is preferable that the intake is about 0.5-1.0 (BCFU) or more per day, more preferably about 10 BCFU's or more per day, more preferably about 50 BCFU's or more per day, more preferably about 100 BCFU's or more per day. The concentration of probiotic to be contained in an individual product serving is determined according to the amount of servings ingested per day. The concentration of probiotic present in the functional food composition can be less than about 1 BCFU's, about 1 to about 2 BCFU's, about 3 to about 5 BCFU's, about 5 to about 10 BCFU's, about 10 to about 20 BCFU's, about 20 to about 30 BCFU's, about 30 to about 40 BCFU's, about 40 to about 50 BCFU's, about 50 to about 60 BCFU's, about 60 to about 70 BCFU's, about 70 to about 80 BCFU's, about 80 to about 90 BCFU's, about 90 to about 100 or more BCFU's.

Under these criteria, the prebiotics can include, but are not limited to, galacto-oligosaccharides, inulin and oligofructose (fructo-oligosaccharide), isomalto-oligosaccharides, lactulose, lactosucrose, transgalacto-oligosaccharides, soybean oligosaccharides, tagatose, xylo-oligosaccharides, and combinations thereof.

The prebiotic can be an oligosaccharide fructan including, but not limited to, fructo-oligosaccharide and derivatives thereof of inulin (poly-D-fructose, itself derived from various plant products including bananas, onions, chicory root, garlic, asparagus, barley, wheat, jicama, leeks, etc.) or from transfructosylation action of a β-fructosidase as used, for example, by Aspergillus sp. metabolically active on sucrose. In other embodiments, the prebiotics are derived from plants and plant products including, but not limited to, artichoke, dandelion greens, bran, flour, legumes, oats, citrus fruits, apples, and root vegetables, cane sugar, etc. If desired, the prebiotic also serves as a dietary fiber source for the consumer or patient, but in certain conditions may be depleted away from being a nutritional source for the probiotic.

Compositions described herein include prebiotics and probiotics for maintenance and treatment of gastrointestinal health. If desired, additional compounds are included in the functional food composition to improve or maintain healthy conditions in the digestive tract. Additional compounds can be insoluble fibers and/or soluble fibers generally not specifically utilized by the probiotic; probiotic extracts (e.g., probiotic exudates and/or non-viable micro-organisms); digestive enzymes including, but not limited to, pancreatin, pancrelipase, papain, pepsin, diatase, ox bile, lactase, protease, amylase, lipase, bromelain, cellulose, malt diastase, glucoamylase, hemicellulose, beta-gluconase, phytase, trypsin, aminopeptidase, chymotrypsin, carboxypeptidase, elastase, maltase, sucrose, etc.; supplements including, but not limited to, glutamine, peppermint, artichoke, ginger, fennel, licorice root, anise seed; and compounds useful for immune health including, but not limited to, vitamins (A, E, C, D); minerals (zinc, selenium); plant derived materials such as Echinacea, ashwaganda, elderberry, etc.; and yeast extracts.

Uses of Prebiotic/Probiotic Functional Foods

Edible transport compositions described herein are contemplated to provide a functional benefit to a consumer (“functional food”). Functional foods are formulated to provide an important nutritive food source that contributes to the prevention, management and/or treatment of chronic diseases. This benefit can be provided to a consumer when new ingredients or more of an existing ingredient are added to a food so that an added health benefit from the food product is gained. Probiotics are useful for a variety of health related conditions as well as general health maintenance. The effectiveness of probiotic treatment is enhanced through the use of a prebiotic that is consumed with a probiotic. The edible transport compositions as described herein increase the effective delivered concentration of such useful probiotics, and can be formulated for daily dietary supplements, preventative treatment or treatment of nosocomial infections, or for clinical applications treating systemic disease states.

Often the probiotics are recommended by physicians and nutritionists to treat, for example, candidiasis, infections by opportunistic bacteria establishing after trauma, stress, excessive alcohol, exposure to toxins, lactose intolerance, or the establishment of normal flora after treatment with antibiotics. In some embodiments, the edible transport compositions are used to assist the consumer's natural flora to re-establish and/or maintain normal concentrations in the stool and the digestive tract. Often the probiotics are recommended by physicians and nutritionists to treat, for example, candidiasis, infections by opportunistic bacteria establishing after trauma, or the establishment of normal flora after treatment with antibiotics. In some embodiments of the edible transport compositions, maintenance and/or treatment of gastrointestinal conditions or disorders is provided through a regimen of daily consumption at high concentrations (50-100 BCFU's) for one to two weeks followed by a maintenance treatment in a normal diet (1-10 BCFU's) to assist in the re-establishment normal health conditions for a consumer. Maintenance treatment in a normal daily diet (1-10 BCFU's) can be useful for increasing transit times of stool though the intestinal tract, decreasing intestinal gas production and bloating, increasing microflora levels in the stool, and/or improving digestion. Other conditions for which clinical changes and mitigation of intestinal symptoms are desired can take longer treatments at higher concentrations of the prebiotic and/or probiotic, for example 4-8 weeks.

Probiotics may play a role in the prevention of certain types of cancer (e.g. colon cancer) due to their ability to bind and metabolize carcinogenic substances such as heterocyclic amines. Lower rates of colon cancer have been correlated with high consumption of fermented dairy products. In some embodiments of the edible transport compositions, maintenance and/or treatment is provided through a daily regimen (1-10 BCFU's), which can be useful for contributing to the prevention of certain cancers.

Probiotics may play a role in benefitting the immune system by, for example, competing against pathogens though competitive inhibition (e.g. opportunistic bacteria such as C. difficile), enhancing phagocytosis by white blood cells, and enhancing or increasing the production of certain antibodies and/or T-lymphocytes. Modulation of the immune system by regulating inflammatory and hypersensitivity responses related to cytokine regulation and function is also found to be correlated to intake of probiotics, as is the management of diarrhea and inflammatory bowel disease. In some embodiments of the edible transport compositions, maintenance and/or treatment of immune response conditions or disorders is provided through a regimen of daily consumption at high concentrations (50-100 BCFU's) for one to two weeks followed by a maintenance treatment in a normal diet (1-10 BCFU's) to assist in the re-establishment of normal health conditions for a consumer. Maintenance treatment in a normal daily diet (1-10 BCFU's) can be useful for management of diarrhea and inflammatory bowel disease.

Probiotics may play a role in the treatment for systemic disorders such as high cholesterol and high blood pressure. Ingestion of sufficient levels of probiotics is correlated with lowering cholesterol through a mechanism thought to be involved with the breakdown of bile in the intestinal tract to a metabolite that is unable to be reabsorbed. Additionally, reduction in blood pressure has been correlated with intake of fermented dairy products. Thus, it is believed that angiotensin-converting-enzyme inhibitors or inhibitor like peptides are produced by the probiotic. Maintenance treatment in a normal daily diet (1-10 BCFU's) can be useful for management of systemic disorders such as high cholesterol and/or high blood pressure.

II. Overview of the Functional Nutrition Compositions for Integument Health

The disclosure relates to functional food compositions that contain compounds when, upon ingestion, provide nutritional supplements and an increase in general health, beauty and well-being. The functional food composition contains an edible or potable substance that is encapsulated in an edible membrane, and further contains an integument health compound comprising bioflavonoids, carotenes and an elastic tissue component supplementing a normal diet. The functional food compositions described herein provide a convenient and improved way for individuals to consume bioflavonoids, carotenes and an elastic tissue component in combinations and/or sufficient quantities needed to supplement normal diet, promote and/or maintain integument health, treat conditions particularly related to integument health, and increase general health, beauty and well-being related thereto.

Generally, functional food compositions are intended to provide a healthy benefit to a consumer. The benefit can be qualitative, quantitative or subjective and include, for example, health maintenance and promotion, disease prevention, etc. Some benefits arise from providing an important nutritive food source, vitamins and supplements and/or compounds such as bioflavonoids, carotenes and elastic tissue components that generally or specifically contribute to increased health and beauty. A higher degree of health assists in the prevention, management and treatment of acute or chronic diseases or disorders. This health and beauty improvement or benefit can be provided to a consumer when additional ingredients or more of an existing ingredient are added to a food so that the benefit from the food composition is gained. However, delivery systems for some bioavailable compounds, including those important for supplementing a diet, can reduce the viability of the compounds, resulting in suboptimal quantities of these compounds being made available to the consumer for a particular food composition. Often, the delivery system for a nutritional supplement is in a form insufficient to promote compliance in a particular consumer group (e.g., elderly, infirm, children and young adults), such as being in a large pill form, powder form with little gustatory appeal, or poor tasting liquids.

The functional food compositions described herein provide for a significant improvement in the delivery, viability and concentration of bioflavonoids, carotenes and elastic tissue components useful to a consumer. Within these compositions, the bioflavonoids, carotenes and elastic tissue components can be compartmentalized within the edible or potable substance, the encapsulating membrane, or both. Upon mastication and ingestion, the bioflavonoids, carotenes and elastic tissue components are released from the functional food composition to provide an immediately available nutrition and edible source. The functional food compositions can thus be designed to have one or more bioflavonoids, carotenes and elastic tissue components in an effective combination and concentration desirable for the specific needs of the consumer or patient, for example, the elderly, adult and children consumers, and medical patients.

The functional food compositions described herein are designed to provide bioflavonoids, carotenes and elastic tissue components in biologically effective quantities for promoting integument health superior to commonly used products. The compositions are particularly well-suited for providing a larger active ingredient load as well as being amenable for controlling the actual portion size for an intended health benefit. Functional food compositions as described herein can provide up to and above several times the suggested minimum dosages in a sample serving, or provide combinations of bioflavonoids, carotenes, elastic tissue components, nutraceuticals and supplements up to and above several times the suggested minimum dosages in a single serving that is both convenient and appetizing. A functional food delivering a combination of integument health compounds in a single serving composition can result in the cessation or mitigation of a multitude of skin related conditions (e.g., wrinkles, dry skin, brittle nails, hair loss, skin spots, etc.), supplement dietary imbalance, and promote user compliance in the daily consumption of these and other health promoting compounds. In addition, a general state of beauty, health and wellness can result from a healthy diet supplemented with these and other health promoting compounds.

Population segments that may find useful a bioflavonoids, carotenes and elastic tissue component functional food composition as described herein include the elderly, adults, hospital patients, and those with a variety of skin conditions such as wrinkles, dryness, etc.

Consumers and patients can become noncompliant for daily intake of health compounds depending on the delivery form of the composition. For example, some health compounds are in pill form with a flavor, texture and overall size (e.g., omega-3 and omega-6 fatty acids, multivitamin supplements, etc.) that often result in reluctance to consume on a scheduled or daily basis. Therefore, a functional food as described herein that delivers a daily concentration of integument health compounds can overcome non-compliance.

The functional food compositions described herein also enhance the stability, gustatory experience, and delivery of functional biologically active compounds. The functional food compositions contain and protect ingestible/edible substances, such as ice cream, yogurts, etc., in addition to bioflavonoids, carotenes and elastic tissue components, by encapsulating the substance within edible or biodegradable membranes, for which the membranes provide barriers to oxygen and oxygen degradation, diffusion, etc. The edible membranes of these compositions can be formed from various natural polymeric substances that allow the compositions to be easily transported and consumed by a consumer.

Various bioflavonoids, carotenes, elastic tissue components, supplements, and nutraceuticals can be included in the functional food compositions described herein in quantities sufficient to correlate with recommended daily allowances, for the maintenance of integument health, treatment and prevention of integument disorders and related conditions, and are beneficial supplementations to normal dietary intake.

Aging, metabolic and hormonal changes related to aging, exposure to environmental conditions (sun exposure, arid environments), etc., can all contribute to changes in skin appearance and health. Metabolic changes, although natural, can result in decreased production of collagen and/or elastin that affect the overall appearance and texture of skin, nails and hair. Environmental exposure such as ultraviolet (UV) radiation also damages skin cells and hair. Over the longer term, UV radiation induces degenerative changes in cells of the skin, fibrous tissue and blood vessels leading to premature skin aging and wrinkles, lentigo, etc. UV exposure directly affects the bilayer making up a cell membrane because it is made up of a significant amount of unsaturated lipids that are more prone to attack by free radicals. Damage by free radicals is assumed to be one of the causative factors of aging, inflammation and hyper pigmentation. In fact, the stratum corneum has been found to produce significant concentrations free radicals under UV exposure, damaging DNA, lipids, proteins and carbohydrates. Nitric oxide and reactive oxygen species (ROS) including superoxide anion and hydrogen peroxide are highly reactive oxidant molecules produced by the human body through normal metabolism and diet, and also may play a role in certain conditions including the loss of elasticity in skin. Antioxidants are protective compounds that inactivate ROS and delay or prevent oxidative damage.

Lycopene is a natural carotene found in tomato, and functions at least as an antioxidant or oxygen scavenger. It is a powerful antioxidant and anti-inflammatory, with activity twice as high as that of β-carotene and tenfold higher than α-tocopherol, that can aid in protecting integument from environmental agents as well as contribute to healthy maintenance of texture and appearance. Studies suggest that ingestion of lycopene contributes to increased protection from sunburn, and that higher concentrations of lycopene in cutaneous skin layers enhance a softer skin texture. Therefore, a functional food composition rich in lycopene can contribute to more pleasant skin texture, youthful skin appearance, beauty and the mitigation of free radical damage.

Medical investigations provide that regular consumption of flavonoid-containing foods also can reduce the risk of oxidative damage as is found from skin damage or aged appearance of the integument. While bioflavonoids are ubiquitous in plants, cocoa (Theobroma cacao) and tea (Camellia sinensis) have been found to be particularly rich in a sub-class of flavonoids known as flavanols. A number of human dietary intervention trials with flavanol-containing cocoa and tea products have demonstrated improvements in systemic function from conditions related to oxidative damage. Flavanols are also found in fruits and vegetables and are linked to certain health benefits. As with lycopene other carotenes, flavanols are thought to take part in mechanisms such as nitric oxide and antioxidant, anti-inflammatory, and antiplatelet effects to improve endothelial function, lipid levels, particularly with respect to preventing the oxidization of unsaturated lipids. Therefore, a functional food product rich in bioflavanoids can contribute to mitigating the effect of free radical damage on the integument, youthful skin appearance, beauty, and maintenance of integument health and well-being.

Collagen is a group of naturally occurring proteins found in the integument and connective tissues of animals, making up about one third of the whole-body protein content. Collagen occurs in many places throughout the body and comprises the main component of connective tissue. Many types of collagen are known, the most common being collagen I (integument, tendon, vascular ligature, organs, the main component of the organic part of bone), collagen II (main component of cartilage), collagen III (main component of reticulate fibers and commonly found alongside type I), collagen IV (forming the bases of cell basement membrane), and collagen V (cell surfaces, hair and placenta). Collagen-related diseases most commonly arise from genetic defects or nutritional deficiencies that affect the biosynthesis, assembly, post-translational modification, secretion, or other processes involved in normal collagen production. Hydrolyzed and non-hydrolyzed type II collagen, often in combination with other joint and cartilage health compounds, can be used for joint pain relief and treatment of arthritis and other joint conditions and diseases. Elastin is a protein in connective tissues that provides mechanical strength and elastic character, allowing tissues to resume a resting state shape after stretching or contracting. Elastin is a significant component particularly in the skin. As both collagen and elastin are significant elastic tissue components of the skin, a functional food product rich in elastin and collagen may contribute to more pleasant skin texture, youthful skin appearance, beauty, and maintenance of integument health and well-being.

The functional food compositions with bioflavonoids, carotenes and elastic tissue components described herein include, but are not limited to, bioavailable forms of bioflavonoids, carotenes and elastic tissue components, bioactive derivatives thereof, salts thereof, and bioactive metabolic intermediates.

Other compounds can be used exclusively or in combination with bioflavonoids, carotenes and elastic tissue components of the functional food compositions described herein. Nutraceuticals are generally thought of as food or food product that reportedly provide health and medical benefits, including the prevention and treatment of disease, and can be defined as a product isolated or purified from foods that is generally sold in medicinal forms not usually associated with food. A nutraceutical may have a physiological benefit or provide protection against chronic disease. Such nutraceutical products may range from isolated nutrients, vitamins and minerals, other dietary supplements, and herbal products. With recent developments in cellular-level nutraceutical agents, researchers, and medical practitioners are developing templates for integrating and assessing information from clinical studies on complementary and alternative therapies into responsible medical practice.

Functional Food Compositions with Bioflavonoids, Carotenes and Elastic Tissue Compounds for Integument Health and Maintenance

In general antioxidants contemplated for use for the functional food composition as described herein can include, without limitation carotenes, bioflavonoids (flavonoids, isoflavones, neoflavonals), tocopherol, tocotrienol, lipoic acid, melatonin, superoxide dismutase, coenzyme Q10, alpha lipoic acid, vitamin A, chromium biotin, selenium and ascorbic acid. Carotenes contemplated for use as membrane particles include α-carotene, β-carotene, γ-carotene, δ-carotene, ε-carotene, lycopene, cryptoxanthin, lutein, zeaxathin, apocarotenal astaxanthin, canthaxanthin, lutein/lutein esters, etc. If desired, the amount of carotene in the functional food composition serving as described herein can be up to about 1 milligram (mg), up to about 5 mg, up to about 10 mg, up to about 15 mg, up to about 20 mg, up to about 25 mg, up to about 30 mg, up to about 35 mg, up to about 35 mg, up to about 40 mg, up to about 45 mg, up to about 50 mg, up to about 60 mg, up to about 70 mg, up to about 80 mg, up to about 90 mg, up to about 100 mg. Bioflavonoid used for the functional food composition described herein include esveratrol, quercetin, rutin, catechin, epichatechins, proanthocyanidins, and combinations thereof. Bioflavonoids can be derived from various plants including, for example, Camellia sinensis, Theobroma cacao (cocoa flavanols) and species, varieties, cultivars, thereof. Bioflavonoids can also be derived from or found in various plant extracts including, but not limited to, acai berry extract, raspberry extract, cranberry extract, pomegranate extract, plum extract, cherry extract, rosemary extract, etc. Isoflavones are used as membrane particles, including, but not limited to, genistein, daidzein, biochanin A, and formononetin. If desired, the amount of bioflavonoid in the functional food composition serving as described herein can be up to about up to about 50 mg, up to about 100 mg, up to about 150 mg, up to about 200 mg, up to about 250 mg, up to about 300 mg, up to about 350 mg, up to about 400 mg, up to about 450 mg, up to about 500 mg, up to about 600 mg, up to about 700 mg, up to about 800 mg, up to about 900 mg, up to about 1000 mg.

Generally, collagen compounds comprising type II and/or hydrolyzed type II collagen and elastin, both commercially available, have been shown to be useful for maintenance and treatment of integument condition. If desired, the amount of collagen provided in an integument health functional food composition can be sufficient for a recommended daily intake, for example, up to 1000 mgs total, up to about 2000 mgs total, up to about 3000 mgs total, up to about 4000 mgs total, up to about 5000 mgs total, up to about 6000 mgs total, up to about 7000 mgs total, up to about 8000 mgs total, up to about 9000 mgs total, up to about 10,000 mgs. If desired, the amount of elastin provided in an integument health functional food composition can be sufficient for a recommended daily intake, for example, up to about 500 mgs, up to 1000 mgs total, up to about 2000 mgs total, up to about 3000 mgs total, up to about 4000 mgs total, up to about 5000 mgs total, up to about 6000 mgs total, up to about 7000 mgs total, up to about 8000 mgs total, up to about 9000 mgs total, up to about 10,000 mgs.

Uses of Functional Food Compositions for Integument Health

Functional food compositions described herein are contemplated to provide a functional benefit to a consumer (“functional food”). Functional foods are formulated to provide an important nutritive food source that contributes to the prevention, management and/or treatment of chronic diseases and/or maintenance of a healthy state, as well as provide a source of bioflavonoids, carotenes and elastic tissue components for integument health, treatment and maintenance. These benefits can be provided to a consumer when new ingredients or more of an existing ingredient are added to a food so that an added health benefit from the food product is gained.

Supplementing a diet with bioflavonoids, carotenes and elastic tissue components deliverable in the functional foods as described herein is useful for prevention and treatment of a variety of conditions and disorders, particularly those related to skin appearance (wrinkles, dryness, skin spots), brittle nails, hair loss, as well as for general health and beauty maintenance.

The functional food compositions as described herein increase the effective delivered concentration of such useful bioflavonoids, carotenes and elastic tissue components, and can be formulated for daily dietary supplements, preventative treatment of integument damage (UV light, aridity), and other disorders related to environmental damage and aging.

III. Overview of the Functional Nutrition Compositions for Weight Loss

The disclosure relates to functional food compositions that contain compounds when, upon ingestion, promote weight loss and an increase in general health and well-being. The functional food composition contains an edible or potable substance that is encapsulated in an edible membrane, and further contains compounds for promoting weight loss. The functional food compositions described herein provide a convenient and improved way for individuals to consume weight loss compounds in combinations and/or sufficient quantities needed to promote weight loss and an increase in general health and well-being related thereto.

Generally, functional food compositions are intended to provide a healthy benefit to a consumer. The benefit can be qualitative, quantitative or subjective and include, for example, health promotion, disease prevention, etc. Some benefits arise from providing an important nutritive food source, vitamins and supplements and/or compounds that generally or specifically contribute to increased health. A higher degree of health assists in the prevention, management and treatment of acute or chronic diseases or disorders. This health improvement benefit can be provided to a consumer when additional ingredients or more of an existing ingredient are added to a food so that the benefit from the food composition is gained. However, delivery systems for some bioavailable compounds, including those important for weight loss, can reduce the viability of the compounds, resulting in suboptimal quantities of these compounds being made available to the consumer for a particular food composition.

The functional food compositions described herein provide for a significant improvement in the delivery, viability and concentration of compounds useful for weight loss available to a consumer. Within these compositions, the weight loss compounds can be compartmentalized within the edible or potable substance, the encapsulating membrane, or both. Upon mastication and ingestion, the weight loss compounds are released from the functional food composition to provide an immediately available nutrition and edible source that promotes weight loss. The functional food compositions can thus be designed to have one or more weight loss compounds in an effective combination and concentration desirable for the specific needs of the consumer or patient, for example, overweight or obese elderly, adult and children consumers, and medical patients.

The functional food compositions described herein are designed to provide weight loss compounds in biologically effective quantities superior to commonly used products. The compositions are particularly well-suited for providing a larger active ingredient load as well as being amenable for controlling the actual portion size for an intended health benefit. Functional food compositions as described herein can provide up to and above several times the suggested minimum dosages in a simple serving, or provide combinations of weight loss compounds, vitamins and supplements up to and above several times the suggested minimum dosages in a single serving that is both convenient and appetizing. A functional food delivering a combination of vitamins, supplements and weight loss compounds in a single product can result in the cessation or mitigation of a multitude of health related conditions and diseases. In addition, a general state of health and wellness resulting from weight loss can lead to a better psychological presentation as social pressures, medical issues and financial implications of excess and persistent weight can lead to a general decline in happiness and well-being.

Population segments that may find useful a weight loss composition as described herein include the elderly, adults and youth in addition to hospital patients. These consumers and patients can become susceptible to high cholesterol and blood pressure, heart disease, diabetes, increased risk of stroke, heart attack and cancer, depression, bone and cartilage disease and disorders, gastrointestinal disorders, respiratory problems and systemic disorders including those related to nephrology and urology.

The functional food compositions described herein also enhance the stability, gustatory experience, and delivery of functional biologically active compounds. The functional food compositions contain and protect ingestible/edible substances, such as ice cream, yogurts, etc. and weight loss compounds, by encapsulating the substance within edible or biodegradable membranes, for which the membranes provide barriers to oxygen, diffusion, etc. The edible membranes of these compositions can be formed from various natural polymeric substances that allow the compositions to be easily transported and consumed by a consumer.

Various supplements, nutraceuticals and products for weight loss also can be included in the functional food compositions described herein in quantities sufficient to correlate with increased state of health and beneficial treatment of weight related disorders, in a form that provides a tasty and delicious gustatory experience. The functional food compositions for use in weight loss regimens and general nutrition described herein include, but are not limited to, bioavailable forms of appetite suppressants, acting to manipulate hormonal and chemical processes in the body that otherwise increase hunger and/or the sense of feeling satiated (e.g., anorectics such as epinephrine and norepinephrine/noradrenaline), fat or cholesterol uptake inhibitors (such as green tea extract), gastrointestinal fillers, and thermogeneic compounds which boost a normal metabolic rate of the individual and result in metabolism of fat stores. Weight loss compounds can be synthetic or natural.

Some compounds, for example thylakoids and thylakoid extracts, are known to elicit more than one effect, for example regulating enzymes important for appetite control (e.g., appetite signaling proteins including as ghreilin or satiety signaling proteins including leptin), steric interference by binding to fat molecules for uptake inhibition, attenuating glucose uptake, and as a gastrointestinal filler, all of which are contemplated for use in the present invention.

Generally, the weight loss functional food compositions as described herein can use thylakoid compounds and/or extracts. Ingestion of thylakoids results in the suppression of appetite, contributing to satiety, as well as contributing to binding of fat molecules. The net effect is the temporal delay of fat digestion in the gut and the triggering of natural satiety signals that affect feeding behavior and eating patterns. A regimen that includes thylakoid and/or thylakoid extracts can thus result in the induction of weight loss by the consumer.

Other compounds can be used exclusively or in combination with weight loss described herein for functional food compositions described herein. If desired, such weight loss compounds include, but are not limited to, satiety inducing compounds and fat uptake inhibitors such as proteinase inhibitor II, green coffee bean extract, chlorogenic acid, green tea leaf extract, polyphenols, ashwagandha extract, xanthum gum, and pinolenic acid, hoodia, chitosan, chromium picolinate, conjugated linoleic acid, glucomannan, green tea extract, guar gum, guarana, guggal, senna, ephedra, bitter orange, fucoxanthin, white bean extract, vitamin D, human chorionic gonadotropin, resveratrol, capsaicin, chia, hoodia, L-carnitine, raspberry ketones, banana leaf, red clover, ginger, almonds, acai berry, flax seeds, leucine, and lipodrene.

Additionally, weight loss functional foods as described herein may be fortified with additional supplements, minerals and/or nutrients, including essential amino acids, vitamins and minerals, anti-oxidants, and essential fatty acids.

In general, a reduction in weight through the consumption of weight loss functional foods as described herein can lead to mitigation and treatment of weight related disorders and diseases, improvement in quality of life and ability to perform activities of daily living, and measurable differences in various health indicators (including, for example, obesity, high cholesterol levels, high blood pressure, mitigation and cessation of adult-onset diabetes, etc.).

Weight Loss Compositions

Compounds in concentration and/or type contained within the functional food compositions included herein may be used in addition to natural levels of weight loss compounds in the food source.

Generally, thylakoids, thylakoid extracts and bioavailable and bioactive metabolic derivatives and/or complexes can be included in membranes encapsulating an edible or potable substance and/or within the edible or potable substance. Concentrated, isolated and purified thylakoid compositions and thylakoid extracts are commercially available. Certain thylakoid compositions and/or extracts are isolated from spinach leaves. However, any concentrated and/or purified thylakoids compounds and extracts from any plant or microbiological source (for example, cyanobacteria) are contemplated for use with these functional foods. If desired, the amount of purified thylakoid composition or extract provided in a weight loss functional food composition sufficient for an effective daily intake can be, for example, up to 1 gram (g) total, up to about 2.5 g total, up to about 5 g total, up to about 7.5 g total, up to about 10 total, up to about 12.5 g total, up to about 15 g total, up to about 17.5 g total, up to about 20 g total, up to about 22.5 g total, up to about 25 g total, up to about 27.5 g total, up to about 30 g total.

If desired, other weight loss compositions can be included in the functional food compositions described herein. Other compounds can include, without limitation, proteinase inhibitor II, green coffee bean extract, chlorogenic acid, green tea leaf extract, polyphenols, ashwagandha extract, xanthum gum, and pinolenic acid, hoodia, chitosan, chromium picolinate, conjugated linoleic acid, glucomannan, green tea extract, guar gum, guarana, guggal, senna, ephedra, bitter orange, fucoxanthin, white bean extract, vitamin D, human chorionic gonadotropin, resveratrol, capsaicin, chia, hoodia, L-carnitine, raspberry ketones, banana leaf, red clover, ginger, almonds, acai berry, flax seeds, leucine, and lipodrene.

Generally, other compounds and/or bioavailable metabolic derivatives can include vitamins (for example, vitamin A, vitamin B, vitamin C, vitamin D, vitamin E, vitamin K, etc), minerals (for example calcium, iron, copper, zinc, magnesium, manganese, phosphorus, potassium, etc.), essential fatty acids (for example, omega-3-fatty acid, omega-6-fatty acid, etc.), essential amino acids (L-histidine, L-isoleucine, L-leucine, L-lysine, L-methionine, L-phenylalanine, L-valine and L-threonine), antioxidant agents and anti-inflammatory agents.

Uses of Edible Compositions for Weight Loss and Target Weight Maintenance

Edible transport compositions described herein are contemplated to provide a functional benefit to a consumer (“functional food”). Functional foods are formulated to provide an important nutritive food source that contributes to the prevention, management and/or treatment of chronic diseases and/or maintenance of a healthy state. This benefit can be provided to a consumer when new ingredients or more of an existing ingredient are added to a food so that an added health benefit from the food product is gained. Weight loss compounds such thylakoids, thylakoid extracts, proteinase inhibitor II, green coffee bean extract, chlorogenic acid, green tea leaf extract, polyphenols, ashwagandha extract, xanthum gum, and pinolenic acid, hoodia, chitosan, chromium picolinate, conjugated linoleic acid, glucomannan, green tea extract, guar gum, guarana, guggal, senna, ephedra, bitter orange, fucoxanthin, white bean extract, vitamin D, human chorionic gonadotropin, resveratrol, capsaicin, chia, hoodia, L-carnitine, raspberry ketones, banana leaf, red clover, ginger, almonds, acai berry, flax seeds, leucine, and lipodrene are useful for prevention and treatment of a variety of conditions and diseases as well as general health maintenance. The effectiveness of weight loss treatment and healthy weight maintenance is enhanced through the use of an efficient delivery system. The functional food compositions as described herein increase the effective delivered concentration of such useful weight loss compositions, and can be formulated for daily dietary supplements, preventative treatment or for clinical applications treating systemic or chronic disease states and disorders.

Supplementing daily dietary intake with weight loss compounds as a component of the functional food composition contributes to maintenance of healthy weight or loss of excess weight. Persons of all ages potentially can find useful these compositions, to prevent weight gain or as part of a daily regimen for weight loss. Various diseases and conditions can present in a consumer or patient from obesity and being overweight, and thus are targeted for mitigation though consumption of these functional foods, including high cholesterol and blood pressure, heart disease, diabetes, increased risk of stroke, heart attack and cancer, depression, bone and cartilage disease and disorders, gastrointestinal disorders, respiratory problems and systemic disorders including those related to nephrology and urology.

Maintenance treatment in a daily diet with functional food compositions directed to general weight maintenance and diet (up to about 5 g thylakoid or thylakoid extract) can be useful for the prevention of weight gain or treatment for weight loss, and contribute to general health and wellness of the consumer. Ideal weights can be estimated from, for example, a Body Mass Index (BMI) calculation ([weight (pounds)/height² (inches)]×703). BMI ranges are given by, for example, the Centers for Disease Control, to provide guidance on ideal BMI and target weights that are not considered “obese” and “overweight.” Such BMI indicators indicating “overweight” an “obese” have been correlated to the onset of chronic diseases and disorders. Examples of BMI ranges include: Underweight is less than 18.5, normal weight is 18.5 to 24.9, overweight is 25 to 29.9, obese is greater than 30.

IV. Overview of the Functional Nutrition Compositions for Skeletal Health

The disclosure relates to functional food compositions that contain compounds when, upon ingestion, promote skeletal structure health and well-being and maintenance of bone structure. The functional food composition contains an edible or potable substance that is encapsulated in an edible membrane, and further contains compounds for skeletal structure health. The functional food compositions described herein provide a convenient and improved way for individuals to consume bone health compounds in combinations and/or sufficient quantities needed to promote skeletal structure health and well-being related thereto.

Generally, functional food compositions are intended to provide a healthy benefit to a consumer. The benefit can be qualitative, quantitative or subjective and include, for example, health promotion, disease prevention, etc. Some benefits arise from providing an important nutritive food source, vitamins and supplements and/or compounds that generally or specifically contribute to increased health. A higher degree of health assists in the prevention, management and treatment of acute or chronic diseases or disorders. This health improvement benefit can be provided to a consumer when additional ingredients or more of an existing ingredient are added to a food so that the benefit from the food composition is gained. However, delivery systems for some bioavailable compounds, including those important for skeletal health, can reduce the viability of the compounds, resulting in suboptimal quantities of these compounds being made available to the consumer for a particular food composition.

The functional food compositions described herein provide for a significant improvement in the delivery, viability and concentration of compounds useful for skeletal health and maintenance available to a consumer. Within these compositions, the skeletal health compounds can be compartmentalized within the edible or potable substance, the encapsulating membrane, or both. Upon mastication and ingestion, the skeletal health compounds are released from the functional food composition to provide an immediately available nutrition source. The functional food compositions can thus be designed to have one or more skeletal health compounds in an effective combination and concentration desirable for the specific needs of the consumer or patient, for example, elderly, children, and medical patients.

The functional food compositions described herein are designed to provide skeletal health compounds in biologically effective quantities superior to commonly used products. The compositions are particularly well-suited for providing a larger active ingredient load as well as being amenable for controlling the actual portion size for an intended health benefit. Functional food compositions as described herein can provide up to and above several times the suggested minimum dosages in a simple serving, or provide combinations of skeletal health compounds, vitamins and supplements up to and above several times the suggested minimum dosages in a single serving that is both convenient and appetizing. A functional food delivering a combination of vitamins, supplements and skeletal health compounds in a single product can benefit from a resulting synergistic interaction, as has been identified, for example, in patients having hormone therapy/replacement or when used by postmenopausal patients. Population segments that may find useful a functional food skeletal health composition as described herein include the eldery susceptible to osteoporosis, persons who may not receive a sufficient amount of sunlight to stimulate natural vitamin D production (darker skinned people, infants, pregnant women, etc.), children suffering from osteomalacia, and the like.

The functional food compositions described herein also enhance the stability, gustatory experience, and delivery of functional biologically active compounds. The functional food compositions contain and protect ingestible/edible substances, such as ice cream, yogurts, etc. and skeletal health compounds, by encapsulating the substance within edible or biodegradable membranes, for which the membranes provide barriers to oxygen, diffusion, etc. The edible membranes of these compositions can be formed from various natural polymeric substances that allow the compositions to be easily transported and consumed by a consumer.

Various supplements, nutraceuticals and products for skeletal health also can be included in the functional food compositions described herein in quantities sufficient to correlate with increased state of health and beneficial treatment of bone and skeletal structure disorders, in a form that provides a tasty and delicious gustatory experience. The functional food compositions for use in skeletal health and nutrition described herein include, but are not limited to, bioavailable forms of vitamin D and/or other calcium uptake enhancers, calcium/calcium salts, magnesium/magnesium salts, vitamin K compounds, etc.

Vitamin D (comprising colecalciferol and ergocalciferol) has long been known to be important for enhancing calcium and phosphorous uptake in the intestinal tract. Vitamin D is a fat-soluble vitamin that is naturally available in some foods and can be ingested in dietary supplement form. Humans can also produce vitamin D when ultraviolet rays from sunlight strike the skin to initiate vitamin D synthesis. Vitamin D is biologically inert and must undergo two hydroxylations in the body for activation (vitamin D to 25-hydroxyvitamin D (calcidiol) to 1,25-dihydroxyvitamin D (calcitriol), the latter being physiologically active). Vitamin D assists in intestinal calcium absorption for the maintenance of calcium and phosphate concentrations for skeletal health, bone growth and the prevention of, for example, hypocalcemic tetany. Without an adequate vitamin D concentration in the body, bones can become thin, brittle, or misshapen.

If desired for a particular application, the form of vitamin D and/or metabolic and bioactive/bioavailable derivatives thereof can be provided in the edible or potable substance, and/or in membranes as, for example, powdered form, micelles and/or as particulates. Inclusion of particles in the membrane can also contribute to performance enhancement (increased strength, preferable mouth-feel and flavor, permeability preferences, etc.) of the membrane.

Vitamin K was shown to have a positive impact on bone building, especially when taken with calcium and vitamin D. Vitamin K biologically activates osteocalcin, which enables calcium incorporation into the proper three-dimensional structure of bone. Vitamin K refers to a group of menaquinone compounds including Vitamin K₁, and Vitamin K₂ which is further comprised of, for example, short chain menaquinones (e.g., MK-4) and long chain menaquinones (e.g., MK-7, MK-8, and MK-9). Gla-proteins are dependent on Vitamin K proteins, and are synthesized in a variety of tissue types. In particular for bone health, osteocalcin is a non-collagenous protein secreted by osteoblasts and plays an essential role in the formation of mineral in bone and maintenance of bone mineral density, and may be particularly important as a supplement in menopausal women. Matrix Gla-protein is a calcification inhibitory protein also found in numerous body tissues, and has a pronounced role in cartilage maintenance.

There are two commonly recognized kinds of vitamin K deficiency: acute and chronic. Acute deficiency can be manifested as unusual bleeding from gums, nose, and/or the gastrointestinal tract. The results of acute deficiency are often severe and can include internal clogging of the blood vessels resulting in strokes, embolism, lung damage, and in severe cases, death caused by immoderate blood loss. Chronic vitamin K deficiency is less obvious than acute deficiency and potentially puts an individual at higher risk because there are no obvious symptoms. Chronic vitamin K deficiency can result in impairments in bone, cardiovascular health, and other disease of aging. Therefore, supplementing a diet with vitamin K in a functional food composition as described herein can contribute to proper development of bone, thereby decreasing, for example, the potential for bone fractures due to poor skeletal health, development of osteoporosis, and also contribute to improved cardiovascular health.

Calcium and magnesium are available in some foods, medicines, and as a dietary supplement. Almost all of the body's calcium supply is stored in the bones and teeth to support structure and function, whereas about half of the body's supply of magnesium is in the bones. Bone undergoes continuous restructuring and functions with constant resorption and deposition of calcium into new bone, with the extent of bone resorption/deposition changing depending on physical stresses and age. In older adults, the breakdown of bone exceeds resorption/deposition and can result in the development of osteoporosis. Additionally, calcium is required for other bodily and cellular functions including vascular contraction and vasodilation, muscle function, nerve transmission, intracellular signaling and hormonal secretion. Magnesium is needed for hundreds of biochemical reactions in the body, maintenance of normal muscle and nerve function, a healthy immune system, and bone integrity. Magnesium is also involved with blood sugar and blood pressure regulation, energy metabolism and protein synthesis. Generally, these and other compositions known to be necessary for bone health can be included in a functional food composition for increasing skeletal health.

Beneficial aspects of skeletal health that are commonly sought include increased bone density and strength, modulation of cell growth, neuromuscular and immune function, reduction of inflammation, and the regulation of certain enzyme activities. Functional food compositions specific to bone and skeletal structure contribute to disease prevention, for example rickets, osteomalacia, and osteoporosis, in addition to the general health and wellness of the skeletal support structure. Measurable outcomes for general health maintenance can be determined qualitatively and/or quantitatively by monitoring, for example, a patient's or consumer's change in bone density, blood level concentrations of vitamin K, vitamin D, calcium, and or magnesium, possible feelings of well-being, and decreasing levels of chronic joint and skeletal pain. For pretreatment/prevention of diseases, conditions and/or syndromes, the consumer or patient may show milder symptoms or no symptoms. In acute conditions for which the consumer or patient is using the functional food composition, for example acute vitamin K deficiency, the outcome would be faster resolution of symptoms (e.g., cessation of bleeding gums) and reduction in symptom severity during and after treatment. For chronic conditions such as osteoporosis or osteomalacia, a patient or consumer would show measurable increase in bone density and critical blood concentration of metabolites, for example, calcium and magnesium. In general, a reduction in pain and discomfort, improvement in quality of life and ability to perform activities of daily living, and measurable differences in concentration of metabolites or bone density are all indicators of the health benefits when consuming the functional food compositions for skeletal health described herein.

Skeletal Health Compositions

Compounds in concentration and/or type contained within the functional food compositions included herein may be used in addition to natural levels of skeletal health compounds in the food source.

Generally, vitamin D, its bioavailable and bioactive metabolic derivatives and/or complexes can be included in membranes encapsulating an edible or potable substance and/or within the edible or potable substance. If desired, the amount of vitamin D provided in a skeletal health functional food composition can be sufficient for a recommended daily intake, for example, up to about 500 International Units (IU), up to about 1000 IU, up to about 2000 IU, up to about 3000 IU, or up to about 4000 IU.

Generally, any one or combination of calcium compounds and/or salts are incorporated into the membrane and/or edible or potable substance, including, but not limited to, calcium acetate, calcium carbonate, calcium citrate, calcium citrate malate, calcium gluconate, calcium lactate, calcium lactogluconate, and tricalcium phosphate. If desired, a skeletal health functional food composition can provide up to about 200 mgs total, up to about 400 mgs total, up to about 600 mgs total, up to about 800 mgs total, up to about 1000 mgs total, up to about 1200 mgs total of one or more bioavailable calcium compounds.

Generally, magnesium compounds and/or salts are incorporated into the membrane and/or edible or potable substance, including magnesium ascorbate, magnesium aspartate, magnesium bicarbonate, magnesium carbonate, magnesium chloride, magnesium citrate, magnesium fumarate, magnesium gluconate, magnesium glutamate, magnesium glycinate, magnesium hydroxide, magnesium lactate, magnesium lysinate, magnesium malate, magnesium orotate, magnesium oxide, magnesium phosphate, magnesium pidolate, magnesium taurate, and magnesium sulfate (or magnesium sulphate). If desired, a skeletal health functional food composition can provide up to about 80 milligrams (mgs), up to about 100 mgs, up to about 120 mgs, up to about 140 mgs, up to about 160 mgs, up to about 180 mgs, up to about 200 mgs, up to about 220 mgs, up to about 240 mgs, up to about 260 mgs, up to about 280 mgs, up to about 300 mgs, up to about 320 mgs.

Generally, combinations of calcium compounds and/or salts, magnesium compounds and/or salts, and/or calcium uptake compounds can be included in the functional food composition as described herein. In general it is desirable that the form of calcium compounds and/or salts, magnesium compounds and/or salts, and/or calcium uptake compounds and/or bioavailable metabolic derivatives (e.g., in powdered form, micelles, etc.) is provided in the membranes and contributes to performance enhancement of the membrane. If desired, in addition to or alternatively to vitamin D as a calcium uptake compound, other calcium uptake compounds can be used in the functional food composition, including, but not limited to, calcitonin, albumin and parathyroid hormone (PTH), effectors of PTH release, and combinations, bioavailable and bioactive derivatives and/or metabolic intermediates of these compounds.

Generally, vitamin K₁ and/or vitamin K₂ compounds and/or bioavailable metabolic derivatives are incorporated into the membrane and/or edible or potable substance. In general it is desirable that the form of vitamin K and/or bioavailable metabolic derivatives (e.g., powdered form, micelles, etc.) is provided in the membranes and contributes to performance enhancement of the membrane. Some applications of the functional food compositions can provide up to about 5 micrograms, up to about 75 micrograms, up to about 100 micrograms, up to about 200 micrograms, up to about 300 micrograms, up to about 400 micrograms, up to about 500 micrograms of vitamin K₁ and/or vitamin K₂ compounds and/or bioavailable and bioactive metabolic derivatives.

Uses of Edible Compositions for Skeletal Health and Maintenance

Edible transport compositions described herein are contemplated to provide a functional benefit to a consumer (“functional food”). Functional foods are formulated to provide an important nutritive food source that contributes to the prevention, management and/or treatment of chronic diseases. This benefit can be provided to a consumer when new ingredients or more of an existing ingredient are added to a food so that an added health benefit from the food product is gained. Skeletal health related compounds or supplements such as Vitamin D, Vitamin K, calcium and magnesium are useful for a variety of conditions and diseases as well as general health maintenance. The effectiveness of skeletal health treatment and maintenance is enhanced through the use of an efficient delivery system. The functional food compositions as described herein increase the effective delivered concentration of such useful skeletal health compositions, and can be formulated for daily dietary supplements, preventative treatment or for clinical applications treating systemic disease states.

There are two commonly recognized kinds of vitamin K deficiency: acute and chronic. Acute deficiency can be manifested as unusual bleeding from gums, nose, and/or the gastrointestinal tract. The results of acute deficiency are often severe and can include internal clogging of the blood vessels resulting in strokes, embolism, lung damage, and in severe cases, death caused by immoderate blood loss. Infants are considered to be at an increased risk for acute vitamin K deficiency, because vitamin K is not transported sufficiently across the placenta. Chronic vitamin K deficiency is less obvious than acute deficiency and potentially puts an individual at higher risk because there are no obvious symptoms. Chronic vitamin K deficiency can result in impairments in bone, cardiovascular health, and other disease of aging.

Supplementing daily dietary intake with skeletal health compounds as a functional food contributes to maintenance of new bone, an important health concern that increases with physical stresses and age. In older adults, the breakdown of bone exceeds resorption/deposition and can result in the development of osteoporosis. Additionally, calcium is required for other bodily and cellular functions including vascular contraction and vasodilation, muscle function, nerve transmission, intracellular signaling and hormonal secretion. Magnesium is needed for hundreds of biochemical reactions in the body, maintenance of normal muscle and nerve function, a healthy immune system, and bone integrity. Magnesium is also involved with blood sugar and blood pressure regulation, energy metabolism and protein synthesis.

Maintenance treatment in a daily diet with functional food compositions directed to general bone health (up to 4000 IU vitamin D, 1000 mgs calcium, 100 micrograms vitamin K₂, and 320 mgs magnesium) can be useful for the prevention of bone loss, for example osteoporosis and osteomalacia, the prevention of disease states, for example rickets, mitigating chronic bone and joint pain, and contributing to general health and wellness of the skeletal support structure.

V. Overview of the Functional Nutrition Compositions for Cardiovascular Health

The disclosure relates to functional food compositions that contain compounds when, upon ingestion, provide nutritional supplements and an increase in general health and well-being. The functional food composition contains an edible or potable substance that is encapsulated in an edible membrane, and further contains bioflavonoids, carotenes and essential fatty acid compounds supplementing a normal diet. The functional food compositions described herein provide a convenient and improved way for individuals to consume bioflavonoids, carotenes and essential fatty acid compounds in combinations and/or sufficient quantities needed to supplement normal diet, promote normal growth and development, treat disease conditions particularly related to cardiovascular health, and increase general health and well-being related thereto.

Generally, functional food compositions are intended to provide a healthy benefit to a consumer. The benefit can be qualitative, quantitative or subjective and include, for example, health promotion, disease prevention, etc. Some benefits arise from providing an important nutritive food source, vitamins and supplements and/or compounds such as bioflavonoids, carotenes and essential fatty acid compounds that generally or specifically contribute to increased health. A higher degree of health assists in the prevention, management and treatment of acute or chronic diseases or disorders. This health improvement benefit can be provided to a consumer when additional ingredients or more of an existing ingredient are added to a food so that the benefit from the food composition is gained. However, delivery systems for some bioavailable compounds, including those important for supplementing a diet, can reduce the viability of the compounds, resulting in suboptimal quantities of these compounds being made available to the consumer for a particular food composition. Often, the delivery system for a nutritional supplement is in a form insufficient to promote compliance in a particular consumer group (e.g., elderly, infirm, children and young adults), such as being in a large pill form, powder form with little gustatory appeal, or poor tasting liquids.

The functional food compositions described herein provide for a significant improvement in the delivery, viability and concentration of bioflavonoids, carotenes and essential fatty acid compounds useful to a consumer. Within these compositions, the bioflavonoids, carotenes and essential fatty acid compounds can be compartmentalized within the edible or potable substance, the encapsulating membrane, or both. Upon mastication and ingestion, the bioflavonoids, carotenes and essential fatty acid are released from the functional food composition to provide an immediately available nutrition and edible source. The functional food compositions can thus be designed to have one or more bioflavonoids, carotenes and essential fatty acid in an effective combination and concentration desirable for the specific needs of the consumer or patient, for example, the elderly, adult and children consumers, and medical patients.

The functional food compositions described herein are designed to provide bioflavonoids, carotenes and essential fatty acid compounds in biologically effective quantities for promoting cardiovascular health superior to commonly used products. The compositions are particularly well-suited for providing a larger active ingredient load as well as being amenable for controlling the actual portion size for an intended health benefit. Functional food compositions as described herein can provide up to and above several times the suggested minimum dosages in a sample serving, or provide combinations of bioflavonoids, carotenes and essential fatty acids, nutraceuticals and supplements up to and above several times the suggested minimum dosages in a single serving that is both convenient and appetizing. A functional food delivering a combination of cardiovascular health compounds in a single serving composition can result in the cessation or mitigation of a multitude of cardiac health related conditions and diseases (e.g., high cholesterol, high blood pressure, etc.), supplement dietary imbalance, and promote compliance in the daily consumption of these and other health promoting compounds. In addition, a general state of health and wellness can result from a healthy diet supplemented with these and other health promoting compounds.

Population segments that may find useful a bioflavonoid, carotene and essential fatty acid functional food composition as described herein include the elderly, adults, hospital patients, those with cardiovascular disease, and those at risk for cardiovascular disease.

Consumers and patients can become noncompliant for daily intake of cardiovascular health compounds depending on the delivery form of the composition. For example, some cardiovascular health compounds are in pill form with a flavor, texture and overall size (e.g., omega-3 and omega-6 fatty acids) that often results in reluctance to consume on a scheduled or daily basis. Therefore, a functional food as described herein that delivers a daily concentration of cardiovascular health compounds can overcome non-compliance.

The functional food compositions described herein also enhance the stability, gustatory experience, and delivery of cardiovascular health compounds. The functional food compositions contain and protect ingestible/edible substances, such as ice cream, yogurts, etc., in addition to bioflavonoids, carotenes and essential fatty acid compounds, by encapsulating the substance within edible or biodegradable membranes, for which the membranes provide barriers to oxygen and oxygen degradation, diffusion, etc. The double bond(s) found within essential fatty acid compounds are particularly susceptible to oxidative degradation, and thus it is desirable to ensure protection against oxygen. The edible membranes of these compositions can be formed from various natural polymeric substances that allow the compositions to be easily transported and consumed by a consumer.

Various bioflavonoids, carotenes and essential fatty acid compounds, supplements, and nutraceuticals can be included in the functional food compositions described herein in quantities sufficient to correlate with recommended daily allowances, for the maintenance of cardiovascular health, prevention of cardiovascular diseases and beneficial supplementation to normal dietary intake.

Lower rates of cardiovascular disease are attributed in part to the so-called Mediterranean diet, which is rich in plant derived bioactive phytochemicals. Lycopene is a natural carotene found in tomato, an essential component of the Mediterranean diet, and functions at least as an antioxidant scavenger. Nitric oxide and reactive oxygen species (ROS) including superoxide anion and hydrogen peroxide are highly reactive oxidant molecules produced by the human body through normal metabolism and diet, and may play a role in certain diseases including cardiovascular diseases. Antioxidants are protective compounds that inactivate ROS and delay or prevent oxidative damage. Lycopene, is one of the most potent antioxidants, with activity twice as high as that of β-carotene and tenfold higher than α-tocopherol. The oxidization of low density lipoprotein (“bad cholesterol”) is thought to be the primary step leading to its interaction with macrophages inside the arterial wall and the formation of atherosclerotic plaque. Therefore higher levels of blood antioxidants are thought to contribute to mitigating the degree of low density lipoprotein oxidation and plaque formation.

Medical investigations provide that regular consumption of flavonoid-containing foods also can reduce the risk of cardiovascular diseases, providing direct evidence for the potential cardiovascular benefits. While bioflavonoids are ubiquitous in plants, cocoa (Theobroma cacao) and tea (Camellia sinensis) have been found to be particularly rich in a sub-class of flavonoids known as flavanols. A number of human dietary intervention trials with flavanol-containing cocoa and tea products have demonstrated improvements in systemic function as well as regulation in blood pressure. Flavanols are also found in fruits and vegetables and are linked to certain health benefits linked to coronary heart disease and stroke. As with lycopene and other carotenes, flavanols are thought to take part in mechanisms such as nitric oxide and antioxidant, anti-inflammatory, and antiplatelet effects to improve endothelial function, lipid levels, blood pressure and insulin resistance, particularly with respect to preventing the oxidization of low density lipoprotein.

Generally, essential fatty acids as used herein can be considered as supplementing naturally obtained essential fatty acids in food. Essential fatty acids (DHA, in particular) are consumed as part of a healthy diet in order to maintain and promote the treatment and/or prevention of cardiovascular disease and related disorders and events, such as high blood pressure, high cholesterol, the onset of arthrosclerosis, heart attack and stroke. Essential fatty acids are also important for the maintenance of organ systems including eyes, nerves, brain, etc. Essential fatty acids can also add to a higher quality or perceived quality of the health state of the end user, as some correlations have been identified with decrease in cancer risk, effectiveness against arthritis, dementia and depression.

The functional food compositions with bioflavonoids, carotenes and essential fatty acid compounds described herein include, but are not limited to, bioavailable forms of bioflavonoids, carotenes and essential fatty acid compounds, bioactive derivatives thereof, salts thereof, and bioactive metabolic intermediates.

If desired, dietary fibers can also be incorporated within the functional food composition as described herein. Dietary fiber is the edible part of plants or analogous carbohydrates that are resistant to digestion and absorption in the human small intestine, with complete or partial fermentation in the large intestine. Dietary fiber includes polysaccharides, oligosaccharides, lignin, and associated plant substances. Dietary fibers promote beneficial physiologic effects including laxation, and/or constipation relief, and/or blood cholesterol attenuation, and/or blood glucose attenuation. Dietary fibers also enhance a satiety effect, wherein food portions can become effectively smaller while maintaining a feeling of fullness after a meal.

Other compounds can be used exclusively or in combination with bioflavonoids, carotenes and essential fatty acid compounds of the functional food compositions described herein. Nutraceuticals are generally thought of as food or food product that reportedly provides health and medical benefits, including the prevention and treatment of disease, and can be defined as a product isolated or purified from foods that is generally sold in medicinal forms not usually associated with food. A nutraceutical may have a physiological benefit or provide protection against chronic disease. Such nutraceutical products may range from isolated nutrients, vitamins and minerals, other dietary supplements, and herbal products. With recent developments in cellular-level nutraceutical agents, researchers, and medical practitioners are developing templates for integrating and assessing information from clinical studies on complementary and alternative therapies into responsible medical practice.

Cardiovascular Health Compositions with Bioflavonoids, Carotenes and Essential Fatty Acid Compounds

In general antioxidants contemplated for use for the functional food composition as described herein can include, without limitation carotenes, bioflavonoids (flavonoids, isoflavones, neoflavonals), tocopherol, tocotrienol, lipoic acid, melatonin, superoxide dismutase, coenzyme Q10, alpha lipoic acid, vitamin A, chromium biotin, selenium and ascorbic acid. Carotenes contemplated for use as membrane particles include α-carotene, β-carotene, γ-carotene, δ-carotene, ε-carotene, lycopene, cryptoxanthin, lutein, zeaxathin, apocarotenal astaxanthin, canthaxanthin, lutein/lutein esters, etc. If desired, the amount of carotene in the functional food composition serving as described herein can be up to about 1 milligram (mg), up to about 5 mg, up to about 10 mg, up to about 15 mg, up to about 20 mg, up to about 25 mg, up to about 30 mg, up to about 35 mg, up to about 35 mg, up to about 40 mg, up to about 45 mg, up to about 50 mg, up to about 60 mg, up to about 70 mg, up to about 80 mg, up to about 90 mg, up to about 100 mg. Bioflavonoids used for the functional food composition described herein include esveratrol, quercetin, rutin, catechin, epichatechins, proanthocyanidins, and combinations thereof. Bioflavonoids can be derived from various plants including, for example, Camellia sinensis, Theobroma cacao (cocoa flavanols) and species, varieties, cultivars, thereof. Bioflavonoids can also be derived from or found in various plant extracts including, but not limited to, acai berry extract, raspberry extract, cranberry extract, pomegranate extract, plum extract, cherry extract, rosemary extract, etc. Isoflavones are used as membrane particles, including, but not limited to, genistein, daidzein, biochanin A, and formononetin. If desired, the amount of bioflavonoid in the functional food composition serving as described herein can be up to about up to about 50 mg, up to about 100 mg, up to about 150 mg, up to about 200 mg, up to about 250 mg, up to about 300 mg, up to about 350 mg, up to about 400 mg, up to about 450 mg, up to about 500 mg, up to about 600 mg, up to about 700 mg, up to about 800 mg, up to about 900 mg, up to about 1000 mg.

Essential fatty acids for the functional food composition as described herein can include, without limitation, all-cis-7,10,13-hexadecatrienoic acid (HTA), all-cis-9,12,15-octadecatrienoic acid (ALA), all-cis-6,9,12,15-octadecatetraenoic acid, all-cis-11,14,17-eicosatrienoic acid (ETE), all-cis-8,11,14,17-eicosatetraenoic acid (ETA), all-cis-5,8,11,14,17-eicosapentaenoic acid (EPA), all-cis-6,9,12,15,18-heneicosapentaenoic acid (HPA), all-cis-7,10,13,16,19-docosapentaenoic acid(DPA), all-cis-4,7,10,13,16,19-docosahexaenoic acid (DHA), all-cis-9,12,15,18,21-tetracosapentaenoic acid, all-cis-6,9,12,15,18,21-tetracosahexaenoic acid (nisinic acid). If desired, the amount of essential fatty acids in the functional food composition as described herein can be up to about 3 grams. In particular, essential fatty acid ETA can be provided in the composition at about 50 milligrams (mgs), up to about 100 mgs, up to about 200 mgs, up to about 300 mgs, up to about 400 mgs, up to about 500 mgs, up to about 600 mgs, up to about 700 mgs, up to about 800 mgs, up to about 900 mgs, up to about 1000 mgs, up to about 1200 mgs, up to about 1400 mgs, up to about 1600 mgs, up to about 1800 mgs, up to about 2000 mgs, up to about 2500 mgs, up to about 3000 mgs. If desired, the amount of essential fatty acids in the functional food composition as described herein can be up to about 3 grams. In particular, essential fatty acid DHA can be provided in the composition at about 50 milligrams (mgs), up to about 100 mgs, up to about 200 mgs, up to about 300 mgs, up to about 400 mgs, up to about 500 mgs, up to about 600 mgs, up to about 700 mgs, up to about 800 mgs, up to about 900 mgs, up to about 1000 mgs, up to about 1200 mgs, up to about 1400 mgs, up to about 1600 mgs, up to about 1800 mgs, up to about 2000 mgs, up to about 2500 mgs, up to about 3000 mgs.

Uses of Edible Cardiovascular Health Compositions

Functional food compositions described herein are contemplated to provide a functional benefit to a consumer (“functional food”). Functional foods are formulated to provide an important nutritive food source that contributes to the prevention, management and/or treatment of chronic diseases and/or maintenance of a healthy state, as well as provide a source of bioflavonoids, carotenes and essential fatty acid compounds for cardiovascular health, treatment and maintenance. These benefits can be provided to a consumer when new ingredients or more of an existing ingredient are added to a food so that an added health benefit from the food product is gained.

Supplementing a diet with bioflavonoids, carotenes and essential fatty acids deliverable in the functional foods as described herein is useful for prevention and treatment of a variety of conditions and diseases related developmental disorders, particularly those related to cardiovascular disease, including high blood cholesterol, high blood pressure, arterial plaque development and atherosclerosis, for the mitigation of cardiovascular events including stroke and heart attack, as well as for general health maintenance.

The functional food compositions as described herein increase the effective delivered concentration of such useful bioflavonoids, carotenes and essential fatty acid compounds, and can be formulated for daily dietary supplements, preventative treatment or for clinical applications treating systemic or chronic disease states and disorders.

VI. Overview of the Functional Nutrition Compositions for Joint and Cartilage Health

The disclosure relates to functional food compositions that contain compounds when, upon ingestion, promote joint and cartilage structure health and well-being and maintenance of cartilage structure. The functional food composition contains an edible or potable substance that is encapsulated in an edible membrane, and further contains compounds for joint and cartilage structure health. The functional food compositions described herein provide a convenient and improved way for individuals to consume joint and cartilage structure health compounds in combinations and/or sufficient quantities needed to promote joint and cartilage structure health and well-being related thereto.

Generally, functional food compositions are intended to provide a healthy benefit to a consumer. The benefit can be qualitative, quantitative or subjective and include, for example, health promotion, disease prevention, etc. Some benefits arise from providing an important nutritive food source, vitamins and supplements and/or compounds that generally or specifically contribute to increased health. A higher degree of health assists in the prevention, management and treatment of acute or chronic diseases or disorders. This health improvement benefit can be provided to a consumer when additional ingredients or more of an existing ingredient are added to a food so that the benefit from the food composition is gained. However, delivery systems for some bioavailable compounds, including those important for joint and cartilage health, can reduce the viability of the compounds, resulting in suboptimal quantities of these compounds being made available to the consumer for a particular food composition.

The functional food compositions described herein provide for a significant improvement in the delivery, viability and concentration of compounds useful for joint and cartilage health and maintenance available to a consumer. Within these compositions, the joint and cartilage health compounds can be compartmentalized within the edible or potable substance, the encapsulating membrane, or both. Upon mastication and ingestion, the joint and cartilage health compounds are released from the functional food composition to provide an immediately available nutrition source. The functional food compositions can thus be designed to have one or more joint and cartilage health compounds in an effective combination and concentration desirable for the specific needs of the consumer or patient, for example, elderly, children, and medical patients.

The functional food compositions described herein are designed to provide joint and cartilage health compounds in biologically effective quantities superior to commonly used products. The compositions are particularly well-suited for providing a larger active ingredient load as well as being amenable for controlling the actual portion size for an intended health benefit. Functional food compositions as described herein can provide up to and above several times the suggested minimum dosages in a simple serving, or provide combinations of joint and cartilage health compounds, vitamins and supplements up to and above several times the suggested minimum dosages in a single serving that is both convenient and appetizing. A functional food delivering a combination of vitamins, supplements and joint and cartilage health compounds in a single product can benefit from a resulting synergistic interaction, as has been identified, for example, in patients and consumers suffering joint pain from a variety of causes, including arthritic diseases such as osteoarthritis and rheumatoid arthritis. Apart from joint pain, insufficient maintenance of joint cartilage can result in immobility and joint disfigurement.

Population segments that may find useful a functional food joint and cartilage health composition as described herein include the elderly, athletes, laborers, and patients having undergone reparative surgery, reconstructive surgery and the like. These consumers and patients can become susceptible to arthritis, osteoarthritis, chronic pain, reduction in joint mobility and function, joint effusion, joint erosion, joint inflammation, synovitis, rheumatoid arthritis, psoriatic arthritis, osteoarthrosis, and acute inflammation.

The functional food compositions described herein also enhance the stability, gustatory experience, and delivery of functional biologically active compounds. The functional food compositions contain and protect ingestible/edible substances, such as ice cream, yogurts, etc. and joint and cartilage health compounds, by encapsulating the substance within edible membranes, for which the membranes provide barriers to oxygen, diffusion, etc. The edible membranes of these compositions can be formed from various natural polymeric substances that allow the compositions to be easily transported and consumed by a consumer.

Various supplements, nutraceuticals and products for joint and cartilage health also can be included in the functional food compositions described herein in quantities sufficient to correlate with increased state of health and beneficial treatment of joint and cartilage disorders, in a form that provides a tasty and delicious gustatory experience. The functional food compositions for use in joint and cartilage health and nutrition described herein include, but are not limited to, bioavailable forms of collagen, glucosamine, hyaluronic acid, and chondroitin, other compounds that act as catalysts or supply raw materials for bone and connective tissue synthesis (S-adenosylmethionione (SAM), methylsulfonylmethane (MSM)), vitamins and minerals, anti-oxidants, anti-inflammatory compounds, and essential fatty acids.

Functional nutrition compounds specific for joint and cartilage health have been correlated with disease prevention and general health and wellbeing. For example, glucosamine and chondroitin have been used for the therapeutic treatment of osteoarthritis/arthritis, joint pain mitigation, and preventative health maintenance of joint cartilage. Additionally, compounds including collagen and hyaluronic acid have been used to some effectiveness in the treatment of joint and cartilage disease and disorders, or as a maintenance supplement for joint and cartilage health.

Glucosamine (glucosamine sulfate, glucosamine hydrochloride, and/or N-acetylglucosamine) is a nitrogen containing sugar with important roles in biochemical pathways for the production of glycosolated protein, lipids, and all nitrogen containing sugars. Since glucosamine is a precursor for glycosaminoglycans, a major component of joint cartilage, glucosamine supplements are thought to contribute to the development and maintenance of cartilage structure, and thus the alleviation of arthritis.

Chondroitin sulfate likely results in anti-inflammatory activity through the promotion or synthesis of beneficial metabolites (proteoglycans and hyaluronic acid), and the attenuation of synthesis of harmful catabolites that further inhibit the synthesis of proteolytic enzymes, nitric oxide, and other substances that contribute to damage of cartilage matrix and cause death of articular chondrocytes. Therefore, treatment of arthritis and other cartilage and joint conditions with chondroitin sulfate is based on the belief that osteoarthritis is associated with a local deficiency or degradation of natural substances, including internal chondroitin sulfate.

Generally, glucosamine and/or chondroitin and/or metabolic derivatives and/or complexes thereof, can be incorporated into the membrane and/or edible or potable substance. Some servings of the functional food composition herein provide up to about up to about 2000 mgs total of one or more of glucosamine, chondroitin, derivatives thereof, and/or complexes thereof.

Collagen is a group of naturally occurring proteins found in the skin and connective tissues of animals, making up about one third of the whole-body protein content. Collagen occurs in many places throughout the body and comprises the main component of connective tissue. Many types of collagen are known, the most common being collagen I (skin, tendon, vascular ligature, organs, the main component of the organic part of bone), collagen II (main component of cartilage), collagen III (main component of reticulate fibers and commonly found alongside type I), collagen IV (forming the bases of cell basement membrane), and collagen V (cell surfaces, hair and placenta). Collagen-related diseases most commonly arise from genetic defects or nutritional deficiencies that affect the biosynthesis, assembly, postranslational modification, secretion, or other processes involved in normal collagen production. Hydrolyzed and non-hydrolyzed type II collagen, often in combination with other joint and cartilage health compounds, can be used for joint pain relief and treatment of arthritis and other joint conditions and diseases.

Hyaluronic acid is a polymer of disaccharides composed of glucuronic acid and N-acetylglucosamine, and is a major component of synovial fluid. Hyaluronic acid functions to increase the viscosity of the fluid and increase effectiveness of lubrication properties. Hyaluronan is also an important component of articular cartilage where it is present as a coat around each chondrocyte cell. Since hyaluronic acid is a major component of joint cartilage, hyaluronic acid supplements are thought to contribute to the development and maintenance of cartilage structure, and thus the alleviation of arthritis and other joint and cartilage disorders.

In general, a reduction in pain and discomfort, improvement in quality of life and ability to perform activities of daily living, and measurable differences in concentration of metabolites or cartilage density are all indicators of the health benefits when consuming the functional food compositions for joint and cartilage health described herein.

Joint and Cartilage Health Compositions

Compounds in concentration and/or type contained within the functional food compositions included herein may be used in addition to natural levels of joint and cartilage health compounds in the food source.

Generally, chondroitin and bioavailable and bioactive metabolic derivatives and/or complexes can be included in membranes encapsulating an edible or potable substance and/or within the edible or potable substance. Chondroitin sulfate, itself metabolized from a glucosamine precursor, is a nitrogen containing sugar composed of a chain of alternating N-acetylgalactosamine and glucuronic acids, each potentially sulfated at any of 3 atom positions to yield a number of chondroitin sulfate fractions (chondroitin-4-sulfate, chondroitin-6-sulfate, chondroitin-4,6-sulfate, chondroitin-2,6-sulfate). Other chondroitin forms for use in the functional food composition described herein include chondroitin chloride, chondroitin bromide, chondroitin sulfate, and chondroitin iodide. If desired, the amount of chondroitin provided in a joint and cartilage health functional food composition can be sufficient for a recommended daily intake, for example, 200 mgs total, up to about 400 mgs total, up to about 600 mgs total, up to about 800 mgs total, up to about 1000 mgs total, up to about 1200 mgs total, up to about 1400 mgs total, up to about 1600 mgs total, up to about 1800 mgs total, up to about 2000 mgs.

Generally, glucosamine is a nitrogen containing sugar with important roles in biochemical pathways for the production of glycosolated protein, lipids, and all nitrogen containing sugars. Bioavailable and bioactive forms of glucosamine include glucosamine sulfate, glucosamine hydrochloride, and/or N-acetylglucosamine, glucosamine hydroiodide, glucosamine pyruvate, glucosamine phosphate, β-glucosamine, and α-glucosamine. If desired, the amount of glucosamine provided in a joint and cartilage health functional food composition can be sufficient for a recommended daily intake, for example, 200 mgs total, up to about 400 mgs total, up to about 600 mgs total, up to about 800 mgs total, up to about 1000 mgs total, up to about 1200 mgs total, up to about 1400 mgs total, up to about 1600 mgs total, up to about 1800 mgs total, up to about 2000 mgs.

Generally, collagen compounds comprising type II and/or hydrolyzed type II collagen have been shown to be most effective for treatment of joint and cartilage health. If desired, the amount of collagen provided in a joint and cartilage health functional food composition can be sufficient for a recommended daily intake, for example, up to 1000 mgs total, up to about 2000 mgs total, up to about 3000 mgs total, up to about 4000 mgs total, up to about 5000 mgs total, up to about 6000 mgs total, up to about 7000 mgs total, up to about 8000 mgs total, up to about 9000 mgs total, up to about 10,000 mgs.

Generally, hyaluronic compounds and/or salts, have been shown to be effective for treating joint pain. Bioavailable forms of hyaluronic acid include, for example, sodium hyaluronate, potassium hyaluronate, magnesium hyaluronate and calcium hyaluronate. If desired, the amount of hyaluronic acid provided in a joint and cartilage health functional food composition can be sufficient for a recommended daily intake, for example, up to about 10 mg, up to about 50 mgs, up to about 100 mgs, up to about 500 mgs, up to about 1000 mgs, up to about 1500 mgs, up to about 2000 mgs, up to about 2500 mgs, up to about 3000 mgs.

Generally, other compounds and/or bioavailable metabolic derivatives can include vitamins (for example, vitamin A, vitamin B, vitamin C, vitamin D, vitamin E, vitamin K, etc), minerals (for example calcium, iron, copper, zinc, magnesium, manganese, phosphorus, potassium, etc.), essential fatty acids (for example, omega-3-fatty acid, omega-6-fatty acid, etc.), essential amino acids (L-histidine, L-isoleucine, L-leucine, L-lysine, L-methionine, L-phenylalanine, L-valine and L-threonine), antioxidant agents and anti-inflammatory agents.

Uses of Edible Compositions for Joint and Cartilage Health and Maintenance

Edible transport compositions described herein are contemplated to provide a functional benefit to a consumer (“functional food”). Functional foods are formulated to provide an important nutritive food source that contributes to the prevention, management and/or treatment of chronic diseases. This benefit can be provided to a consumer when new ingredients or more of an existing ingredient are added to a food so that an added health benefit from the food product is gained. Joint and cartilage health compounds such as glucosamine, chondroitin, collagen, hyaluronic acid or supplements including, without limitation, vitamins, minerals, amino acids, antioxidants, anti-inflammatory agents, and essential fatty acids are useful for a variety of conditions and diseases as well as general health maintenance. The effectiveness of joint and cartilage health treatment and maintenance is enhanced through the use of an efficient delivery system. The functional food compositions as described herein increase the effective delivered concentration of such useful joint and cartilage health compositions, and can be formulated for daily dietary supplements, preventative treatment or for clinical applications treating systemic disease states.

Supplementing daily dietary intake with joint and cartilage health compounds as a functional food composition contributes to maintenance of joints and cartilage, an important health concern that increases with physical stresses and age. In older adults, the breakdown of cartilage exceeds the rate of replacement. Various diseases and conditions can present in a consumer or patient from insufficient cartilage maintenance, including osteoarthritis, chronic pain, reduction in joint mobility and function, joint effusion, joint erosion, joint inflammation, synovitis, rheumatoid arthritis, psoriatic arthritis, osteoarthrosis, and acute inflammation.

Maintenance treatment in a daily diet with functional food compositions directed to general joint and cartilage health (up to about 2000 milligrams chondroitin, up to about 2000 milligrams of glucosamine, up to about 200 milligrams hyaluronic acid, and up to about 3000 milligrams of collagen) can be useful for the prevention of cartilage breakdown and loss due to aging, the prevention of disease states, for example for example arthritis/degenerative joint disease, mitigating chronic joint pain, and contributing to general health and wellness of the cartilage and joints.

VII. Overview of the Functional Nutrition Compositions Containing Multivitamins

The disclosure relates to functional food compositions that contain compounds when, upon ingestion, provide nutritional supplements and an increase in general health and well-being. The functional food composition contains an edible or potable substance that is encapsulated in an edible membrane, and further contains multivitamin compounds supplementing a normal diet or for use with dietary imbalances. The functional food compositions described herein provide a convenient and improved way for individuals to consume multivitamin compounds in combinations and/or sufficient quantities needed to supplement normal diet, treat dietary imbalances, and increase general health and well-being related thereto.

Generally, functional food compositions are intended to provide a healthy benefit to a consumer. The benefit can be qualitative, quantitative or subjective and include, for example, health promotion, disease prevention, etc. Some benefits arise from providing an important nutritive food source, vitamins and supplements and/or compounds that generally or specifically contribute to increased health. A higher degree of health assists in the prevention, management and treatment of acute or chronic diseases or disorders. This health improvement benefit can be provided to a consumer when additional ingredients or more of an existing ingredient are added to a food so that the benefit from the food composition is gained. However, delivery systems for some bioavailable compounds, including those important for supplementing a diet, can reduce the viability of the compounds, resulting in suboptimal quantities of these compounds being made available to the consumer for a particular food composition. Often, the delivery system for a nutritional supplement is in a form insufficient to promote compliance in a particular consumer group (e.g., children and young adults), such as being in a pill form, powder form with little gustatory appeal, or poor tasting liquids.

The functional food compositions described herein provide for a significant improvement in the delivery, viability and concentration of multivitamin compounds useful to a consumer. Within these compositions, the multivitamin compounds can be compartmentalized within the edible or potable substance, the encapsulating membrane, or both. Upon mastication and ingestion, the multivitamin compounds are released from the functional food composition to provide an immediately available nutrition and edible source. The functional food compositions can thus be designed to have one or more multivitamin compounds in an effective combination and concentration desirable for the specific needs of the consumer or patient, for example, the elderly, adult and children consumers, and medical patients.

The functional food compositions described herein are designed to provide multivitamin compounds in biologically effective quantities superior to commonly used products. The compositions are particularly well-suited for providing a larger active ingredient load as well as being amenable for controlling the actual portion size for an intended health benefit. Functional food compositions as described herein can provide up to and above several times the suggested minimum dosages in a sample serving, or provide combinations of multivitamin compounds, nutraceuticals and supplements up to and above several times the suggested minimum dosages in a single serving that is both convenient and appetizing. A functional food delivering a combination of multivitamins and supplements in a single serving composition can result in the cessation or mitigation of a multitude of health related conditions and diseases, supplement missing or low concentrations of vitamins and minerals in a dietary imbalance, as well as promote compliance in the daily consumption of multivitamin supplements. In addition, a general state of health and wellness can result from a healthy diet supplemented with multivitamins.

Population segments that may find useful a multivitamin composition as described herein include the elderly, young adults, youths and hospital patients. Consumers and patients can become noncompliant for daily intake of multivitamin supplements depending on the delivery form of the multivitamin composition. For example, some multivitamins are in pill form with a flavor, texture and overall size that often results in reluctance by children to consume. A tablet or pill structure often has negative associations made by children, who confuse a multivitamin pill with a pharmacological pill. For those tablets with added sweeteners that are designed to be playful or mimic confections, children can confuse those multivitamin pills with candy and be in danger of over-consumption. For multivitamins designed for adult consumption, children may mistake those multivitamins compositions with compositions formulated for children. In these instances, children are at risk of toxic ingestion, for example iron. Therefore, a functional food as described herein that delivers a daily allowance of multivitamins can overcome non-compliance and potential for confusion with a confection, adult formulations, and/or over-consumption.

The functional food compositions described herein also enhance the stability, gustatory experience, and delivery of functional biologically active compounds. The functional food compositions contain and protect ingestible/edible substances, such as ice cream, yogurts, etc. and multivitamin compounds, by encapsulating the substance within edible or biodegradable membranes, for which the membranes provide barriers to oxygen, diffusion, etc. The edible membranes of these compositions can be formed from various natural polymeric substances that allow the compositions to be easily transported and consumed by a consumer.

Various vitamins, supplements, and nutraceuticals can be included in the functional food compositions described herein in quantities sufficient to correlate with recommended daily allowances and beneficial supplementation to normal dietary intake, in a form that provides a tasty and delicious gustatory experience. The functional food compositions for use in multivitamin supplementation and general nutrition described herein include, but are not limited to, bioavailable forms of vitamins, derivatives, salts thereof, and bioactive metabolic intermediates.

Generally, multivitamins as used herein can be considered as vitamins and/or minerals taken in addition to naturally obtained vitamins/minerals in food. Multivitamins are taken 1) to enhance the physical well-being or state of health of the end user, 2) as a health related supplement, 3) as supplements required for enhancing deficient vitamin/mineral states in the end user, and/or 4) as supplements to mitigate low concentrations or missing vitamins and minerals due to dietary imbalances (e.g. “finicky” eaters, vegetarians, highly active people as well as those on specialized diets, sufferers of chronic illnesses such as diabetes, persons having genetic diseases, etc.). Dietary supplements can also add to a higher quality or perceived quality of the health state of the end user.

If desired, vitamins and/or minerals that can be toxic to specific population segments can be eliminated from the functional food formulations. For example, iron toxicity is possible for young adult and child consumers. Therefore, in these formulations it may be desirable to provide a functional food formulation with iron fortification.

Other compounds can be used exclusively or in combination with multivitamins functional food compositions described herein. Nutraceuticals are generally thought of as food or food product that reportedly provides health and medical benefits, including the prevention and treatment of disease, and can be defined as a product isolated or purified from foods that is generally sold in medicinal forms not usually associated with food. A nutraceutical may have a physiological benefit or provide protection against chronic disease. Such nutraceutical products may range from isolated nutrients, dietary supplements, herbal products, and fiber. With recent developments in cellular-level nutraceutical agents, researchers, and medical practitioners are developing templates for integrating and assessing information from clinical studies on complementary and alternative therapies into responsible medical practice.

Multivitamin Compositions

Compounds in concentration and/or type contained within the functional food compositions included herein may be used in addition to natural levels of multivitamin compounds in the food source.

Generally, multivitamins and bioavailable and bioactive metabolic derivatives and/or complexes can be included in membranes encapsulating an edible or potable substance and/or within the edible or potable substance. Concentrated, isolated and combinations of compounds useful as multivitamins are commercially available. Generally, vitamins, minerals and other supplements that can be used in the multivitamin functional food can be, without limitation, ascorbic acid (vitamin C), vitamin B₁₂, biotin, fat soluble vitamins, folic acid, HCA (hydroxycitric acid), inositol, pyruvate, mineral ascorbates, mixed tocopherols, niacin (vitamin B₃), orotic acid, PABA (para-aminobenzoic acid), pantothenates, pantothenic acid (vitamin B₅), pyridoxine hydrochloride (vitamin B₆), riboflavin (Vitamin B₂), synthetic vitamins, thiamine (vitamin B₁, tocotrienols, vitamin A, vitamin D₃, vitamin E, vitamin F (linoleic acid (LA) and alpha-linoleic acid (LNA)), vitamin K₁, vitamin K₂, vitamin oils, vitamin premixes, vitamin-mineral premixes, water soluble vitamins, arsenic, boron, calcium, chloride, chromium, cobalt, copper, fluorine, iodine, iron, magnesium, manganese, molybdenum, nickel, phosphorous, potassium, selenium, silicon, sodium, strontium, sulfur, vanadium, zinc, combinations thereof, etc. If desired, the amount of vitamins, minerals and/or other supplements provided in a multivitamin functional food composition sufficient for an effective daily intake can be, for example, up to 20% of recommended daily allowance (RDA), up to 30% RDA, up to 40% RDA, up to 50% RDA, up to 60% RDA, up to 70% RDA, up to 80% RDA, up to 90% RDA, up to 100% RDA, up to 500% RDA, up to 1000% RDA, up to 2000% RDA, up to 3000% RDA, up to 4000% RDA, up to 5000% RDA.

If desired, enzymes and amino acids can be included in the functional food as described herein, and include, but are not limited to, alpha galactosidase, amylase, bromelain, cellulase, papain, peptidase, protease, proteolytic enzymes, superoxide dismutase, trypsin, betaine, casein, glutamic Acid, L-alanine, L-arginine, L-cysteine, L-glutamine, L-glycine, L-isoleucine, L-leucine, L-methionine, L-ornithine, L-phenylalanine, L-proline, L-taurine, L-threonine, L-tryptophan, L-tyrosine, L-valine, N-acetly-L-cysteine, protein soluble soy, soy protein isolates, whey protein isolates, combinations thereof, etc.

If desired, antioxidants can be included in the functional food as described herein, and include, but are not limited to, carotenoids, flavonoids, isoflavones, tocopherol, tocotrienol, lipoic acid, melatonin, superoxide dismutase, coenzyme Q10, alpha lipoic acid, vitamin A, chromium biotin, selenium, ascorbic acid combinations thereof, etc. Carotenoids include include alpha-carotene, beta-carotene, cryptoxanthin, lycopene, lutein, zeaxathin, apocarotenal astaxanthin, canthaxanthin, lutein/lutein esters, combinations thereof, etc. Flavonoids include used as membrane particles include esveratrol, quercetin, rutin, catechin, proanthocyanidins, acai berry extract, raspberry extract, cranberry extract, pomegranate extract, plum extract, cherry extract, rosemary extract, combinations thereof, etc. Isoflavones include genistein, daidzein, biochanin A, and formononetin, combinations thereof, etc.

Uses of Edible Multivitamin Compositions

Functional food compositions described herein are contemplated to provide a functional benefit to a consumer (“functional food”). Functional foods are formulated to provide an important nutritive food source that contributes to the prevention, management and/or treatment of chronic diseases and/or maintenance of a healthy state, as well as provide a vitamin and mineral supplementing resource in applications where dietary imbalance is evident. This benefit can be provided to a consumer when new ingredients or more of an existing ingredient are added to a food so that an added health benefit from the food product is gained. Multivitamin compounds are useful for prevention and treatment of a variety of conditions and diseases related to deficient vitamin and/or mineral intake, as well as general health maintenance. The functional food compositions as described herein increase the effective delivered concentration of such useful multivitamin compositions, and can be formulated for daily dietary supplements, preventative treatment or for clinical applications treating systemic or chronic disease states and disorders.

VIII. Overview of the Functional Nutrition Compositions Containing Essential Fatty Acids

The disclosure relates to functional food compositions that contain compounds when, upon ingestion, provide nutritional supplements and an increase in general health and well-being. The functional food composition contains an edible or potable substance that is encapsulated in an edible membrane, and further contains essential fatty acids and dietary fiber compounds supplementing a normal diet. The functional food compositions described herein provide a convenient and improved way for individuals to consume essential fatty acid compounds and optionally dietary fiber in combinations and/or sufficient quantities needed to supplement normal diet, promote normal growth and development, treat disease conditions, and increase general health and well-being related thereto.

Generally, functional food compositions are intended to provide a healthy benefit to a consumer. The benefit can be qualitative, quantitative or subjective and include, for example, health promotion, disease prevention, etc. Some benefits arise from providing an important nutritive food source, vitamins and supplements and/or compounds such as essential fatty acids and dietary fiber that generally or specifically contribute to increased health. A higher degree of health assists in the prevention, management and treatment of acute or chronic diseases or disorders. This health improvement benefit can be provided to a consumer when additional ingredients or more of an existing ingredient are added to a food so that the benefit from the food composition is gained. However, delivery systems for some bioavailable compounds, including those important for supplementing a diet, can reduce the viability of the compounds, resulting in suboptimal quantities of these compounds being made available to the consumer for a particular food composition. Often, the delivery system for a nutritional supplement is in a form insufficient to promote compliance in a particular consumer group (e.g., children and young adults), such as being in a large pill form, powder form with little gustatory appeal, or poor tasting liquids.

The functional food compositions described herein provide for a significant improvement in the delivery, viability and concentration of essential fatty acid compounds and dietary fiber useful to a consumer. Within these compositions, the essential fatty acid compounds and dietary fiber can be compartmentalized within the edible or potable substance, the encapsulating membrane, or both. Upon mastication and ingestion, the essential fatty acid compounds and optionally dietary fiber are released from the functional food composition to provide an immediately available nutrition and edible source. The functional food compositions can thus be designed to have one or more essential fatty acid compounds and dietary fiber in an effective combination and concentration desirable for the specific needs of the consumer or patient, for example, the elderly, adult and children consumers, and medical patients.

The functional food compositions described herein are designed to provide essential fatty acid compounds and dietary fiber in biologically effective quantities superior to commonly used products. The compositions are particularly well-suited for providing a larger active ingredient load as well as being amenable for controlling the actual portion size for an intended health benefit. Functional food compositions as described herein can provide up to and above several times the suggested minimum dosages in a sample serving, or provide combinations of essential fatty acid compounds and dietary fiber, nutraceuticals and supplements up to and above several times the suggested minimum dosages in a single serving that is both convenient and appetizing. A functional food delivering a combination of essential fatty acid compounds, dietary fiber and supplements in a single serving composition can result in the cessation or mitigation of a multitude of health related conditions and diseases, supplement dietary imbalance, promote normal development of organ systems such as nerves, brain and eyes, treat disease (e.g., coronary disease) and related disorders (e.g., high cholesterol, high blood pressure, etc.) and promote compliance in the daily consumption of these and other health promoting compounds. In addition, a general state of health and wellness can result from a healthy diet supplemented with these and other health promoting compounds.

Population segments that may find useful an essential fatty acid and dietary fiber functional food composition as described herein include the elderly, adults, young adults, youths and hospital patients. Consumers and patients can become noncompliant for daily intake of essential fatty acids and dietary fiber compounds depending on the delivery form of the composition. For example, some essential fatty acids are in pill form with a flavor, texture and overall size that often results in reluctance by children to consume. A tablet or pill structure often has negative associations made by children, who confuse a supplement pills with a pharmacological pill. Therefore, a functional food as described herein that delivers a daily allowance of essential fatty acids and dietary fiber can overcome non-compliance.

The functional food compositions described herein also enhance the stability, gustatory experience, and delivery of functional biologically active compounds. The functional food compositions contain and protect ingestible/edible substances, such as ice cream, yogurts, etc. and essential fatty acid compounds and dietary fiber, by encapsulating the substance within edible or biodegradable membranes, for which the membranes provide barriers to oxygen and oxygen degradation, diffusion, etc. The double bond(s) found within essential fatty acid compounds are particularly susceptible to oxidative degradation, and thus it is desirable to ensure protection against oxygen. The edible membranes of these compositions can be formed from various natural polymeric substances that allow the compositions to be easily transported and consumed by a consumer.

Various essential fatty acid compounds, dietary fiber, supplements, and nutraceuticals can be included in the functional food compositions described herein in quantities sufficient to correlate with recommended daily allowances, promotion of organ system development, and beneficial supplementation to normal dietary intake, in a form that provides a tasty and delicious gustatory experience. The functional food compositions for use with essential fatty acid and dietary fiber compounds described herein include, but are not limited to, bioavailable forms of essential fatty acids and dietary fibers, bioactive derivatives thereof, salts thereof, and bioactive metabolic intermediates.

Generally, essential fatty acids and dietary fiber as used herein can be considered supplementing naturally obtained essential fatty acids and dietary fiber in food. Essential fatty acids are consumed as part of a healthy diet in order to maintain and promote the development of certain organ systems including eyes, nerves, brain, etc. (DHA, in particular). The fatty acid compounds are also used for the treatment and/or prevention of cardiovascular disease and related disorders and events, such as high blood pressure, high cholesterol, the onset of arthrosclerosis, heart attack and stroke. Essential fatty acids can also add to a higher quality or perceived quality of the health state of the end user, as some correlations have been identified with decrease in cancer risk, effectiveness against arthritis, dementia and depression.

If desired, dietary fibers can also be incorporated within the functional food composition as described herein. Dietary fiber is the edible parts of plants or analogous carbohydrates that are resistant to digestion and absorption in the human small intestine, with complete or partial fermentation in the large intestine. Dietary fiber includes polysaccharides, oligosaccharides, lignin, and associated plant substances. Dietary fibers promote beneficial physiologic effects including laxation, and/or constipation relief, and/or blood cholesterol attenuation, and/or blood glucose attenuation. Dietary fibers also enhance a satiety effect, wherein food portions can become effectively smaller while maintaining a feeling of fullness after a meal.

Other compounds can be used exclusively or in combination with essential fatty acids and dietary fiber functional food compositions described herein. Nutraceuticals are generally thought of as food or food product that reportedly provides health and medical benefits, including the prevention and treatment of disease, and can be defined as a product isolated or purified from foods that is generally sold in medicinal forms not usually associated with food. A nutraceutical may have a physiological benefit or provide protection against chronic disease. Such nutraceutical products may range from isolated nutrients, vitamins and minerals, other dietary supplements, and herbal products. With recent developments in cellular-level nutraceutical agents, researchers, and medical practitioners are developing templates for integrating and assessing information from clinical studies on complementary and alternative therapies into responsible medical practice.

Essential Fatty Acid and Dietary Fiber Compositions

Essential fatty acids for the functional food composition as described herein can include, without limitation, all-cis-7,10,13-hexadecatrienoic acid (HTA), all-cis-9,12,15-octadecatrienoic acid (ALA), all-cis-6,9,12,15-octadecatetraenoic acid, all-cis-11,14,17-eicosatrienoic acid (ETE), all-cis-8,11,14,17-eicosatetraenoic acid (ETA), all-cis-5,8,11,14,17-eicosapentaenoic acid (EPA), all-cis-6,9,12,15,18-heneicosapentaenoic acid (HPA), all-cis-7,10,13,16,19-docosapentaenoic acid(DPA), all-cis-4,7,10,13,16,19-docosahexaenoic acid (DHA), all-cis-9,12,15,18,21-tetracosapentaenoic acid, all-cis-6,9,12,15,18,21-tetracosahexaenoic acid (nisinic acid). If desired, the amount of essential fatty acids in the functional food composition as described herein can be up to about 3 grams. In particular, essential fatty acid ETA can be provided in the composition at about 50 milligrams (mgs), up to about 100 mgs, up to about 200 mgs, up to about 300 mgs, up to about 400 mgs, up to about 500 mgs, up to about 600 mgs, up to about 700 mgs, up to about 800 mgs, up to about 900 mgs, up to about 1000 mgs, up to about 1200 mgs, up to about 1400 mgs, up to about 1600 mgs, up to about 1800 mgs, up to about 2000 mgs, up to about 2500 mgs, up to about 3000 mgs. If desired, the amount of essential fatty acids in the functional food composition as described herein can be up to about 3 grams. In particular, essential fatty acid DHA can be provided in the composition at about 50 milligrams (mgs), up to about 100 mgs, up to about 200 mgs, up to about 300 mgs, up to about 400 mgs, up to about 500 mgs, up to about 600 mgs, up to about 700 mgs, up to about 800 mgs, up to about 900 mgs, up to about 1000 mgs, up to about 1200 mgs, up to about 1400 mgs, up to about 1600 mgs, up to about 1800 mgs, up to about 2000 mgs, up to about 2500 mgs, up to about 3000 mgs.

Dietary fibers for the functional food composition as described herein can include, both soluble and insoluble dietary fibers, including, but not limited to, galacto-oligosaccharides, inulin and oligofructose (fructo-oligosaccharide), isomalto-oligosaccharides, lactulose, lactosucrose, transgalacto-oligosaccharides, soybean oligosaccharides, tagatose, xylo-oligosaccharides, and combinations thereof. The dietary fiber can be an oligosaccharide fructan including, but not limited to, fructo-oligosaccharide and derivatives thereof of inulin (poly-D-fructose, itself derived from various plant products including bananas, onions, chicory root, garlic, asparagus, barley, wheat, jicama, leeks, etc.) or from transfructosylation action of a β-fructosidase as used, for example, by Aspergillus sp. metabolically active on sucrose. Dietary fibers can be derived from plants and plant products including, but not limited to, artichoke, dandelion greens, bran, flour, legumes, oats, citrus fruits, apples, and root vegetables, cane sugar, etc. If desired, the amount of total dietary fiber per function food serving can be less than about 1 gram, less than about 2.5 grams, less than about 5 grams, less than about 7.5 grams, less than about 10 grams, less than about 12.5 grams, less than about 15 grams, less than about 17.5 grams, less than about 20 grams.

Uses of Edible Essential Fatty Acid and Dietary Fiber Compositions

Functional food compositions described herein are contemplated to provide a functional benefit to a consumer (“functional food”). Functional foods are formulated to provide an important nutritive food source that contributes to the prevention, management and/or treatment of chronic diseases and/or maintenance of a healthy state, as well as provide a source of essential fatty acids to promote eye, brain and nerve development, and dietary fiber to gastrointestinal and cardiac health. These benefits can be provided to a consumer when new ingredients or more of an existing ingredient are added to a food so that an added health benefit from the food product is gained.

Supplementing a diet with essential fatty acids deliverable in the functional foods as described herein is useful for prevention and treatment of a variety of conditions and diseases related developmental disorders (particularly those related to eyes, brain and the nervous system), immunity (arthritis), cardiovascular disease (high blood cholesterol, high blood pressure, plaque development), and psychological disorders (depression and dementia), as well as for general health maintenance.

Supplementing a diet with essential fatty acids deliverable in the functional foods as described herein is useful for prevention and treatment of a variety of conditions and diseases. Particular applications include a scheduled dietary fiber intake for prevention of constipation, improvement of gastrointestinal health, a diet aid for use in weight management, reduction in risk of coronary diseases, colon cancer and diabetes.

The functional food compositions as described herein increase the effective delivered concentration of such useful essential fatty acids and dietary fiber compositions, and can be formulated for daily dietary supplements, preventative treatment or for clinical applications treating systemic or chronic disease states and disorders.

IX. Overview of the Branched Chain Amino Acid Functional Food Compositions

The disclosure relates to functional food compositions that contain compounds when, upon ingestion, can increase the general health well-being of the consumer. The functional food composition described herein contains an edible or potable substance that is encapsulated in an edible membrane, and further contains a branched chain amino acid (“BCAA”), supplementing the essential amino acid concentration of a normal diet. The functional food compositions described herein provide a convenient and improved way for individuals to consume BCAAs in combinations and/or sufficient quantities needed to supplement normal diet to treat unhealthy bodily conditions, disease and injury, and increase general health and well-being related thereto.

Generally, functional food compositions are intended to provide a healthy benefit to a consumer. The benefit can be qualitative, quantitative or subjective and include, for example, maintenance and promotion of good health, disease prevention, etc. Some benefits arise from providing an important nutritive food source, vitamins and supplements and/or compounds such as BCAAs that generally or specifically contribute to increased health and wellness. A higher degree of health assists in the prevention, management and treatment of acute or chronic diseases, systemic disorders, trauma and surgery. This health and wellness improvement or benefit can be provided to a consumer when additional ingredients or more of an existing ingredient are added to a food so that the benefit from the food composition is gained. However, delivery systems for some bioavailable compounds, including those important for supplementing a diet, can reduce the viability of the compounds, resulting in suboptimal quantities of these compounds being made available to the consumer for a particular food composition. Often, the delivery system for a nutritional supplement is in a form insufficient to promote compliance in a particular consumer group (e.g., elderly, infirm, children and young adults), because the delivery system is in a large pill form, powder form with little gustatory appeal, or poor tasting liquids.

The functional food compositions described herein provide for a significant improvement in the delivery, viability and concentration of BCAA containing compounds useful to a consumer. Within these compositions, the BCAAs can be compartmentalized within the edible or potable substance, the encapsulating membrane, or both. Upon mastication and ingestion, the BCAAs are released from the functional food composition to provide an immediately available nutrition and edible antioxidant source. The functional food compositions can thus be designed to have one or more BCAAs in an effective combination and concentration desirable for the specific needs of the consumer or patient, for example, the elderly, adult and children consumers, and medical patients.

The functional food compositions described herein are designed to provide BCAAs in biologically effective quantities for promoting general health and well-being and muscle mass retention or development in concentrations superior to commonly used products. The compositions are particularly well-suited for providing a larger active ingredient load as well as being amenable for controlling the actual portion size for an intended health benefit. Functional food compositions as described herein can provide up to and above several times the suggested minimum dosages in a sample serving, or provide desired combinations of BCAAs, nutraceuticals and supplements up to and above several times the suggested minimum dosages in a single serving that is both convenient and appetizing. A functional food delivering BCAAs in a single serving composition can result in the cessation or mitigation of a multitude of conditions and diseases resulting in loss of muscle mass. The functional food described herein can also supplement dietary imbalance and promote user compliance in the daily consumption of these and other health promoting compounds for those desiring to supplement muscle maintenance or development and growth of muscle mass. In addition, a general maintenance state of health and wellness can result from a healthy diet supplemented with these and other functional foods as described herein.

Population segments that may find useful BCAAs as described herein include the elderly, adults, hospital patients, athletes, dieters, and those with a variety of conditions related to trauma, nursing home and hospital care, etc. However, these consumers can become noncompliant for daily intake of health compounds depending on the delivery form of the composition. For example, some health compounds are in pill form with a flavor, texture and overall size (e.g., omega-3 and omega-6 fatty acids, multivitamin supplements, etc.) that often result in reluctance to consume on a scheduled or daily basis. Therefore, a functional food as described herein that delivers a daily concentration of BCAAs can overcome non-compliance.

The functional food compositions described herein also enhance the stability, gustatory experience, and delivery of functional biologically active compounds. The functional food compositions contain and protect ingestible/edible substances, such as ice cream, yogurts, etc., in addition to the BCAAs, by encapsulating the substance within edible or biodegradable membranes, for which the membranes provide barriers to oxygen and oxygen degradation, diffusion, etc. The edible membranes of these compositions can be formed from various natural polymeric substances that allow the compositions to be easily transported and consumed by a consumer.

The BCAAs are essential amino acids in humans needed for normal cellular function including regulation of protein synthesis and degradation. BCAAs, especially leucine, stimulate protein synthesis, inhibit proteolysis (in cell culture models and in animals), and promote glutamine synthesis. Oxidation of BCAAs is controlled by leucine transamination metabolites and is affected by physiological or pathological conditions such as starvation, sepsis, cancer, etc., that result in catabolic states depleted in alanine and glutamine. Supplementing diets with BCAAs during these catabolic states improves synthesis of glutamine to, for example, maintain vital organ function. BCAA increases glutamine synthesis and glutamine release from muscle and elevates glutamine concentration in blood plasma. Some studies indicate that glutamine supplementation improves recovery from infection after various insults such as burns, radiation injury, severe surgical stress, sepsis, and cancer. Additionally, weight loss or wasting and malnutrition often show in patients with prolonged catabolic stress, including, for example, post-operative and trauma care patients. However, maintenance of adequate nutrition can improve their state of health.

Catabolic states can be characterized in part by increased cellular energy consumption, and increased glutamine utilization. Increased BCAA oxidation in skeletal muscle is one of the metabolic responses that compensates for the increased energy expenditure and glutamine consumption. During prolonged, severe stress, increased glutamine utilization can exceed the body's ability to synthesize glutamine, and extensive muscle glutamine depletion may result. During short term stress, intracellular muscle protein and glutamine concentrations decrease and intracellular muscle BCAA concentrations increase. The newly synthesized and mobilized glutamine can help maintain the circulating plasma concentration and increased need for glutamine. Studies have indicated that BCAA supplementation decreases muscle protein catabolism and amino acid efflux, and suggested that the muscle can depend in part on the BCAA supplements to satisfy its metabolic requirements. Therefore, BCAAs as used herein in a functional food composition can be any one of or a combination of L-leucine, L-isoleucine, and L-valine amino acids. Studies have shown that L-leucine is most effective for use as a supplement. Thus it may be desired that L-leucine is supplied preferentially, and if in combination with L-isoleucine and/or L-valine, L-leucine is supplied in a higher proportion to L-isoleucine and/or L-valine.

Generally, glutamine can be used in the BCAA functional food as described herein as an additional supplement, as it has been shown to be useful in treatment of injuries, trauma, burns, and treatment-related side effects of cancer, as well as in wound healing for postoperative patients.

Generally, multivitamins as used herein can be considered as vitamins and/or minerals taken in addition to naturally obtained vitamins/minerals in food. Multivitamins are taken 1) to enhance the physical well-being or state of health of the end user, 2) as a health related supplement, 3) as supplements required for enhancing deficient vitamin/mineral states in the end user, and/or 4) as supplements to mitigate low concentrations or missing vitamins and minerals due to dietary imbalances, disease states, various physiological conditions, various physical conditions, etc. Dietary supplements also may add to a higher quality or perceived quality of the health state of the end user.

If desired, vitamins and/or minerals that can be toxic to specific population segments can be eliminated from the functional food formulations. For example, iron toxicity is possible for young adult and child consumers. Therefore, in these formulations it may be desirable to provide a functional food formulation without iron fortification.

Nutraceuticals are generally thought of as food or food product that reportedly provides health and medical benefits, including the prevention and treatment of disease, and can be defined as a product isolated or purified from foods that is generally sold in medicinal forms not usually associated with food. A nutraceutical may have a physiological benefit or provide protection against chronic disease. Such nutraceutical products may range from isolated nutrients, vitamins and minerals, other dietary supplements, and herbal products. With recent developments in cellular-level nutraceutical agents, researchers, and medical practitioners are developing templates for integrating and assessing information from clinical studies on complementary and alternative therapies into responsible medical practice.

BCAA Functional Food Compositions

Generally, BCAA compositions and isolated compounds thereof, bioavailable and bioactive metabolic derivatives, and/or complexes can be included in membranes encapsulating an edible or potable substance and/or within the edible or potable substance. BCAA can be obtained in commercially manufactured compositions, usually in a ratio of 2 parts L-leucine:1 part L-isoleucine:1 part L-valine. Such compositions commonly are in powdered form. Optionally, the BCAA composition comprises glutamine, and is often commercially identified and/or labeled as BCAA plus glutamine. Daily dosage of BCAA is dependent on the consumer needs. For example, high muscle mass individuals and/or those seeking to build muscle mass may desire higher daily dosage than an “average” consumer. Other consumers such as hospital patients (post-operative patients, trauma patients, burn victims, malignant disease sufferers, etc.) may also desire increased dosages to gain the full health benefits of the BCAA/glutamine composition. If desired, the functional food compositions can provide up to about 500 milligrams (mgs), up to about 1000 mgs, up to about 2000 mgs, up to about 3000 mgs, up to about 4000 mgs, up to about 5000 mgs, up to about 6000 mgs, up to about 7000 mgs, up to about 8000 mgs, up to about 9000 mgs, up to about 10,000 mgs, up to about 12,500 mgs, up to about 15,000 mgs, up to about 17,500 mgs, up to about 20,000 mgs or more of L-leucine in a single serving size. If desired, the functional food compositions can provide up to about 500 milligrams (mgs), up to about 1000 mgs, up to about 2000 mgs, up to about 3000 mgs, up to about 4000 mgs, up to about 5000 mgs, up to about 6000 mgs, up to about 7000 mgs, up to about 8000 mgs, up to about 9000 mgs, up to about 10,000 mgs, up to about 12,500 mgs, up to about 15,000 mgs, up to about 17,500 mgs, up to about 20,000 mgs or more of L-isoleucine in a single serving size. If desired, the functional food compositions can provide up to about 500 milligrams (mgs), up to about 1000 mgs, up to about 2000 mgs, up to about 3000 mgs, up to about 4000 mgs, up to about 5000 mgs, up to about 6000 mgs, up to about 7000 mgs, up to about 8000 mgs, up to about 9000 mgs, up to about 10,000 mgs, up to about 12,500 mgs, up to about 15,000 mgs, up to about 17,500 mgs, up to about 20,000 mgs or more of L-valine in a single serving size. If desired, the functional food compositions can provide up to about 500 milligrams (mgs), up to about 1000 mgs, up to about 2000 mgs, up to about 3000 mgs, up to about 4000 mgs, up to about 5000 mgs, up to about 6000 mgs, up to about 7000 mgs, up to about 8000 mgs, up to about 9000 mgs, up to about 10,000 mgs, up to about 12,500 mgs, up to about 15,000 mgs, up to about 17,500 mgs, up to about 20,000 mgs or more of L-glutamine in a single serving size.

Generally, multivitamins and bioavailable and bioactive metabolic derivatives and/or complexes can be included in membranes encapsulating an edible or potable substance and/or within the edible or potable substance. Concentrated, isolated and combinations of compounds useful as multivitamins are commercially available. Generally, vitamins, minerals and other supplements that can be used in the multivitamin functional food can be, without limitation, ascorbic acid (vitamin C), vitamin B₁₂, biotin, fat soluble vitamins, folic acid, HCA (hydroxycitric acid), inositol, pyruvate, mineral ascorbates, mixed tocopherols, niacin (vitamin B₃), orotic acid, PABA (para-aminobenzoic acid), pantothenates, pantothenic acid (vitamin B₅), pyridoxine hydrochloride (vitamin B₆), riboflavin (Vitamin B₂), synthetic vitamins, thiamine (vitamin B₁, tocotrienols, vitamin A, vitamin D₃, vitamin E, vitamin F (linoleic acid (LA) and alpha-linoleic acid (LNA)), vitamin K₁, vitamin K₂, vitamin oils, vitamin premixes, vitamin-mineral premixes, water soluble vitamins, arsenic, boron, calcium, chloride, chromium, cobalt, copper, fluorine, iodine, iron, magnesium, manganese, molybdenum, nickel, phosphorous, potassium, selenium, silicon, sodium, strontium, sulfur, vanadium, zinc, combinations thereof, etc. If desired, the amount of vitamins, minerals and/or other supplements provided in a multivitamin functional food composition sufficient for an effective daily intake can be, for example, up to 10% of recommended daily allowance (RDA), up to 20% RDA up to 30% RDA, up to 40% RDA, up to 50% RDA, up to 60% RDA, up to 70% RDA, up to 80% RDA, up to 90% RDA, up to 100% RDA, up to 500% RDA, up to 1000% RDA, up to 2000% RDA, up to 3000% RDA, up to 4000% RDA, up to 5000% RDA.

If desired, enzymes and amino acids can be included in the functional food as described herein, and include, but are not limited to, alpha galactosidase, amylase, bromelain, cellulase, papain, peptidase, protease, proteolytic enzymes, superoxide dismutase, trypsin, betaine, casein, glutamic Acid, L-alanine, L-arginine, L-cysteine, L-glutamine, L-glycine, L-histidine, L-lysine, L-methionine, L-ornithine, L-phenylalanine, L-proline, L-taurine, L-threonine, L-tryptophan, L-tyrosine, N-acetly-L-cysteine, protein soluble soy, soy protein isolates, whey protein isolates, combinations thereof, etc.

Uses of BCAA Functional Food Compositions

Functional food compositions described herein are contemplated to provide a functional benefit to a consumer (“functional food”). Functional foods are formulated to provide an important nutritive food source that contributes to the prevention, management and/or treatment of chronic diseases and/or maintenance of a healthy state, as well as provide a source of glutamine and BCAAs for the promotion and maintenance of general health, recovery from surgical procedures, trauma recovery and treatment, wasting associated with critical/malignant disease states, severe burns, and mitigation of disease and disorder treatment. These benefits can be provided to a consumer when new ingredients or more of an existing ingredient are added to a food so that an added health benefit from the food product is gained.

The important role that BCAA and glutamine supplements plays in muscle maintenance is recognized in the sports and athletic industry where high levels of catabolic states are reached during strenuous activity, and so is included as a supplement used for muscle growth and maintenance for weightlifting, bodybuilding, endurance sports and other sports activity.

The functional food compositions as described herein increase the effective delivered concentration of such useful BCAAs and glutamine, and can be formulated for daily dietary supplements, preventative treatment and mitigation of muscle mass loss related thereto.

X. Overview of the Antioxidant Functional Food Compositions

The disclosure relates to functional food compositions that contain compounds when, upon ingestion, can increase the general health and well-being of the consumer. The functional food composition described herein contains an edible or potable substance that is encapsulated in an edible membrane, and further contains an edible antioxidant compound characterized by an oxygen radical absorbance capacity value of at least 1000 per 100 grams, supplementing the antioxidant concentration of a normal diet. The functional food compositions described herein provide a convenient and improved way for individuals to consume antioxidants in combinations and/or sufficient quantities needed to supplement normal diet to treat unhealthy bodily conditions and disease, and increase general health and well-being related thereto.

Generally, functional food compositions are intended to provide a healthy benefit to a consumer. The benefit can be qualitative, quantitative or subjective and include, for example, maintenance and promotion of good health, disease prevention, etc. Some benefits arise from providing an important nutritive food source, vitamins and supplements and/or compounds such as antioxidants that generally or specifically contribute to increased health and wellness. A higher degree of health assists in the prevention, management and treatment of acute or chronic diseases or disorders. This health and wellness improvement or benefit can be provided to a consumer when additional ingredients or more of an existing ingredient are added to a food so that the benefit from the food composition is gained. However, delivery systems for some bioavailable compounds, including those important for supplementing a diet, can reduce the viability of the compounds, resulting in suboptimal quantities of these compounds being made available to the consumer for a particular food composition. Often, the delivery system for a nutritional supplement is in a form insufficient to promote compliance in a particular consumer group (e.g., elderly, infirm, children and young adults), because the delivery system is in a large pill form, powder form with little gustatory appeal, or poor tasting liquids.

The functional food compositions described herein provide for a significant improvement in the delivery, viability and concentration of antioxidant containing compounds useful to a consumer. Within these compositions, the antioxidant containing compounds can be compartmentalized within the edible or potable substance, the encapsulating membrane, or both. Upon mastication and ingestion, the antioxidants are released from the functional food composition to provide an immediately available nutrition and edible antioxidant source. The functional food compositions can thus be designed to have one or more antioxidant containing compounds in an effective combination and concentration desirable for the specific needs of the consumer or patient, for example, the elderly, adult and children consumers, and medical patients.

The functional food compositions described herein are designed to provide antioxidants in biologically effective quantities for promoting general health and well-being in concentrations superior to commonly used products. The compositions are particularly well-suited for providing a larger active ingredient load as well as being amenable for controlling the actual portion size for an intended health benefit. Functional food compositions as described herein can provide up to and above several times the suggested minimum dosages in a sample serving, or provide desired combinations, of antioxidant containing compounds, nutraceuticals and supplements up to and above several times the suggested minimum dosages in a single serving that is both convenient and appetizing. A functional food delivering a combination of antioxidant containing compounds in a single serving composition can result in the cessation or mitigation of a multitude of conditions and diseases, including skin related conditions (e.g., wrinkles, dry skin, brittle nails, hair loss, skin spots, etc.), cardiovascular conditions, urinary tract conditions, etc. The functional food described herein can also supplement dietary imbalance and promote user compliance in the daily consumption of these and other health promoting compounds. In addition, a general maintenance state of health and wellness can result from a healthy diet supplemented with these and other functional foods as described herein.

Population segments that may find useful an antioxidant functional food composition as described herein include the elderly, adults, hospital patients, those with a variety of conditions related to integument, cardiovascular health, urinary tract health, healthy brain function and mental acuity, blood sugar management, visual health, etc.

However, these consumers can become noncompliant for daily intake of health compounds depending on the delivery form of the composition. For example, some health compounds are in pill form with a flavor, texture and overall size (e.g., omega-3 and omega-6 fatty acids, multivitamin supplements, etc.) that often result in reluctance to consume on a scheduled or daily basis. Therefore, a functional food as described herein that delivers a daily concentration of antioxidant containing compounds can overcome non-compliance.

The functional food compositions described herein also enhance the stability, gustatory experience, and delivery of functional biologically active compounds. The functional food compositions contain and protect ingestible/edible substances, such as ice cream, yogurts, etc., in addition to the antioxidant containing compounds, by encapsulating the substance within edible or biodegradable membranes, for which the membranes provide barriers to oxygen and oxygen degradation, diffusion, etc. The edible membranes of these compositions can be formed from various natural polymeric substances that allow the compositions to be easily transported and consumed by a consumer.

Generally, edible compounds that naturally contain high ORAC values include a variety of fruits and vegetables. ORAC values are expressed as the sum of the lipid-soluble (e.g. carotenoid) and water soluble (e.g. phenolic) antioxidant fractions (i.e., “total ORAC”) of the food (fruits, vegetables, spices, etc.) and are reported in micromoles trolox equivalents (TE) per 100 gram sample compared to assessments of total polyphenol content in the fruit and vegetable samples. If desired, combinations of fruits and vegetables can be used together such that the combined ORAC value of the combination is over about 1000 per 100 grams. If desired, the fruits and vegetables, combinations thereof, or individual fractions can be as an extract and/or powdered form.

The benefits of high antioxidant containing foods are exemplified by the so called Mediterranean Diet, which is rich in plant derived bioactive phytochemicals and has been correlated to lower rates of cardiovascular disease. For example, lycopene is a natural carotene found in tomato, an essential component of the Mediterranean diet, and functions at least as an antioxidant scavenger.

Aging, metabolic and hormonal changes related to aging, exposure to environmental conditions (sun exposure, arid environments), etc., can all contribute to changes in general health and wellbeing, and may contribute to systemic disorders and diseases health. Environmental exposure such as ultraviolet (UV) radiation damages skin cells and hair. Over the longer term, UV radiation induces degenerative changes in cells of the skin, fibrous tissue and blood vessels leading to premature skin aging and wrinkles, lentigo, etc. UV exposure directly affects the bilayer making up a cell membrane because it is made up of significant amount of unsaturated lipids that are more prone to attack by free radicals. Damage by free radicals is assumed to be one of the causative factors of aging, inflammation and hyper pigmentation. In fact, the stratum corneum has been found to produce significant concentrations of free radicals under UV exposure, damaging DNA, lipids, proteins and carbohydrates. The oxidization of low density lipoprotein (“bad cholesterol”) is thought to be the primary step leading to its interaction with macrophages inside the arterial wall and the formation of atherosclerotic plaque. Therefore higher levels of blood antioxidants are thought to contribute to mitigating the degree of low density lipoprotein oxidation and plaque formation. Nitric oxide and reactive oxygen species (ROS) including superoxide anion and hydrogen peroxide are highly reactive oxidant molecules produced by the human body through normal metabolism and diet, and also may play a role in certain conditions including the loss of elasticity in skin, development of cancers, etc., in addition to cardiovascular disease. Antioxidants are protective compounds that inactivate ROS and delay or prevent oxidative damage. Medical investigations suggest that regular consumption of antioxidant-containing foods can reduce the risk of cardiovascular diseases and various skin conditions and diseases, providing direct evidence for the potential systemic benefits of a diet rich in antioxidants.

While antioxidants are ubiquitous in plants, cocoa (Theobroma cacao) and tea (Camellia sinensis) have been found to be particularly rich in a class of antioxidants called flavanols. A number of human dietary intervention trials with flavanol-containing cocoa and tea products have demonstrated improvements in systemic function as well as, for example, regulation in blood pressure and conditions or disorders related to oxidative damage. Flavanols are also found in fruits and vegetables and are linked to certain health benefits linked to, for example, coronary heart disease and stroke. As with antioxidants including lycopene and other carotenes, flavanols are thought to take part in mechanisms such as nitric oxide and antioxidant, anti-inflammatory, and antiplatelet effects to improve endothelial function, lipid levels, blood pressure and insulin resistance, particularly with respect to preventing the oxidization of low density lipoprotein, and can contribute to mitigating the effect of free radical damage on the integument, youthful skin appearance, beauty, and maintenance of integument health and well-being.

Various antioxidant containing compounds, supplements, and nutraceuticals can be included in the functional food compositions described herein in quantities sufficient to correlate with recommended daily allowances, for the maintenance of general health, treatment and prevention of systemic disorders, diseases and related conditions, and are beneficial supplementations to normal dietary intake. Other antioxidant compounds include bioflavonoids such as esveratrol, quercetin, rutin, catechin, epichatechins, proanthocyanidins, and combinations thereof. These bioflavonoids can be derived from various plants including, for example, Camellia sinensis, Theobroma cacao (cocoa flavanols) and species, varieties, cultivars, thereof. Bioflavonoids can also be derived from or found in various plant extracts including, but not limited to, acai berry extract, raspberry extract, cranberry extract, pomegranate extract, plum extract, cherry extract, rosemary extract, etc. Isoflavones are used as membrane particles, including, but not limited to, genistein, daidzein, biochanin A, and formononetin. Carotenes contemplated for use as antioxidant compounds include α-carotene, β-carotene, γ-carotene, δ-carotene, ε-carotene, lycopene, cryptoxanthin, lutein, zeaxathin, apocarotenal astaxanthin, canthaxanthin, lutein/lutein esters, etc. Lycopene is one of the most potent antioxidants, with activity twice as high as that of β-carotene and tenfold higher than α-tocopherol. Fruits and vegetables that are high in lycopene include gac, tomatoes, watermelon, pink grapefruit, pink guava, papaya, seabuckthorn, wolfberry,goji, a berry relative of tomato, and rosehip. The lycopene content of tomatoes depends on species and increases as the fruit ripens. Vitamins are contemplated for use in the functional food described herein and include vitamin C, vitamin E and vitamin A, all having high antioxidant capacity. All antioxidant compounds contemplated for use herein include bioavailable forms of antioxidants and bioactive derivatives thereof, salts thereof, and bioactive metabolic intermediates thereof.

Other compounds can be used exclusively or in combination with the antioxidant containing compounds of the functional food compositions described herein. If desired, dietary fibers can also be incorporated within the functional food composition as described herein. Dietary fiber is the edible parts of plants or analogous carbohydrates that are resistant to digestion and absorption in the human small intestine, with complete or partial fermentation in the large intestine. Dietary fiber includes polysaccharides, oligosaccharides, lignin, and associated plant substances. Dietary fibers promote beneficial physiologic effects including laxation, and/or constipation relief, and/or blood cholesterol attenuation, and/or blood glucose attenuation. Dietary fibers also enhance a satiety effect, wherein food portions can become effectively smaller while maintaining a feeling of fullness after a meal.

Nutraceuticals are generally thought of as food or food product that reportedly provides health and medical benefits, including the prevention and treatment of disease, and can be defined as a product isolated or purified from foods that is generally sold in medicinal forms not usually associated with food. A nutraceutical may have a physiological benefit or provide protection against chronic disease. Such nutraceutical products may range from isolated nutrients, vitamins and minerals, other dietary supplements, and herbal products. With recent developments in cellular-level nutraceutical agents, researchers, and medical practitioners are developing templates for integrating and assessing information from clinical studies on complementary and alternative therapies into responsible medical practice.

Functional Food Compositions with Antioxidants

In general, vegetables, fruits, spices, berries and legumes, with high antioxidant ORAC values for use as an antioxidant containing compound can include, without limitation, broccoli, broccoli sprout extract, tomato, carrots, kale, brussel sprouts, cocoa beans, chokeberry, red beans, blueberries, kidney beans, pinto beans, cranberry, artichoke hearts, blackberry, prune, raspberry, strawberry, red delicious and granny smith apples, black plum, russet potato, black bean, plum, gala apple, pecans, sweet cherry, pomegranate, blackcurrant, figs, guava, oranges, mango, grapes, clove, cinnamon, oregano, turmeric, cumin, parsley, basil, curry powder, mustard seed, ginger, pepper, chili powder, paprika, garlic, coriander, cardamom, sage, thyme, marjoram, tarragon, peppermint, oregano, savory, basil, dill weed, pears, peaches, raisins, cabbage, artichokes, broccoli, asparagus, avocado, beetroot spinach, collard greens, etc. If desired, the ORAC value provided the antioxidant containing compound and/or mixtures and combinations of compounds is at least about 1000 per 100 grams, at least about 2000 per 100 grams, at least about 3000 per 100 grams, at least about 4000 per 100 grams, at least about 5000 per 100 grams, at least about 6000 per 100 grams, at least about 7000 per 100 grams, at least about 8000 per 100 grams, at least about 9000 per 100 grams, at least about 10,000 per 100 grams, at least about 15,000 per 100 grams, at least about 20,000 per 100 grams, at least about 25,000 per 100 grams, at least about 30,000 per 100 grams, at least about 35,000 per 100 grams, at least about 40,000 per 100 grams, at least about 45,000 per 100 grams, at least about 50,000 per 100 grams, at least about 55,000 per 100 grams, at least about 60,000 per 100 grams, at least about 65,000 per 100 grams, at least about 70,000 per 100 grams, at least about 75,000 per 100 grams, at least about 80,000 per 100 grams.

In general antioxidants contemplated for use for the functional food composition as described herein can include, without limitation carotenes, bioflavonoids (flavonoids, isoflavones, neoflavonals), tocopherol, tocotrienol, lipoic acid, melatonin, superoxide dismutase, coenzyme Q10, alpha lipoic acid, vitamin A, chromium biotin, selenium and ascorbic acid. Carotenes contemplated for use as membrane particles include α-carotene, β-carotene, γ-carotene, δ-carotene, ε-carotene, lycopene, cryptoxanthin, lutein, zeaxathin, apocarotenal astaxanthin, canthaxanthin, lutein/lutein esters, etc. If desired, the amount of carotene in the functional food composition serving as described herein can be up to about 1 milligram (mg), up to about 5 mg, up to about 10 mg, up to about 15 mg, up to about 20 mg, up to about 25 mg, up to about 30 mg, up to about 35 mg, up to about 35 mg, up to about 40 mg, up to about 45 mg, up to about 50 mg, up to about 60 mg, up to about 70 mg, up to about 80 mg, up to about 90 mg, up to about 100 mg. Bioflavonoids used for the functional food composition described herein include esveratrol, quercetin, rutin, catechin, epichatechins, proanthocyanidins, and combinations thereof. Bioflavonoids can be derived from various plants including, for example, Camellia sinensis, Theobroma cacao (cocoa flavanols) and species, varieties, cultivars, thereof. Bioflavonoids can also be derived from or found in various plant extracts including, but not limited to, acai berry extract, raspberry extract, cranberry extract, pomegranate extract, plum extract, cherry extract, rosemary extract, etc. Isoflavones are used as membrane particles, including, but not limited to, genistein, daidzein, biochanin A, and formononetin. If desired, the amount of bioflavonoid in the functional food composition serving as described herein can be up to about up to about 50 mg, up to about 100 mg, up to about 150 mg, up to about 200 mg, up to about 250 mg, up to about 300 mg, up to about 350 mg, up to about 400 mg, up to about 450 mg, up to about 500 mg, up to about 600 mg, up to about 700 mg, up to about 800 mg, up to about 900 mg, up to about 1000 mg.

Total ORAC provided by the antioxidant containing compound per serving of the functional food described herein can be at least about 2000, at least about 3000, at least about 4000, at least about 5000, at least about 6000, at least about 7000, at least about 8000, at least about 9000, at least about 10,000, at least about 15,000, at least about 20,000, and higher.

Dietary fibers for the functional food composition as described herein can include, both soluble and insoluble dietary fibers, including, but not limited to, galacto-oligosaccharides, inulin and oligofructose (fructo-oligosaccharide), isomalto-oligosaccharides, lactulose, lactosucrose, transgalacto-oligosaccharides, soybean oligosaccharides, tagatose, xylo-oligosaccharides, and combinations thereof. The dietary fiber can be an oligosaccharide fructan including, but not limited to, fructo-oligosaccharide and derivatives thereof of inulin (poly-D-fructose, itself derived from various plant products including bananas, onions, chicory root, garlic, asparagus, barley, wheat, jicama, leeks, etc.) or from transfructosylation action of a β-fructosidase as used, for example, by Aspergillus sp. metabolically active on sucrose. Dietary fibers can be derived from plants and plant products including, but not limited to, artichoke, dandelion greens, bran, flour, legumes, oats, citrus fruits, apples, and root vegetables, cane sugar, etc. If desired, the amount of total dietary fiber per function food serving can be less than about 1 gram, less than about 2.5 grams, less than about 5 grams, less than about 7.5 grams, less than about 10 grams, less than about 12.5 grams, less than about 15 grams, less than about 17.5 grams, less than about 20 grams.

Uses of Antioxidant Functional Food Compositions

Functional food compositions described herein are contemplated to provide a functional benefit to a consumer (“functional food”). Functional foods are formulated to provide an important nutritive food source that contributes to the prevention, management and/or treatment of chronic diseases and/or maintenance of a healthy state, as well as provide a source of antioxidant for the promotion and maintenance of general health, and mitigation of disease and disorder treatment. These benefits can be provided to a consumer when new ingredients or more of an existing ingredient are added to a food so that an added health benefit from the food product is gained.

Supplementing a diet with antioxidant containing compounds deliverable in the functional foods as described herein is useful for prevention and treatment of a variety of conditions and disorders, particularly those related to free-radical damage, as well as for general health and wellness maintenance.

The functional food compositions as described herein increase the effective delivered concentration of such useful antioxidants, and can be formulated for daily dietary supplements, preventative treatment and mitigation of other disorders related to environmental damage and aging.

XI. Overview of the β-Glucan/Probiotic Functional Food Composition

The disclosure relates to functional food compositions that contain a β-glucan and a probiotic when, upon ingestion, promote immune responses and immunity related health and well-being. The functional food composition contains an edible or potable substance that is encapsulated in an edible membrane, and further contains a probiotic and a β-glucan. The functional food compositions described herein provides a convenient and improved way for individuals to consume probiotics and β-glucan in quantities needed to promote immunological health and well-being.

Generally, functional food compositions are intended to provide a healthy benefit to a consumer. The benefit can be qualitative, quantitative or subjective and include, for example, health promotion, disease prevention, etc. Some benefits include providing an important nutritive food source, vitamins and supplements, β-glucan or probiotics, which contribute to the prevention, management and treatment of chronic diseases or disorders. This health improvement benefit can also be provided to a consumer when additional ingredients or more of an existing ingredient are added to a food so that the benefit from the food product is gained. However, delivery systems for some beneficial health compounds including β-glucan and probiotics often have reduced bioavavailability, resulting in suboptimal quantities of these compounds being made available to the consumer.

The functional food compositions described herein provide for a significant improvement in the delivery, viability and concentration of β-glucan and probiotic compounds useful to a consumer. Within these compositions, the β-glucan and probiotic compounds can be compartmentalized within the edible or potable substance, the encapsulating membrane, or both. Upon mastication and ingestion, the β-glucan and probiotic are released from the functional food composition to provide an immediately available nutrition and edible source. The functional food compositions can thus be designed to have one or more β-glucan and probiotic compounds in an effective combination and concentration desirable for the specific needs of the consumer or patient, for example, the elderly, adult and children consumers, and medical patients.

The functional food compositions described herein are designed to provide β-glucan and probiotic compounds in biologically effective quantities superior to commonly used products. The compositions are particularly well-suited for providing a larger active ingredient load as well as being amenable for controlling the actual portion size for an intended health benefit. Functional food compositions as described herein can provide up to and above several times the suggested minimum dosages in a sample serving, or provide combinations of β-glucan and probiotic compounds, nutraceuticals and supplements up to and above several times the suggested minimum dosages in a single serving that is both convenient and appetizing. For example, an eight ounce serving of commercial yogurt in plastic containers commonly provides a microbial load of less than 1 billion colony forming units (BCFU), far below a bacterial load level sufficient to provide health benefits. The compositions described herein can be designed to provide up to 100 BCFU's. A functional food delivering a combination of β-glucan and probiotics in a single serving composition can contribute to the prevention, cessation or mitigation of a multitude of health related conditions and diseases. In addition, a general state of health and wellness can result from a healthy diet supplemented with β-glucan and live and/or heat killed probiotic compounds.

These functional food compositions also enhance the stability, gustatory experience, and delivery of functional biologically active compounds. Functional food compositions contain and protect ingestible/edible substances, such as ice cream, yogurts, etc., and probiotics or β-glucan by encapsulating the substance within edible or biodegradable membranes. The edible membranes of these compositions can be formed from various natural polymeric substances allowing different compositions to be easily transported and consumed.

Considerations for the choice of probiotic or probiotic combinations for use in the function food compositions are that, if desired, the probiotic remain viable until ingestion in a biologically effective concentration, and the probiotic be able to confer a health benefit to and/or use for treatment of a gastro-intestinal, immune or other systemic disorder in the host organism.

In some instances, heat-treated probiotics or probiotics that are otherwise rendered non-viable, and/or immune enhancing constituents derived from heat killed or non-viable probiotics, are preferred and are used in the compositions described herein. For example, heat killed Lactobacillis species have been shown to augment innate and acquired immunity in mammals.

In all instances, it is desirable that these food compositions (product) include effectual probiotics to enhance the immune response otherwise confer a health benefit to a host organism ingesting the probiotic.

β-glucans are major biological structural components of yeasts, fungi and certain bacteria. β-glucans are polysaccharides that can be obtained from natural sources, and consist of a backbone of (1,3)-β-glycosidic linked D-glucose subunits, and having regularly or irregularly placed β-(1,6)-linked (1,3)-β glycosidic linked D-glucose side chains of various lengths. The various side chain combinations can affect immunological effects due to resulting changes in the β-glucan solubility, mass, tertiary structure, branching patterns and electrostatic charge. Innate immune responses to invading pathogens depend primarily on the recognition of pathogen-associated molecular patterns (PAMPs) by pattern recognition receptors (PRR). β-glucans are known as a major PAMP for the PPR-mediated sensing of fungal infection. β-glucans polymers are able to bind to the dectin receptor (an important PRR for beta-glucans), to activate the dectin-1 signaling to enhance phagocytosis, presentation of macrophages and dendritic cells, and induces the adaptive immune response.

Generally, the probiotics and β-glucans as used herein are taken 1) to enhance the physical well-being or state of health of the end user, 2) as a health related supplement, 3) as supplements for enhancing immune response or mitigation of autoimmune responses, and/or 4) as supplements to maintain a healthy immune system. Diets supplemented with probiotics and β-glucans can also add to a higher quality or perceived quality of the health state of the end user.

Health benefit aspects contemplated for use of the β-glucans and probiotic functional food compositions described herein include relief from intestinal inflammation, beneficial pathogen and antigen response, decreased time of infection, decreased severity of infection, prevention of infection, etc. Health and wellness preventative treatment with probiotics includes decreased immunological response time, enhanced humoral immune response, stimulation of non-specific host resistance to microbial pathogens, and modulation of the host's immune responses to potentially harmful antigens, and regulation of hypersensitivity reactions, including statistically significant decreases in the onset of upper respiratory tract infections, demonstrated augmentation of acquired immunity, reduction in the severity of cold and flu symptoms, and reduction in nasal and ocular symptoms due to ragwood allergy. Additionally, β-glucans and probiotics may beneficially affect the host by augmenting the host intestinal microbial population beyond the amount already existing, thus possibly inhibiting unhealthy or harmful pathogens.

As described in more detail herein, the common probiotics include lactic acid bacteria, bifidobacteria, yeasts and bacilli. Often these probiotics are consumed as part of a fermented food source, (e.g., yogurt, soy yogurt, etc.) with added live microbiological cultures.

Measurable outcomes for general health maintenance can be determined quantitatively and/or qualitatively by monitoring, for example, a patient's or consumer's change in composition of intestinal microflora, decreased incidence of pathological infection, decreased intensity of pathological infection, decreased recovery time from pathological infection, and increased possibly feelings of well-being. For pretreatment/prevention of diseases, conditions and/or syndromes, the consumer or patient may show milder symptoms or no symptoms. In acute conditions for which the consumer or patient is using the functional food composition, for example diarrheal conditions (i.e. traveler's, antibiotic or viral) or infection from viruses related to the common cold, the outcome would be faster resolution and reduction in severity for treatment. In general, a reduction in discomfort related to infection or an immune response, and improvement in quality of life and ability to perform activities of daily living are all indicators of the health benefits when consuming the functional food compositions described herein.

β-Glucan and Probiotic Compositions

Common probiotics include lactic acid bacteria, bifidobacteria, yeasts and bacilli, and are often consumed as part of a fermented food source, (e.g., yogurt, soy yogurt, etc.) with added live microbiological cultures. Probiotics in concentration and/or type contained within the functional food compositions included herein may be used in addition to natural levels of probiotics in the food source. Alternatively, probiotics as described herein may be used in food sources with no known natural probiotic levels. Inactivated probiotics, for example heat or chemically inactivated probiotics, having an antigenic effect may be used in the compositions described herein.

Probiotics for use in the foods described herein include, but are not limited to, Aspergillus niger, Aspergillus oryzae, Bacillus coagulans, Bacillus lentus, Bacillus licheniformis, Bacillus pumilus, Bacillus subtilis, Bacteroides amylophilus, Bacteroides capillosus, Bacteroides ruminocola, Bacteroides suis, Bifidobacterium animalis, Bifidobacterium bifidum, Bifidobacterium breve, Bifidobacterium infantis, Bifidobacterium licheniformis, Bifidobacterium longum, Bifidobacterium pseudo longum, Bifidobacterium subtilus, Bifidobacterium thermophilum, Enterococcus cremoris, Enterococcus diacetylactis, Enterococcus faecium, Enterococcus thermophilus, Lactobacillus acidophilus, Lactobacillus amylovorus, Lactobacillus ansporogenes, Lactobacillus brevis, Lactobacillus buchneri, Lactobacillus bulgaris, Lactobacillus casei, Lactobacillus caucasicus, Lactobacillus cellobiosus, Lactobacillus crispatus, Lactobacillus curvatus, Lactobacillus delbrueckii, Lactobacillus farciminis, Lactobacillus fermentum, Lactobacillus gallinarum, Lactobacillus gasseri, Lactobacillus GG, Lactobacillus helveticus, Lactobacillus johnsonii, Lactobacillus kefir, Lactobacillus lactis, Lactobacillus plantarum, Lactobacillus reuteri, Lactobacillus rhamnosus (casei), Lactobacillus salivarius, Lactococcus lactis, Leuconostoc mesenteroides, Pediococcus acidilactici, Pediococcus acidilacticii, Pediococcus cerevisiae, Pediococcus pentosaceus, Propionbacterium freudenreichii, Propionibacterium shermanii, Saccharomyces boulardii, Saccharomyces cerevisiae, Shewanella colwelliana, Shewanella olleyana, Shewanella putrefaciens, Streptococcus cremoris, Streptococcus faecium, Streptococcus infantis, Streptococcus thermophilus, Vagococcus fluvialis, Bacillus coagulans GBI-30, 6086, Bifidobacterium animalis DN 173 010, Bifidobacterium animalis subsp. lactis BB-12, Bifidobacterium breve Yakult, Bifidobacterium infantis 35624, Bifidobacterium lactis HN019 (DR10), Bifidobacterium longum BB536, Bifidobacterium longum subsp. infantis 35624, Enterococcus LAB SF 68, Escerichia coli Nissle 1917, Lactobacillus acidophilus NCFM, Lactobacillus acidophilus LA-5, Lactobacillus acidophilus NCFM, Lactobacillus casei subspecies casei, Lactobacillus casei DN-114 001, Lactobacillus casei CRL431, Lactobacillus casei F19, Lactobacillus casei Shirota, Lactobacillus delbrueckii subspecies bulgaricus, Lactobacillus delbrueckii subspecies lactis, Lactobacillus paracasei St11 (or NCC2461), Lactobacillus johnsonii La1 (Lj1), Lactobacillus johnsonii NCC533, Lactobacillus plantarum 299v, Lactobacillus rhamnosus ATCC 53013 (LG), Lactobacillus rhamnosus LB21, Lactobacillus reuteri ATCC 55730, Lactobacillus salivarius (UCC118), Lactobacillus reuteri SD2112, Lactococcus lactis L1A, Lactococcus lactis subspecies lactis, Lactococcus lactis subspecies lactis biovariant diacetylactis, Lactococcus lactis subspecies cremoris, Leuconostoc mesenteroides subspecies cremoris, Leuconostoc mesenteroides subspecies dextranicum, Lactobacillus reuteri Protectis (DSM 17938, daughter strain of ATCC 55730), Saccharomyces cerevisiae boulardii, Saccharomyces cerevisiae boulardii (lyo), Lactobacillus bulgaricus, Streptococcus thermophiles, Bifidobacterium spp, and any other strains, derivatives, and combinations thereof, including, but not limited to, Lactobacillus rhamnosus GR-1 & Lactobacillus reuteri RC-14, Lactobacillus acidophilus CL 1285 & Lactobacillus casei Lbc80r, Lactobacillus rhamnosus (ATT SD5675) & Lactobacillus acidophilus (STCC SD5221), VSL #3 (a combination of 1 strain of Streptococcus thermophiles, four strains of Lactobacillus species, & three Bifidobacterium species strains from Sigma-Tau Pharmaceuticals), Lactobacillus acidophilus NCFM & Bifidobacterium bifidum BB-12, Lactobacillus acidophilus CUL60 & Bifidobacterium bifidum CUL20, Lactobacillus acidophilus CL1285 & Lactobacillus casei LBC80R, Lactobacillus helveticus R0052 & Lactobacillus rhamnosus R0011, Bacillus clausii (strains O/C, NR, SIN and T), Lactobacillus plantarum HEAL 9 & Lactobacillus paracasei 8700:2, other genetic variants thereof, and combinations and other combinations thereof. In certain instances the probiotic may be heat killed or otherwise rendered non-viable and included in the functional food product described herein. In still other instances, constituents of the heat killed or otherwise rendered non-viable probiotic may be used in the functional foods described herein.

When the dosage or intake of the effective dose of the active ingredients of the functional food composition are indicated by the number (concentration) of the probiotic, it is preferable that the intake is about 0.5-1.0 (BCFU) or more per day, more preferably about 10 BCFU's or more per day, more preferably about 50 BCFU's or more per day, more preferably about 100 BCFU's or more per day. The concentration of probiotic to be contained in an individual product serving is determined according to the amount of servings ingested per day. The concentration of probiotic present in the functional food composition can be less than about 1 BCFU's, about 1 to about 2 BCFU's, about 3 to about 5 BCFU's, about 5 to about 10 BCFU's, about 10 to about 20 BCFU's, about 20 to about 30 BCFU's, about 30 to about 40 BCFU's, about 40 to about 50 BCFU's, about 50 to about 60 BCFU's, about 60 to about 70 BCFU's, about 70 to about 80 BCFU's, about 80 to about 90 BCFU's, about 90 to about 100 or more BCFU's.

When the dosage or intake of the effective dose of the active ingredients of the functional food composition are indicated by the mass of the probiotic, it is preferable that the intake is about 1 mg to about 10 mg, about 10 mg to about 20 mg, about 20 mg to about 30 mg, about 30 mg to about 40 mg, about 40 mg to about 50 mg, about 50 mg to about 75 mg, about 75 mg to about 100 mg, about 100 mg to about 150 mg, about 150 mg to about 200 mg, about 200 mg to about 250 mg, about 250 mg to about 300 mg, about 300 mg to about 350 mg, about 350 mg to about 400 mg, about 400 mg to about 450 mg, about 450 mg to about 500 mg, about 500 mg to about 600 mg, about 600 mg to about 700 mg, about 700 mg to about 800 mg, about 800 mg to about 900 mg, about 900 mg to about 1000 mg, or more.

In the functional food compositions described herein, β-glucans to be used in the compositions can be derived or obtained from yeasts, fungi, certain bacterial species, plants such as seaweed, or combinations thereof. For example, yeasts such as baker's yeast (Saccharomyces cerevisiae), cereal grains including barley, rye, wheat, oats, and mushrooms (for example shiitake, maitake, reishi, shimeji and oyster varieties) are all common sources of β-glucan. Specifically, (1,3)-(1,6)-β-glucan has been shown to have clinical effect in boosting immune responses; however compositions with (1,3) β-glucan have also been shown to enhance immune responses. In certain embodiments, purified forms of (1,3)-(1,6)-β-glucan, for example from Saccharomyces cerevisiae, are used in the functional food composition.

Concentrations of the β-glucan used for an effective dose depend on the purity of the β-glucan, but can be daily doses of up to about 1 milligram (mg), 2 mgs, 5 mgs, 10 mgs, 20 mgs, 30 mgs, 40 mgs, 50 mgs, 75 mgs, 100 mgs, 200 mgs, 300 mgs, 400 mgs, 500 mgs, 600 mgs, 700 mgs, 800 mgs, 900 mgs, 1000 mgs, 1500 mgs, 2000 mgs, 2500 mgs, 3000 mgs, 3500 mgs, 4000 mgs, 4500 mgs, 5000 mgs. In certain embodiments, concentrations are desired to be about 200 mg to about 300 mg.

If desired, additional compounds are included in the functional food composition to improve or maintain healthy conditions in the digestive tract and/or enhance or modify the microflora and ecological biochemistry to optimize the effects of the probiotic and β-glucan composition. Additional compounds can be insoluble fibers and/or soluble fibers generally not specifically utilized by the probiotic; probiotic extracts (e.g., probiotic exudates and/or non-viable micro-organisms); digestive enzymes including, but not limited to, pancreatin, pancrelipase, papain, pepsin, diatase, ox bile, lactase, protease, amylase, lipase, bromelain, cellulose, malt diastase, glucoamylase, hemicellulose, beta-gluconase, phytase, trypsin, aminopeptidase, chymotrypsin, carboxypeptidase, elastase, maltase, sucrose, etc.; supplements including, but not limited to, glutamine, peppermint, artichoke, ginger, fennel, licorice root, anise seed; and compounds useful for immune health including, but not limited to, vitamins (A, E, C, D); minerals (zinc, selenium); plant derived materials such as Echinacea, ashwaganda, elderberry, etc.; and yeast extracts.

Uses of β-Glucan/Probiotic Functional Foods

Edible transport compositions described herein are contemplated to provide a functional benefit to a consumer (“functional food”). Functional foods are formulated to provide an important nutritive food source that contributes to the prevention, management and/or treatment of chronic diseases, pathogenic infections and infectious conditions, and autoimmune regulation. This benefit can be provided to a consumer when new ingredients or more of an existing ingredient are added to a food so that an added health benefit from the food product is gained. Probiotics and β-glucans are useful for a variety of health related conditions as well as general health maintenance. Immune system effectiveness is enhanced with the combination of probiotic/β-glucan intake. The edible transport compositions as described herein increase the effective delivered concentration of such useful probiotics/β-glucan, and can be formulated for daily dietary supplements, preventative treatment of pathogenic conditions, treatment of nosocomial infections, for clinical applications treating systemic disease states, etc.

Probiotics and β-glucans are have been used as separate indications by physicians and nutritionists to treat, for example, candidiasis, infections by opportunistic bacteria and viruses establishing after trauma, stress, as an antioxidant, or the establishment of normal flora after treatment with antibiotics. In the functional foods described herein, it is preferred that combinations of live and/or non-viable probiotic are used in combination with a β-glucan in the final nutritional functional food product.

In some embodiments, the functional food compositions are used to assist in preventing or mitigating radiation burns. Often the probiotics are recommended by physicians and nutritionists to treat, for example, candidiasis, infections by opportunistic bacteria establishing after trauma, or the establishment of normal flora after treatment with antibiotics. In certain embodiments the functional food described herein can be used to support a cancer treatment regimen (being effective in promoting macrophage generation targeting cancer cells) or for the promotion of the generation of immunocytes in bone marrow.

Probiotics and β-glucans may also play a role in benefitting the immune system by, for example, competing against pathogens though competitive inhibition (e.g. opportunistic bacteria such as C. difficile) via enhancing natural flora, enhancing phagocytosis by white blood cells, and enhancing or increasing the production of certain antibodies and/or T-lymphocytes. Modulation of the immune system by regulating inflammatory and hypersensitivity responses related to cytokine regulation and function is also found to be correlated to intake of probiotics and β-glucans, as is the management of diarrhea and inflammatory bowel disease.

The functional food described herein may play a role in the treatment for systemic disorders such as high cholesterol and high blood pressure. Ingestion of sufficient levels of probiotics is correlated with lowering cholesterol through a mechanism thought to be involved with the breakdown of bile in the intestinal tract to a metabolite that is unable to be reabsorbed. Additionally, reduction in blood pressure has been correlated with intake of fermented dairy products. Thus, it is believed that angiotelisin-converting-enzyme inhibitors or inhibitor like peptides are produced by the probiotic. Maintenance treatment in a normal daily diet (1-10 BCFU's) can be useful for management of systemic disorders such as high cholesterol and/or high blood pressure.

### Membranes Encapsulating Compositions

Embodiments of transport system described herein can have, e.g., varying shell or membrane thickness, one or more of a variety of chemical constituents, varying numbers of membranes, various consumable payloads, various shapes, and are constructed from various shell/membrane properties to provide a variety of flavors and textures and membrane characteristics. See, for example, PCT International Publication No. WO 2011/103594 and PCT International Application No. PCT/US2013/023500, which are incorporated herein by reference in their entirety.

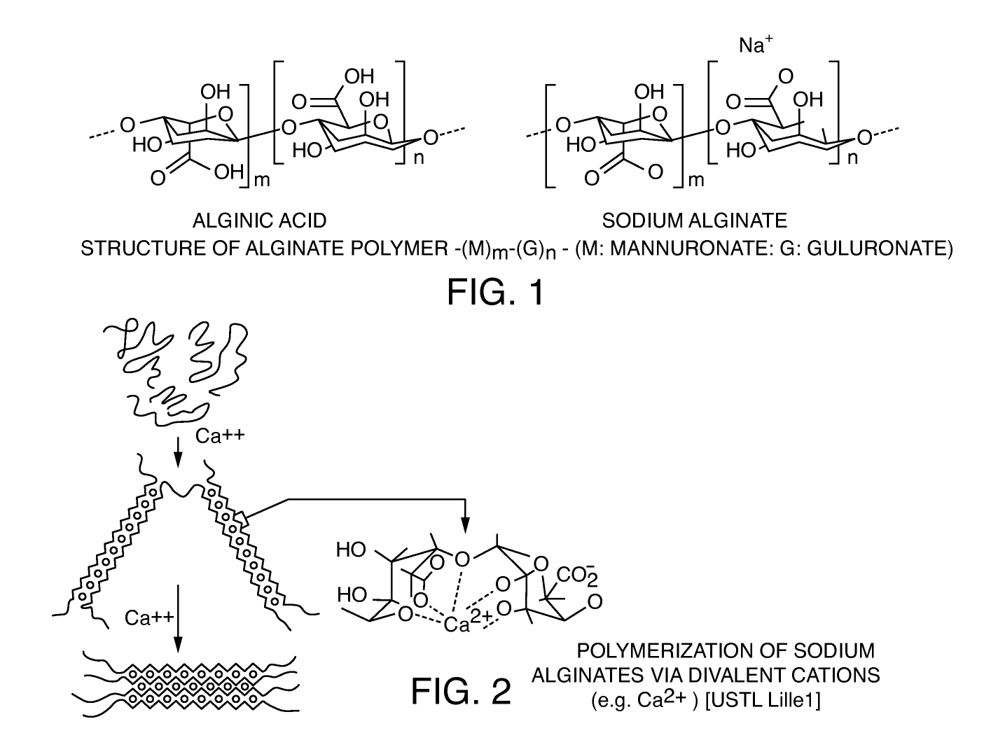

Membranes and shells of the functional food compositions may be made by using any one of many edible and/or biodegradable polymers. FIG. 1 illustrates alginate (alginic acid) as an example of a polymer that can be used in forming a membrane of transport systems. Alginate is an anionic, polymeric polysaccharide, widely present in the cell walls of brown algae. It is a copolymer -(M)ₙ-(G)ₙ- segments composed of mannuronate M (mannuronic acid) and guluronate G (guluronic acid) monomeric subunits. The values of m and n, the ratio m/n, and the space distribution between M and G (i.e. presence of consecutive G-subunits and M-subunits, or randomly organized subunits) all play key roles in the chemical and physical properties of the final membrane. To induce rapid gelation by electrostatic cross-linking, the naturally present Na⁺ ions are removed and replaced by divalent cations (e.g., Ca²⁺ or another multi-valent cation such as Mg²⁺; FIG. 2).

Generally, membrane encapsulated compositions are constructed that use various particles, particulates and polymers, in combination or separately, to create desired properties of strength, stability, permeability, edibility and biodegradability for the transport systems to be easily moved and consumed.

For example, functional food compositions can be encased in a polysaccharide membrane, for example, an alginate membrane. Ingestible particles can be embedded in the membrane to improve the physical, chemical and/or physico-chemical performance characteristics suitable for particular payloads (i.e. edible or potable substances), including, but not limited to, diffusion characteristics, pore size of the membrane, elasticity, etc. When particles are charged and possess the same charge state as other membrane polymers or particulates, membrane component concentrations can vary (for example, decreasing the membrane polymer concentration and increasing the membrane particulate concentration) while maintaining or optimizing membrane performance. When particles carry the opposite charge state as alginate polymers or particulates, the need for a calcium solution or another multivalent ion can be minimized or eliminated by using particles to bind with alginates or another charged polymer. For non-alginate based systems, combinations of homogenous particles can be used to encapsulate the edible material, or can be used in combination with polymers at lower weight %-by-mass than the particles (for example, less than 80%, less than 70%, less than 60%, less than 50%, less than 40%, less than 30%, less than 20%, less than 10% polymer). In some applications, a thinner membrane is desirable to encapsulate a larger quantity of ingestible material, which may have further advantages of taste and texture. Particles contemplated herein include large food particles, for example greater than 1 millimeter (linseeds, sesame seeds, poppy seeds, chia seeds, chopped or pulverized foods including fruits, fruit skins, vegetables, etc.), small grains, and pulverized seeds, nuts, etc. In some applications, compositions use particulates less than about 1 millimeter.

Generally, polysaccharide polymers are used as the membrane polymer. Polysaccharide polymer based membranes are porous, with porosity determined by the chemical content and 2- and 3-dimensional geometry of the polymeric structure of the membrane, for example the structure of the polysaccharide chain. As described herein, various physical and chemical characteristics of the particulates are matched to the membrane structure and chemistry to achieve a desired effect, such as increased impermeability, elasticity, membrane strength-to-weight ratio, color, syneresis, etc. The membrane can have a strength sufficient to support a volume of water, for example 1 cubic centimeter (cc), 5 cc, 10 cc, 20 cc, 30 cc, 40 cc, 50 cc, 100 cc, 200 cc, 300 cc, 400 cc, 500 cc, 600 cc, 700 cc, 800 cc, 900 cc, 1000 cc, 1200 cc, 1400 cc, 1600 cc, 1800 cc, 2000 cc, 3000 cc, 4000 cc, 5000 cc, 6000 cc, 7000 cc, 8000 cc, 9000 cc, 10000 cc, or more, without rupturing, and when handled or transported. The membrane is preferably a continuous membrane, fully encapsulating an edible or potable substance such that the edible or potable substance is fully contained and does not leak out from within encapsulation due to mechanical stress, rupture, shearing, etc. The cross-linked matrix can have a thickness from about 10 microns to about 200 millimeters. The edible or potable substance can be coated and/or embedded in a plurality of membranes.

The particulates used for the membrane can be about 0.01 microns, at about 0.1 microns, at about 0.1 to 1.0 microns, at about 0.1 to 10 microns, at about 0.1 to 100 microns, at about 0.01 to about 1 millimeter or to about 3 millimeters, or at about 0.1 to about 1 millimeter or to about 3 millimeters. The size of the particulates may be important for embedment characteristics into the porous structure of the membrane.

In some applications, it may be desirable that some of the particulates are incorporated into the membrane and a layer of particulates remain unincorporated, forming a layer next to a membrane or between two or more membrane layers. The additional particulate layer can be engineered to alter, for example, permeability, elasticity, strength, durability, syneresis, hygroscopy, hydrophobicity, etc., changes across and within membrane layers. Thus, the chemical nature of the particulates, for example if a hydrophobic particulate is used, can enhance the impedance of liquid diffusion across an inner layer to an outer layer surface boundary. In other aspects, particulates can be layered so that the particulate layer has multiple effects, for example an inner impermeability layer, a middle flavor/texture/payload (e.g. a pharmaceutical or supplement) layer, and an outer strength improving layer.

Particles as described herein can be both in the edible or potable substance and in the membrane. The particles in the membrane and in the edible or potable substance can be the same particles or different particles, can be uniform size or exhibit a range of sizes, and/or exhibit similar or different physical-chemical characteristics.

Various membrane polymers are contemplated for use in the membrane forming layers. Polysaccharide polymers contemplated herein include, but are not limited to, shellac, various fibers and hydrocolloids such as alginate, an agar, a starch, a gelatin, carrageenan, xanthan gum, gellan gum, galactomannan, gum arabic, a pectin, a milk protein, a cellulosic, gum tragacanth and karaya, xyloglucan, curdlan, a cereal β-glucan, soluble soybean polysaccharide, a bacterial cellulose, a microcrystalline cellulose, chitosan, inulin, an emulsifying polymer, konjac mannan/konjac glucomannan, a seed gum, and pullulan. Combinations of these polysaccharides are also contemplated herein.

Other membrane compounds considered for use as structure forming compounds to modify or be used in combination with a polymer-based membrane (for example, a membrane consisting of a polysaccharide) include bagasse, tapioca, chitosan, polylactic acid, processed seaweed, chocolate, starch, gum arabic, cellulose based fibers, natural and synthetic amino acids and polymers thereof, proteins and sugars/sugar derivatives, etc. Combinations of these compounds and compositions are also contemplated herein.

One or more of a variety of ions can be used to polymerize the membrane and for related chemical processes. In, for example, the alginate polysaccharide membrane, ions are used to form cross-linkages between and among individual polymer strands. Various ion/counter ion salt complexes are contemplated for use herein, including, but not limited to, divalent cations such as calcium, potassium, magnesium, manganese, iron, zinc; trivalent cations including, but not limited to, manganese and iron; and salts thereof including, but not limited to, calcium lactate and calcium chloride.

In certain applications micelles can be formed within membranes and between membrane layers and/or between the inner membrane and the edible or potable substance. Micelles can alter the taste experience or mouth feel for the final encased product. Additionally, micelles engineered into the final membrane coated product may contain other ingestibles including sweeteners, flavors (fruits, herbs and spices, etc.), herbal extracts, energy supplements, dietary supplements, pharmaceuticals, over the counter drugs, sleep aids, appetite suppressants, weight gain agents, antioxidants, nutraceuticals, confections, etc., and combinations thereof.

Useful characteristics of membranes described herein can provide sufficient barriers to environments and environmental changes, thereby protecting food compositions, probiotics and prebiotics, by, for example, prevention or attenuation of dehydration, providing oxygen barriers, thermal insulation, etc., that increase the length of time the probiotic and functional food as a whole remains viable.

### Encapsulated Compositions

Functional foods are desired to provide health and medical benefits, including the prevention and treatment of disease. Other factors also are considered to create a product that is commercially available (e.g. longer shelf life, product stability, etc.) and elicits a pleasurable experience for the consumer (e.g., flavor, ease of consumption, mouthfeel, etc.). Therefore, some considerations for the choice of food composition used as a carrier for the bioavailable compound include consumer taste preference, sufficiency to support desired concentrations of bioavailable compounds, compatibility of chemical environment between composition and bioavailable compound, compatibility of flavors of bioavailable compound and composition, and health and/or nutritional content of the composition.

Viability considerations for the choice of probiotic microorganism are influenced by the environment provided by the food composition in which the probiotic is delivered and ingested. Such considerations include, for example, a physiologically active state within the edible product, environmental temperature, pH, water activity, oxygen level, toxicity of the ingredients of the food product, potential for shear forces encountered during standard food production, and deleterious effects from freeze/thaws during storage and transport. Choice of probiotic can therefore influence choice of food composition, and vice versa, for the composition used as a delivery vehicle.

Taste to the consumer is important and can provide an enjoyable gustatory experience. Enjoyable food compositions and flavors can increase compliance of usage by consumers (e.g. children and elderly) and patients under clinical administration of the functional food. For example, typical flavorings for use in the compositions described herein include chocolate, vanilla, mints, natural fruit flavors, etc.

Edible and potable substances contemplated for the functional food include, but are not limited to, frozen dairy products such as frozen yogurts, ice creams, sorbets, gelatos, etc., other dairy products such as yogurt, fruits, vegetables, meat, a carbohydrate food product, botanicals, confections, and combinations thereof. The substances can be in natural form, puree form, frozen, soft, etc.

Other edible and potable substances that can be used as carriers of the bioavailable compounds include liquids such as water, alcoholic beverages and mixed drinks, juices, coffee and tea products, soft drinks, liquid confectionary beverages, and combinations thereof.

Additionally, various vitamins, minerals, supplements and nutraceuticals can fortify the composition, in addition to the bioavailable compounds used to make the functional food. In general, nutraceuticals provide a physiological benefit or provide protection against chronic disease. Such products may range from isolated nutrients, dietary supplements and specific diets to genetically engineered foods, herbal products, and processed foods such as cereals, soups, and beverages.

### Preparation of an Edible, Membrane Encased Composition

A membrane can be engineered for various functional food applications by being, for example, stronger, thinner/thicker, or taste a particular way, with methods in addition to adjusting the properties of an alginate solution. Adding suspended particles of food, nutraceuticals, compounds beneficial to gastrointestinal health, or other particles at least partially insoluble in water can achieve desirable properties important to the consumer and patient.

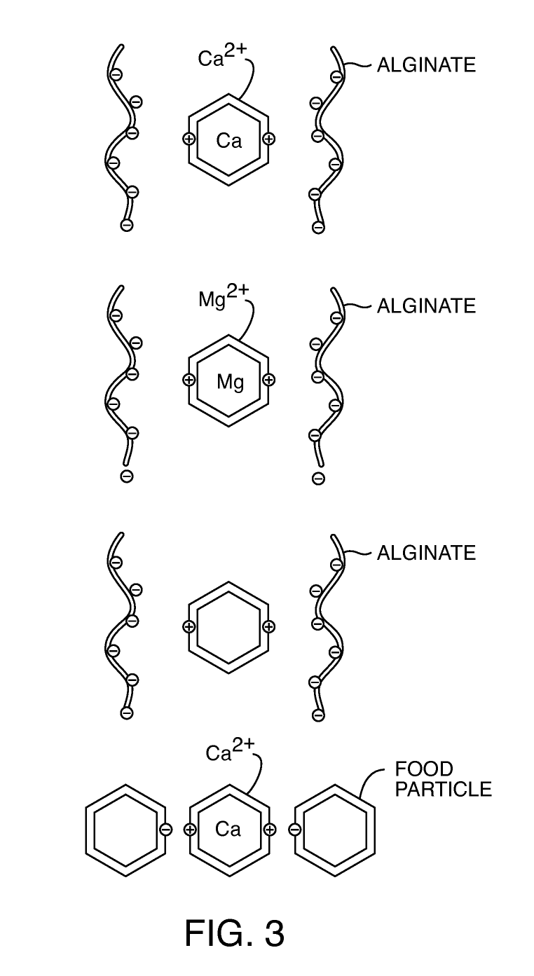

Often the added particles will be charged (i.e., most particle surfaces have some charge or zeta potential). This charge can be modified by the way each particle is created, its size, and the nature of the particle surface. Surfactants can be added to enhance the charged nature and the ionic atmosphere of the water can also be modified beneficially. When in solution (e.g., alginate or an aqueous medium), these particles can undergo strong or weak associations with alginate. When in contact with calcium, for example, particles will form with alginate a gelled membrane through interaction of the calcium and food particles trapped within the membrane, possibly strengthening it, improving flavor, etc. FIG. 3 schematically illustrates the interaction between positively charged particles (e.g., Ca²⁺ or Mg²⁺) with negatively charged alginate or food particles.

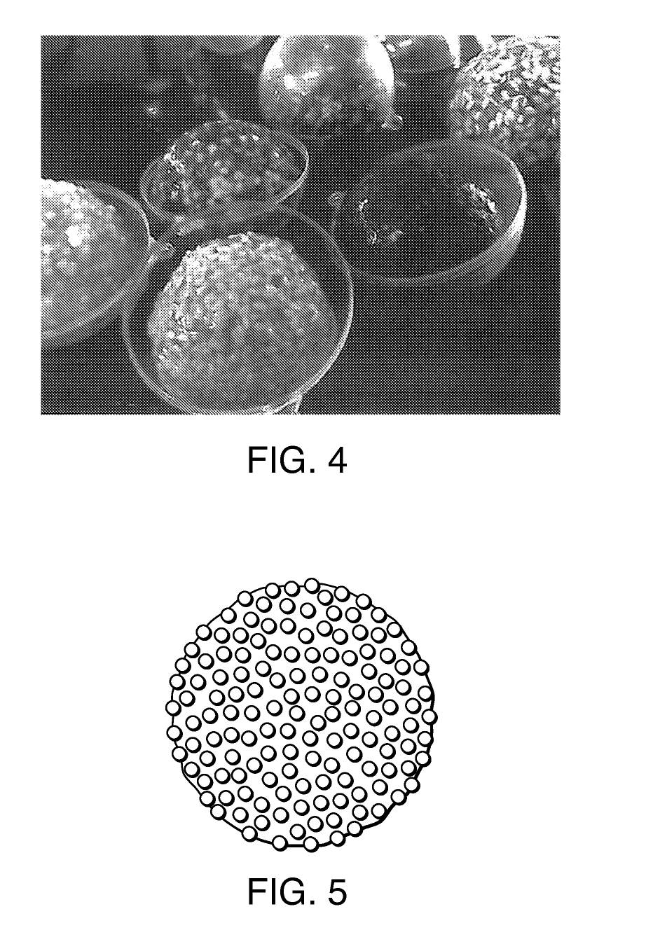

FIG. 4 illustrates various food compositions having membrane layers containing different particles (e.g., edible particles). By way of example, membrane layers can include differently sized particles, different types of particles, or different orientations or configurations of particles. The functional food compositions can be sized to various diameters, from greater than 1.5 centimeters to 2 centimeters, 3 centimeters, 4 centimeters, 5 centimeters, 7.5 centimeters, 10 centimeters, 15 centimeters, or 20 centimeters, or greater. Additionally, the functional food compositions can be enclosed in various shells for packaging, transportation, or storage.

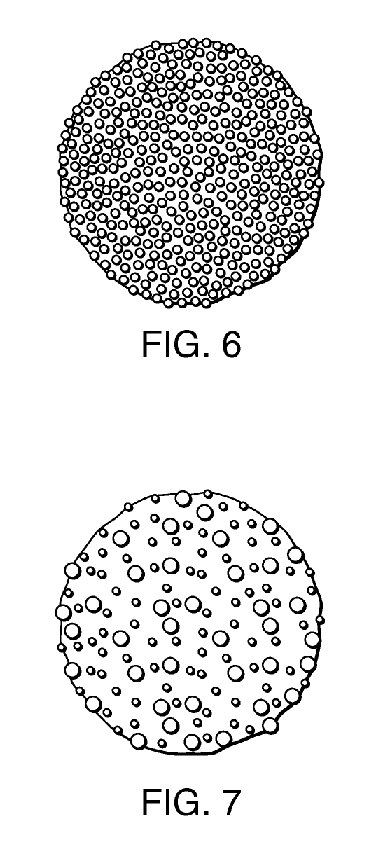

Referring to FIGS. 5, 6 and 7 a membrane layer around an ingestible substance can include larger and/or smaller particles suspended in the alginate polymer matrix, providing structural stability to the membrane and helping reduce deformation of the membrane. Such a membrane can have an unusual (e.g., non-spherical) shape. Additionally, particles can reduce evaporation of the aqueous portion of the membrane and/or the fluid inside the membrane, and provide a more rigid and/or less sticky surface for holding the composition.

The membrane layer having both large and small particles has been shown to produce better particle packing and arrangement within the membrane layer, possibly better structural integrity, reduced water evaporation from the membrane or the fluid contained therein, and forming more useful textures than membrane layers having only large or small particles.

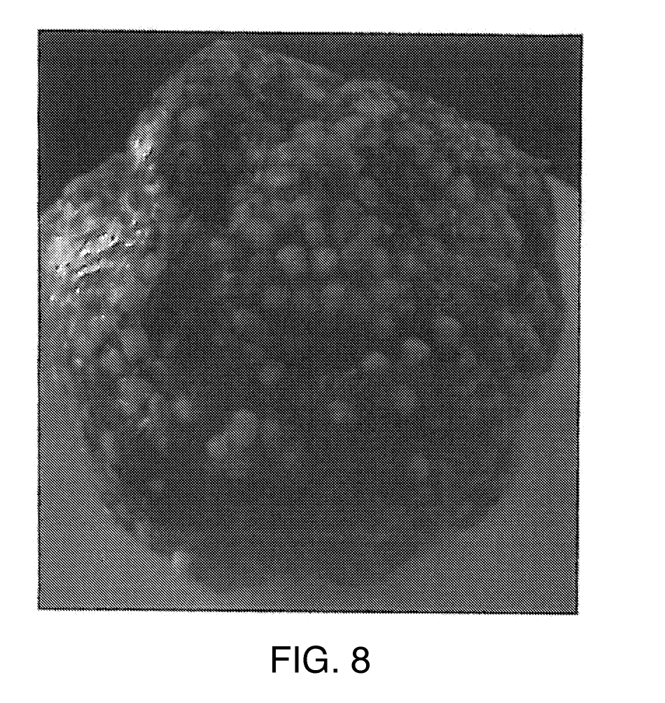

Functional food compositions can be formed as non-spherical, non-uniform shapes. Referring to FIG. 8, a membrane can include ridges or features for aesthetic and/or structural purposes. The functional food composition can be made to resemble naturally occurring objects (e.g., fruits and vegetables) to enhance the appearance of the product as a natural alternative to various pills shaped supplements and the like

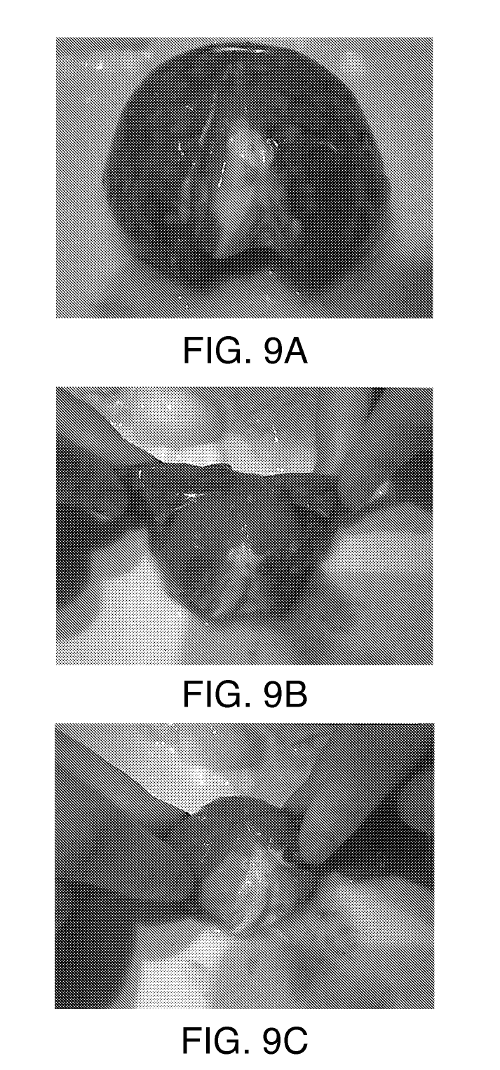

Referring to FIG. 9a-c, some functional food compositions can be made with multiple layers of membranes with particles embedded therein, and further have particulate layers between the membrane matrix layers, encasing an edible or potable substance. For example, a probiotic can be embedded in one membrane, and the prebiotic can be embedded in another membrane. In other alternatives, more than one probiotic or prebiotic can be used and isolated or embedded in separate membranes of the multi-membrane product.

### EXAMPLE 1

### Gastrointestinal Health Functional Food Composition

Membrane layer and inner compositions to be used in the functional nutrition transport systems can include various ingredients to achieve different products (e.g., different flavors, textures and ingredients addressing specific health purposes) based on the requirements or needs of the intended end user as well as recommended daily values or values sufficient to achieve a health benefit. Three activities prebiotic and probiotics are thought to participate in are changing the microbiota, fermentation producing organic acids, producing a health benefit to the host. Various health benefits include improved digestion, reduced intestinal gas, increased stool transit time, reduced diarrhea, improved immune function, increased probiotic CFU counts in stool, etc. An example of membrane layer composition and inner composition to be enclosed by the membrane are provided below in Tables 1a/b for a prebiotic/probiotic nutritional product for use in enhancing digestive health and/or immunity.

TABLE 1a
Inner Frozen Yogurt

| | Amount per individual edible | |
|---|---|---|
| Ingredient | transport vessel | |
| Frozen yogurt (e.g. vanilla) | 5-15 | g |
| Howaru Dophilus (Danisco) | 100 | BCFU |
| Howaru Bifidus (Danisco) | 100 | BCFU |
| BC30 (B. coagulans, Ganaden | 15 | BCFU |
| Biotech) | | |

TABLE 1b
Membrane

| | Amount per individual edible | |
|---|---|---|
| Ingredient | transport vessel | |
| Sodium Alginate | 0.01-0.4 | g |
| Fructo-Oligosaccharide, | 3 | g (up to about 30 grams, |
| Galacto-oligosaccharide, Inulin, | | optionally) |
| chicory root, etc. (eg. VitaFiber) | | |
| Water | 1.0-4.0 | |
| Calcium lactate | <0.5 | g |
| Fruit/Flavoring | 1.0-15 | g |
| Sweetener | 0.25-1.0 | g |
| Sodium Citrate (optional) | 0.025-0.1 | g |
| Stabilizer | 0.025-0.1 | g |

### EXAMPLE 2

### Integument Health Functional Food Composition

Membrane layer and inner compositions to be used in the functional food compositions can include various ingredients to achieve different products (e.g., different flavors, textures and ingredients addressing specific health purposes) based on the requirements or needs of the intended end user as well as recommended daily values or values sufficient to achieve a health benefit, as described herein. An example of membrane layer composition and inner composition to be enclosed by the membrane are provided below in Tables 2a/b for a bioflavonoid, carotene and elastic tissue component product, to deliver daily recommended or desired concentrations bioavailable compounds for beauty and health and wellness. Concentrations can be varied to provide the same concentrations in more than one serving.

TABLE 2a
Inner Ice Cream

| Ingredient | Amount per serving | |
|---|---|---|
| Non- or low-fat ice cream | 5-20 | g |
| (vanilla, chocolate) | | |
| Calcium lactate pentahydrate | 155 | mgs |
| Magnesium amino acid chelate | 155 | mgs |
| Zinc gluconate dihydrate | 7 | mgs |

TABLE 2b
Membrane

| Ingredient | Amount per serving | |
|---|---|---|
| Sodium Alginate | 0.01-0.4 | g |
| Water | 3.0-12.0 | g |
| Calcium lactate | <0.5 | g |
| Chocolate (cacao paste, | 1.0-4.0 | g |
| optionally bar chocolate) | | |
| Sweetener (granulated sugar, | 0.6-2.4 | g |
| trimoline, dehydrated glucose | | |
| syrup) | | |
| Cream | 0.3-1.2 | g |
| Sodium Citrate (optional) | 0.025-0.1 | g |
| Cocoa flavanol | 3-300 | mgs cocoa flavanol |
| | | containing about 75% |
| | | epicatechin and 25% catechin |
| Collagen | 1250 | mgs |
| Lutein | 25 | mgs |
| Silicic acid (Horsetail) | 70 | mgs |
| Biotin | 1.3 | mgs |
| Vitamin C | 10 | mgs |

### EXAMPLE 3

### Weight Loss Functional Food Composition

Membrane layer and inner compositions to be used in the functional food compositions can include various ingredients to achieve different products (e.g., different flavors, textures and ingredients addressing specific health purposes) based on the requirements or needs of the intended end user as well as recommended daily values or values sufficient to achieve a health benefit, as described herein. An example of membrane layer composition and inner composition to be enclosed by the membrane are provided below in Tables 3a/b for a weight loss product, to deliver daily recommended or desired concentrations for weight loss and treatment of weight related related disorders and diseases. Concentrations can be varied to provide the same concentrations in more than one serving.

TABLE 3a
Inner Ice Cream

| Ingredient | Amount per serving |
|---|---|
| Low fat, low cholesterol ice | 5-20 g |
| cream (e.g. mint) | |
| Appethyl | 0.1-5.0 g |

TABLE 3b
Membrane

| Ingredient | Amount per serving | |
|---|---|---|
| Sodium Alginate | 0.01-0.4 | g |
| Water | 3.0-12.0 | g |
| Calcium lactate | <0.5 | g |
| Chocolate (cacao paste, | 1.0-4.0 | g |
| optionally bar chocolate) | | |
| Sweetener (granulated sugar, | 0.6-2.4 | g |
| trimoline, dehydrated glucose | | |
| syrup) | | |
| Cream | 0.3-1.2 | g |
| Sodium Citrate (optional) | 0.025-0.1 | g |
| Stabilizer | 0.025-0.1 | g |
| Appethyl | 0.1-5.0 | g |

### EXAMPLE 4

### Skeletal Health Functional Food Composition

Membrane layer and inner compositions to be used in the functional food compositions can include various ingredients to achieve different products (e.g., different flavors, textures and ingredients addressing specific health purposes) based on the requirements or needs of the intended end user as well as recommended daily values or values sufficient to achieve a health benefit, as described herein. An example of membrane layer composition and inner composition to be enclosed by the membrane are provided below in Tables 4a/b for a skeletal health product, to deliver daily recommended or desired concentrations for bone maintenance. Concentrations can be varied to provide the same concentrations in more than one serving.

TABLE 4a
Inner Ice Cream

| | Amount per individual |
|---|---|
| Ingredient | serving |
| Ice cream (e.g. chocolate) | 5-20 g |

TABLE 4b
Membrane

| | Amount per individual | |
|---|---|---|
| Ingredient | serving | |
| Sodium Alginate | 0.01-0.4 | g |
| Calcium citrate malate | 200-1200 | mg |
| Magnesium citrate | 80-320 | mg |
| Vitamin D | 500-4000 | IU |
| Water | 3.0-12.0 | |
| Calcium lactate | <0.5 | g |
| Chocolate (cacao paste, | 1.0-4.0 | g |
| optionally bar chocolate) | | |
| Sweetener (granulated sugar, | 0.6-2.4 | g |
| trimoline, dehydrated glucose | | |
| syrup) | | |
| Cream | 0.3-1.2 | g |
| Sodium Citrate (optional) | 0.025-0.1 | g |
| Stabilizer | 0.025-0.1 | g |

### EXAMPLE 5

### Cardiovascular Health Functional Food Composition

Membrane layer and inner compositions to be used in the functional food compositions can include various ingredients to achieve different products (e.g., different flavors, textures and ingredients addressing specific health purposes) based on the requirements or needs of the intended end user as well as recommended daily values or values sufficient to achieve a health benefit, as described herein. An example of membrane layer composition and inner composition to be enclosed by the membrane are provided below in Tables 5a/b for a cardiovascular health composition to deliver daily recommended or desired concentrations for bioflavonoids, carotenes and essential fatty acid compounds. Concentrations can be varied to provide the same concentrations in more than one serving.

TABLE 5a
Inner Ice Cream

| Ingredient | Amount per serving |
|---|---|
| Non- or low-fat ice cream | 5-20 g |
| (vanilla, chocolate) | |
| Life's Microencapsulated DHA | 1000 mgs |

TABLE 5b
Membrane

| Ingredient | Amount per serving | |
|---|---|---|
| Sodium Alginate | 0.01-0.4 | g |
| Water | 3.0-12.0 | g |
| Calcium lactate | <0.5 | g |
| Chocolate (cacao paste, | 1.0-4.0 | g |
| optionally bar chocolate) | | |
| Sweetener (granulated sugar, | 0.6-2.4 | g |
| trimoline, dehydrated glucose | | |
| syrup) | | |
| Cream | 0.3-1.2 | g |
| Sodium Citrate (optional) | 0.025-0.1 | g |
| Stabilizer | 0.025-0.1 | g |
| Lycopene | 3 | milligrams |
| Cocoa flavanols | 250 | milligrams |

### EXAMPLE 6

### Joint and Cartilage Health Functional Food Composition

Membrane layer and inner compositions to be used in the functional food compositions can include various ingredients to achieve different products (e.g., different flavors, textures and ingredients addressing specific health purposes) based on the requirements or needs of the intended end user as well as recommended daily values or values sufficient to achieve a health benefit, as described herein. An example of membrane layer composition and inner composition to be enclosed by the membrane are provided below in Tables 6a/b for a joint and cartilage health product, to deliver daily recommended or desired concentrations for joint and cartilage maintenance and treatment of related disorders and diseases. Concentrations can be varied to provide the same concentrations in more than one serving.

TABLE 6a
Inner Ice Cream

| | Amount per individual |
|---|---|
| Ingredient | serving |
| Ice cream (e.g. chocolate) | 5-20 g |

TABLE 6b
Membrane

| Ingredient | Amount per serving | |
|---|---|---|
| Sodium Alginate | 0.01-0.4 | g |
| Water | 3.0-12.0 | |
| Calcium lactate | <0.5 | g |
| Chocolate (cacao paste, | 1.0-4.0 | g |
| optionally bar chocolate) | | |
| Sweetener (granulated sugar, | 0.6-2.4 | g |
| trimoline, dehydrated glucose | | |
| syrup) | | |
| Cream | 0.3-1.2 | g |
| Sodium Citrate (optional) | 0.025-0.1 | g |
| Stabilizer | 0.025-0.1 | g |
| Glucosamine | Up to 2000 | mg |
| Chondroitin | Up to 2000 | mg |

### EXAMPLE 7

### Multivitamin Functional Food Composition

Membrane layer and inner compositions to be used in the functional food compositions can include various ingredients to achieve different products (e.g., different flavors, textures and ingredients addressing specific health purposes) based on the requirements or needs of the intended end user as well as recommended daily values or values sufficient to achieve a health benefit, as described herein. An example of membrane layer composition and inner composition to be enclosed by the membrane are provided below in Tables 7a/b for a multivitamin product, to deliver daily recommended or desired concentrations for multivitamins and treatment for malnutrition and dietary imbalances. Concentrations can be varied to provide the same concentrations in more than one serving.

TABLE 7a
Inner Ice Cream

| Ingredient | Amount per serving |
|---|---|
| Chocolate ice cream | 5-20 g |

TABLE 7b
Membrane

| Ingredient | Amount per serving | |
|---|---|---|
| Sodium Alginate | 0.01-0.4 | g |
| Water | 3.0-12.0 | g |
| Calcium lactate | <0.5 | g |
| Chocolate (cacao paste, | 1.0-4.0 | g |
| optionally bar chocolate) | | |
| Sweetener (granulated sugar, | 0.6-2.4 | g |
| trimoline, dehydrated glucose | | |
| syrup) | | |
| Cream | 0.3-1.2 | g |
| Sodium Citrate (optional) | 0.025-0.1 | g |
| Stabilizer | 0.025-0.1 | g |
| AlternaVites ™ (for kids) | 2.5 | g |

AlternaVites for kids is a commercially available multivitamin supplement composition labeled to contain 50% RDA vitamin A, 100% RDA vitamin C, 125% RDA vitamin D, 100% RDA vitamin E, 100% RDA thiamin, 100% RDA riboflavin, 100% RDA niacin, 1005 RDA vitamin B6, 100% RDA folic acid, 100% RDA vitamin B12, 17% RDA biotin, 100% RDA pantothenic acid, 15% RDA calcium, 100% RDA iodine, 10% RDA magnesium, 100% RDA zinc, and 50% RDA manganese, with other sweeteners and flavorings.

### EXAMPLE 8

### Essential Fatty Acids Functional Food Composition

Membrane layer and inner compositions to be used in the functional food compositions can include various ingredients to achieve different products (e.g., different flavors, textures and ingredients addressing specific health purposes) based on the requirements or needs of the intended end user as well as recommended daily values or values sufficient to achieve a health benefit, as described herein. An example of membrane layer composition and inner composition to be enclosed by the membrane are provided below in Tables 8a/b for an essential fatty acid and dietary fiber product, to deliver daily recommended or desired concentrations for DHA for developmental health and dietary fiber for gastrointestinal health. Concentrations can be varied to provide the same concentrations in more than one serving.

TABLE 8a
Inner Ice Cream

| Ingredient | Amount per serving |
|---|---|
| Non-fat (vanilla) ice cream | 5-20 g |
| Life's Microencapsulated DHA | 500 mgs |

TABLE 8b
Membrane

| Ingredient | Amount per serving | |
|---|---|---|
| Sodium Alginate | 0.01-0.4 | g |
| Water | 3.0-12.0 | g |
| Calcium lactate | <0.5 | g |
| Chocolate (cacao paste, | 1.0-4.0 | g |
| optionally bar chocolate) | | |
| Sweetener (granulated sugar, | 0.6-2.4 | g |
| trimoline, dehydrated glucose | | |
| syrup) | | |
| Cream | 0.3-1.2 | g |
| Sodium Citrate (optional) | 0.025-0.1 | g |
| Stabilizer | 0.025-0.1 | g |
| Dietary fiber (e.g. inulin) | 3 | grams |

### EXAMPLE 9

### Branched-Chain Amino Acid Functional Food Composition

Membrane layer and inner compositions to be used in the functional food compositions can include various ingredients to achieve different products (e.g., different flavors, textures and ingredients addressing specific health purposes) based on the requirements or needs of the intended end user as well as recommended daily values or values sufficient to achieve a health benefit, as described herein. An example of membrane layer composition and inner composition to be enclosed by the membrane are provided below in Tables 9a/b for BCAA (and glutamine) functional food product, to deliver daily recommended or desired concentrations bioavailable compounds for general health and wellness. Concentrations can be varied to provide the same concentrations in more than one serving.

TABLE 9a
Inner ice cream

| Ingredient | Amount per serving |
|---|---|
| Ice cream | 5-20 grams |
| BCAA composition (Vitamin | 3.5 grams |
| Shoppe Body Tech BCAA + | |
| Glutamine product) | |

TABLE 9b
Membrane

| Ingredient | Amount per serving | |
|---|---|---|
| Sodium Alginate | 0.01-0.4 | g |
| Water | 3.0-12.0 | g |
| Calcium lactate | <0.5 | g |
| Sweetener (granulated sugar, | 0.6-2.4 | g |
| trimoline, dehydrated glucose | | |
| syrup) | | |
| Sodium Citrate (optional) | 0.025-0.1 | g |
| Stabilizer | 0.025-0.1 | g |
| Mineral supplement blend, | 10%-20% | RDA |
| powder form | | |

### EXAMPLE 10

### Antioxidant Functional Food Composition

Membrane layer and inner compositions to be used in the functional food compositions can include various ingredients to achieve different products (e.g., different flavors, textures and ingredients addressing specific health purposes) based on the requirements or needs of the intended end user as well as recommended daily values or values sufficient to achieve a health benefit, as described herein. An example of membrane layer composition and inner composition to be enclosed by the membrane are provided below in Tables 10a/b for an antioxidant functional food product, to deliver daily recommended or desired concentrations bioavailable compounds for general health and wellness. Concentrations can be varied to provide the same concentrations in more than one serving.

TABLE 10a
Inner frozen yogurt

| Ingredient | Amount per serving |
|---|---|
| Frozen yogurt | 5-20 grams |
| Fiber | 3-5 grams |

TABLE 10b
Membrane

| Ingredient | Amount per serving | |
|---|---|---|
| Sodium Alginate | 0.01-0.4 | g |
| Water | 3.0-12.0 | g |
| Calcium lactate | <0.5 | g |
| Sweetener (granulated sugar, | 0.6-2.4 | g |
| trimoline, dehydrated glucose | | |
| syrup) | | |
| Sodium Citrate (optional) | 0.025-0.1 | g |
| Stabilizer | 0.025-0.1 | g |
| Vita-Veggie ® High-ORAC fruit | 1-2 | grams |
| and veggie blend | | |
| Fresh fruit particles, chopped or | 2 | grams |
| minced | | |

### EXAMPLE 11

### Functional Food Composition

Membrane layer and inner compositions to be used in the functional nutrition transport systems can include various ingredients to achieve different products (e.g., different flavors, textures and ingredients addressing specific health purposes) based on the requirements or needs of the intended end user as well as recommended daily values or values sufficient to achieve a health benefit. Various health benefits include improved immune response, improved vaccination response, modulated hypersensitive immune response, cancer treatment, fungal infection treatment, etc. An example of membrane layer composition and inner composition to be enclosed by the membrane are provided below in Tables 11a/b for a β-glucan/probiotic nutritional product for use in enhancing immunity.

TABLE 11a
Inner Frozen Yogurt

| | Amount per individual |
|---|---|
| Ingredient | serving |
| Frozen yogurt (e.g. vanilla) | 5-20 g |
| Live probiotic: | 10 BCFU, total (1-100 BCFU, |
| L. acidophilus (Danisco | optionally) |
| HowAreU); Bifidus (Danisco) | |
| Heat treated probiotic: | 10 mg |
| Lactobacillus plantarum (House | |
| Wellness Immuno20 HL L137) | |

TABLE 11b
Membrane

| | Amount per individual | |
|---|---|---|
| Ingredient | serving | |
| Sodium Alginate | 0.01-0.4 | g |
| β-glucan (Wellmune Biothera) | 250 | mg |
| Water | 1.0-4.0 | |
| Calcium lactate | <0.5 | g |
| Fruit/flavoring | 1.0-15 | g |
| Sweetener | 0.25-1.0 | g |
| Sodium citrate (optional) | 0.025-0.1 | g |
| Stabilizer | 0.025-0.1 | g |

In general, an edible transport vessel can be made as follows. In a pot, combine 15 g sodium alginate and into 985 g of mineral water, then heat over a low heat until it simmers. Mix until alginate is completely dissolved and solution has a uniform consistency. Let set at 4° C. for 2-3 hours. Add sugar, stabilizer, flavorings, and other membrane components to a final concentration desired for the individual transport vessels, and mix to a uniform consistency. Prepare a 2% calcium bath by mixing 20 g of calcium lactate with 1 liter water. Dissolve completely. Blend yogurt and sugar to consistent texture, and add to a pastry bag, piping bag or similar device. Dip end of pastry bag into inner membrane alginate solution, and form small spheres of 1-2 inch diameter. Alternatively, frozen, preformed spheres of an encapsulated food composition (e.g., frozen yogurt or ice cream with probiotic) can be placed into the alginate solution, with or without a first calcium bath dip. Remove spheres from membrane alginate solution and place into calcium bath for 10-15 minutes. Remove spheres and dry the surface with absorbing paper. Store at 4° C. or −20° C.

While certain combinations of membranes and inner compositions have been provided and described as being used together, other combinations are possible. It will be understood that various modifications may be made without departing from the spirit and scope of the invention.

1. A functional food composition, comprising:
an edible or potable substance;
a cross-linked edible matrix encapsulating the edible or potable substance;
a probiotic;
and a prebiotic, with the proviso that the edible or potable substance and the matrix do not both contain the probiotic or both contain the prebiotic.

2. The functional food composition of claim 1, wherein the matrix comprises the prebiotic and the edible or potable substance component comprises the probiotic.

3. The functional food composition of claim 1, wherein the matrix comprises the probiotic and the edible or potable substance comprises the prebiotic.

4. The functional food composition of claim 1, wherein the composition comprises a probiotic concentration from about 1.0 to about 100 billion colony forming units in an individual edible transport vessel.

5. The functional food composition of claim 1, wherein the probiotic is an organism selected from the genera group consisting of Aspergillus, Bacillus, Bacteroides, Bifidobacterium, Brettanomyces, Enterococcum, Kluyveromyces, Lactobacillus, Lactococcus, Leuconostoc, Pediococcus, Propionibacterium, Saccharomyces, Shewanella, Streptococcus, Torulaspora, Vagococcus, and any derivatives, strains, and combinations thereof.

6. The functional food composition of claim 1, wherein the prebiotic is one of the group consisting of galacto-oligosaccharides, inulin, oligofructose, isomalto-oligosaccharides, lactulose, lactosucrose, transgalacto-oligosaccharides, soybean oligosaccharides, tagatose, xylo-oligosaccharides, and combinations thereof.

7. The functional food composition of claim 1, wherein the composition comprises a prebiotic concentration from about 1.0 to about 30.0 grams in an individual edible transport vessel.

8. The functional food composition of claim 1, wherein the matrix is comprised of an edible polymer charge cross-linked by multivalent ions, including cross-linking interactions between the edible particles and edible polymer or plurality of edible polymers via bridges formed by the multivalent ions.

9. The functional food composition of claim 1, wherein the matrix comprises a polysaccharide selected from the group consisting of a hydrocolloid, shellac, and fibers.

10. The functional food composition of claim 1, further comprising additional edible particles in the cross-linked matrix.

11-17. (canceled)

18. A method of preparing a functional food composition, comprising the steps of:
a) providing an edible or potable substance, an edible polymer, a probiotic, and a prebiotic;
b) combining the edible or potable substance with the probiotic or the prebiotic;
c) encapsulating the edible substance with the probiotic or the prebiotic in an edible matrix that comprises the edible polymer and the probiotic or the prebiotic; with the proviso that the edible or potable substance and the matrix do not both contain the probiotic or both contain the prebiotic.

19. The method of claim 18, further comprising cross-linking the edible matrix.

20. The method of claim 18, wherein the prebiotic is in the matrix and the probiotic is in the edible or potable substance.

21. The method of claim 18, wherein the probiotic is in the matrix and the prebiotic is in the edible or potable substance.

22. The method of claim 18, wherein the composition comprises a probiotic concentration from about 1.0 to about 100 billion colony forming units in an individual edible transport vessel.

23. The method of claim 18, wherein the probiotic is an organism selected from the genera group consisting of Aspergillus, Bacillus, Bacteroides, Bifidobacterium, Brettanomyces, Enterococcum, Kluyveromyces, Lactobacillus, Lactococcus, Leuconostoc, Pediococcus, Propionibacterium, Saccharomyces, Shewanella, Streptococcus, Torulaspora, Vagococcus, and any derivatives, strains, and combinations thereof.

24. The method of claim 18, wherein the prebiotic is one of the group consisting of galacto-oligosaccharides, inulin, oligofructose, isomalto-oligosaccharides, lactulose, lactosucrose, transgalacto-oligosaccharides, soybean oligosaccharides, tagatose, xylo-oligosaccharides, and combinations thereof.

25-520. (canceled)

521. The functional food composition of claim 1, wherein the probiotic is a spore forming organism.

522. The functional food composition of claim 1, wherein the probiotic is an organism comprising Bacillus coagulans, and any derivatives, strains, and combinations thereof.

523. The method of claim 18, wherein the probiotic is an organism comprising Bacillus coagulans, and any derivatives, strains, and combinations thereof.
